(19) United States
(12) Patent Application Publication
(10) Pub. No.: US 2025/0326847 A1
(43) Pub. Date: Oct. 23, 2025

(54) ACTIVATABLE ANTI-CTLA4 ANTIBODIES FOR TREATING CANCER

(71) Applicant: Adagene Pte. Ltd., Singapore (SG)

(72) Inventors: Peter Peizhi LUO, Morgan Hill, CA (US); Songmao ZHENG, West Harrison, IN (US); JiPing ZHA, San Diego, CA (US); Guizhong LIU, Fort Lee, NJ (US); Xiaohong SHE, Suzhou, Jiangsu (CN)

(73) Assignee: Adagene Pte. Ltd., Singapore (SG)

(21) Appl. No.: 19/110,046

(22) PCT Filed: Sep. 8, 2023
(86) PCT No.: PCT/US2023/073787

§ 371 Date: Mar. 7, 2025

Related U.S. Application Data
(60) Provisional application No. 63/405,293, filed on Sep. 9, 2022.
(60) Provisional application No. 63/495,965, filed on Apr. 13, 2023.

(51) Int. Cl. C07K 16/28; A61K 39/00; A61P 35/00

(52) U.S. Cl. CPC C07K 16/2818; A61P 35/00; A61K 2039/507; A61K 2039/545; C07K 2317/565; C07K 2319/50; C07K 2319/70

(57) ABSTRACT

The present application provides compositions and methods for treating cancers using anti-cytotoxic T-lymphocyte-associated protein 4 (CTLA4) antibodies, such as activatable anti-CTLA4 antibodies. In some embodiments, combination therapies including an anti-CTLA4 antibody and a PD-1 inhibitor.

### CROSS-REFERENCE TO RELATED APPLICATIONS

This application claims priority to U.S. Provisional Patent Application No. 63/405,293, filed Sep. 9, 2022, and U.S. Provisional Patent Application No. 63/495,965, filed Apr. 13, 2023, the disclosures which are hereby incorporated herein by reference in their entireties for all purposes.

### FIELD OF THE INVENTION

The present application is in the field of cancer therapeutics, and relates to antibodies that bind to human Cytotoxic T-lymphocyte Protein 4 (CTLA4).

### SUBMISSION OF SEQUENCE LISTING ON ASCII TEXT FILE

The contents of the electronic sequence listing (6954020024xxSEQLIST.xml; Size: 364,433 bytes; and Date of Creation: Sep. 5, 2023) is herein incorporated by reference in its entirety.

### BACKGROUND

Cytotoxic T-lymphocyte Protein 4 (CTLA4) is a member of the immunoglobulin (Ig) superfamily of proteins that acts to downregulate T-cell activation and maintain immunogenic homeostasis. It has been shown that in vivo antibody-mediated blockade of CTLA4 enhanced anti-cancer immune responses in a syngeneic murine prostate cancer model (Kwon et al. (1997) Proc Natl Acad Sci USA, 94(15):8099-103). In addition, blockade of CTLA4 function was shown to enhance anti-tumor T cell responses at various stages of tumor growth in tumor-bearing mice (Yang et al. (1997) Cancer Res 57(18):4036-41; Hurwitz et al. (1998) Proc Natl Acad Sci USA 95 (17):10067-7). However, the development of antibody-based therapeutics suitable for human use remains difficult, as translation from pre-clinical animal models to human safety is often poor. Accordingly, a need exists for anti-CTLA4 antibodies that are cross-reactive among different species, such as humans and experimental animals (e.g., mouse, monkey, rat, etc.), to concurrently enable animal model studies and provide suitable human therapeutic candidates. In addition, a need exists for the development of safer anti-CTLA4 antibodies that are only active in certain contexts, such as in the protease-rich tumor microenvironment.

### BRIEF SUMMARY

The present disclosure provides methods of treating cancer with anti-CTLA4 antibodies and activatable anti-CTLA4 antibodies. The present application further provides methods for treating cancer with an anti-CTLA4 antibody and a second therapeutic agent. The present application further provides methods for treating cancer with an activatable anti-CTLA4 antibody and a second therapeutic agent. The present application further provides methods for treating cancer with an anti-CTLA4 antibody and at least two additional (e.g. two or three) therapeutic agents.

In one aspect, provided herein is a method of treating a cancer in a subject, comprising administering to the subject: (a) an effective amount of an activatable antibody, wherein the activatable antibody comprises: a polypeptide comprising, from N-terminus to C-terminus, a masking moiety (MM), a cleavable moiety (CM), and a target binding moiety (TBM), wherein the MM comprises an amino acid sequence selected from the group consisting of XₘCPDHPYPCXX (SEQ ID NO:181), XₘCDAFYPYCXX (SEQ ID NO:182), XₘCDSHYPYCXX (SEQ ID NO:183), and XₘCVPYYYACXX (SEQ ID NO:184), and where m is from 2-10, and where each X is independently an amino acid selected from the group consisting of A, C, D, E, F, G, H, I, K, L, M, N, P, Q, R, S, T, V, W, and Y; wherein the MM inhibits the binding of the activatable antibody to human CTLA4 when the CM is not cleaved; wherein the CM comprises at least a first cleavage site; wherein: a) the TBM comprises an antibody light chain variable region (VL), and the activatable antibody further comprises a second polypeptide comprising an antibody heavy chain variable region (VH); b) the TBM comprises an antibody heavy chain variable region (VH), and the activatable antibody further comprises a second polypeptide comprising an antibody light chain variable region (VL); c) the TBM comprises from the N-terminus to the C-terminus, an antibody light chain variable region (VL) and an antibody heavy chain variable region (VH); or d) the TBM comprises from the N-terminus to the C-terminus, an antibody heavy chain variable region (VH) and an antibody light chain variable region (VL); wherein the activatable antibody binds to human CTLA4 via the VH and VL when the CM is cleaved. In some embodiments, the subject is human. In some embodiments, the subject is a non-human animal or non-human mammal.

In some embodiments, the activatable antibody comprises: a polypeptide comprising, from N-terminus to C-terminus, a masking moiety (MM), a cleavable moiety (CM), and an anti-CTLA4 antibody as described herein, wherein the MM comprises an amino acid sequence EVGSYPNPSSDCVPYYYACAY (SEQ ID NO:192), and the cleavable moiety comprises an amino acid sequence SGRSAGGGGTPLGLAGSGGS (SEQ ID NO:221). The MM and CM, from N-terminus to C-terminus comprises an amino acid sequence EVGSYPNPSSDCVPYYYACAY SGRSAGGGGTPLGLAGSGGS (SEQ ID NO:200). In particular embodiments, the MM and the CM are covalently attached to the N-terminus of the light chain of the anti-CTLA4 antibody. In some embodiments, the MM and CM, from N-terminus to C-terminus comprises an amino acid sequence that has at least 90% or at least 95% sequence identity to SEQ ID NO:200.

In some embodiments, the activatable anti-CTLA4 antibody can be administered as a monotherapy to a patient in need thereof. In other embodiments, the activatable anti-CTLA4 antibody can be administered in combination with one or more additional agents, as set forth herein. In some embodiments, the cancer is ovarian cancer, pancreatic cancer, cholangiocarcinoma, lung cancer, breast cancer, hepatocellular carcinoma, glioblastoma, renal cell carcinoma, head and neck squamous cell carcinoma, colorectal cancer, gastrointestinal stroma tumor (GIST) or endometrial carcinoma. In some embodiments, the lung cancer is non-small cell lung cancer (NSCLC). In some embodiments, the colorectal cancer is Microsatellite stable (MSS), microsatellite instability high (MSI-H) or mismatch repair deficient (dMMR+) colorectal cancer. In some embodiments, the melanoma is uveal (UV) melanoma. In some embodiments, the cancer is squamous cell carcinoma (SCC) (e.g., anal, anorectal, penile, or cutaneous). In some embodiments, the cancer is anal squamous cell carcinoma or penile squamous cell carcinoma.

In some embodiments, the MM of the activatable anti-CTLA4 antibody further comprises, at its N-terminus, an additional amino acid sequence. In some embodiments, the additional amino acid sequence comprises the amino acid sequence of SEQ ID NO: 148.

In some embodiments, the first cleavage site of the activatable anti-CTLA4 antibody is a protease cleavage site for a protease selected from the group consisting of urokinase-type plasminogen activator (uPA), matrix metalloproteinase-1 (MMP-1), MMP-2, MMP-3, MMP-8, MMP-9, MMP-14, Tobacco Etch Virus (TEV) protease, plasmin, Thrombin, Factor X, PSA, PSMA, Cathepsin D, Cathepsin K, Cathepsin S, ADAM10, ADAM12, ADAMTS, Caspase-1, Caspase-2, Caspase-3, Caspase-4, Caspase-5, Caspase-6, Caspase-7, Caspase-8, Caspase-9, Caspase-10, Caspase-11, Caspase-12, Caspase-13, Caspase-14, and TACE.

In some embodiments, the CM further comprises a first linker (L₁) C-terminal to the first cleavage site. In some embodiments, the L₁ comprises an amino acid sequence selected from the group consisting of SEQ ID NOS: 156-163.

In some embodiments, the CM further comprises a second cleavage site. In some embodiments, the second cleavage site is C-terminal to the L₁. In some embodiments, the second cleavage site is a protease cleavage site for a protease selected from the group consisting of urokinase-type plasminogen activator (uPA), matrix metalloproteinase-1 (MMP-1), MMP-2, MMP-3, MMP-8, MMP-9, MMP-14, Tobacco Etch Virus (TEV) protease, plasmin, Thrombin, Factor X, PSA, PSMA, Cathepsin D, Cathepsin K, Cathepsin S, ADAM10, ADAM12, ADAMTS, Caspase-1, Caspase-2, Caspase-3, Caspase-4, Caspase-5, Caspase-6, Caspase-7, Caspase-8, Caspase-9, Caspase-10, Caspase-11, Caspase-12, Caspase-13, Caspase-14, and TACE. In some embodiments, the first and second cleavage sites are different.

In some embodiments, the CM further comprises a second linker (L₂) C-terminal to the second cleavage site. In some embodiments, the L₂ comprises an amino acid sequence selected from the group consisting of SEQ ID NOS: 156-163. In some embodiments, the CM further comprises a third linker (L₃) N-terminal to the first cleavage site.

In some embodiments, the CM comprises at least a first protease cleavage site and is cleaved with one or more proteases selected from the group consisting of urokinase-type plasminogen activator (uPA), matrix metalloproteinase-1 (MMP-1), MMP-2, MMP-3, MMP-8, MMP-9, MMP-14, Tobacco Etch Virus (TEV) protease, plasmin, Thrombin, Factor X, PSA, PSMA, Cathepsin D, Cathepsin K, Cathepsin S, ADAM10, ADAM12, ADAMTS, Caspase-1, Caspase-2, Caspase-3, Caspase-4, Caspase-5, Caspase-6, Caspase-7, Caspase-8, Caspase-9, Caspase-10, Caspase-11, Caspase-12, Caspase-13, Caspase-14, and TACE.

In some embodiments, the activatable anti-CTLA4 antibody comprises an amino acid sequence selected from the group consisting of SEQ ID NOS: 165-179.

In some embodiments, the activatable anti-CTLA4 antibody has a MM amino acid sequence selected from the group consisting of SEQ ID NOS: 189-196. In other embodiments, the activatable anti-CTLA4 antibody comprises a MM amino acid sequence selected from the group consisting of SEQ ID NOS: 213-216. In other embodiments, the activatable anti-CTLA4 antibody comprises a MM amino acid sequence selected from the group consisting of SEQ ID NOS: 141-147. In particular embodiments, the activatable anti-CTLA4 antibody comprises a MM amino acid sequence of SEQ ID NO: 200.

In some embodiments, the activatable anti-CTLA4 antibody has a combined MM/CM amino acid sequence selected from the group consisting of SEQ ID NOS: 197-209. In particular embodiments, the activatable anti-CTLA4 antibody has a combined MM/CM amino acid sequence of SEQ ID NO: 192.

In some embodiments, upon cleavage of the MM, the activatable anti-CTLA4 antibody specifically binds to an epitope comprising amino acid residues Y105 and L106 of human CTLA4 but does not comprise residue 1108, wherein the numbering of the amino acid residues is according to SEQ ID NO: 207.

In some embodiments, upon cleavage of the MM, a) the cleaved anti-CTLA4 antibody binds to human CTLA4, cynomolgus monkey CTLA4, mouse CTLA4, rat CTLA4, and dog CTLA4 with a dissociation constant (K_{D}) of about 350 nM or less; b) binding of the anti-CTLA4 antibody induces antibody-dependent cell cytotoxicity (ADCC) against a CTLA4-expressing human cell or a human Treg cell, wherein the ADCC activity of the anti-CTL4 antibody is higher than the ADCC activity of ipilimumab; and/or c) the anti-CTLA4 antibody has an IC50 higher than the IC50 of ipilimumab for blocking binding of CD80 and/or CD86 to human CTLA4 in an assay wherein either when CD80 and/or CD86 are plate bound or when human CTLA4 is present on cell surface.

In some embodiments, the activatable anti-CTLA4 antibody comprises an HVR-H1 comprising the amino acid sequence of SEQ ID NO: 23, an HVR-H2 comprising the amino acid sequence of SEQ ID NO: 35, an HVR-H3 comprising the amino acid sequence of SEQ ID NO: 45, an HVR-L1 comprising the amino acid sequence of SEQ ID NO: 58, an HVR-L2 comprising the amino acid sequence of SEQ ID NO: 66, and an HVR-L3 comprising the amino acid sequence of SEQ ID NO: 75.

In some such embodiments, the activatable anti-CTLA4 antibody, upon cleavage, comprises: a) a heavy chain variable region comprising the amino acid sequence of SEQ ID NO: 87, and a light chain variable region comprising the amino acid sequence of SEQ ID NO: 100. In some of the foregoing embodiments, the anti-CTLA4 antibody comprises a heavy chain variable region comprising an amino acid sequence having at least 90% (e.g., at least 92%, at least 93%, at least 94%, at least 95%, at least 96%, at least 97%, at least 98% or at least 99%) sequence identity to the amino acid sequence of SEQ ID NO: 87, and/or a light chain variable region comprising the amino acid sequence having at least 90% (e.g., at least 92%, at least 93%, at least 94%, at least 95%, at least 96%, at least 97%, at least 98% or at least 99%) sequence identity to the amino acid sequence of SEQ ID NO: 100.

In some embodiments, the activatable anti-CTLA4 antibody comprises a heavy chain comprising the amino acid sequence of SEQ ID NO:320 and a light chain comprising the amino acid sequence of SEQ ID NO:322. The activatable antibody having heavy chain SEQ ID No: 320 and light chain SEQ ID No. 322 is referred to as TY21580. In some embodiments, the activatable antibody comprises a heavy chain comprising the amino acid sequence of SEQ ID NO:320 and a light chain comprising the amino acid sequence of SEQ ID NO:322. In some embodiments, the activatable anti-CTLA4 antibody comprises a heavy chain having at least 90% (e.g., at least 92%, at least 93%, at least 94%, at least 95%, at least 96%, at least 97%, at least 98% or at least 99%) sequence identity to the amino acid sequence of SEQ ID NO:320. In some embodiments, the activatable anti-CTLA4 antibody comprises a light chain having at least 90% (e.g., at least 92%, at least 93%, at least 94%, at least 95%, at least 96%, at least 97%, at least 98% or at least 99%) sequence identity to the amino acid sequence of SEQ ID NO:321. In some embodiments, the activatable anti-CTLA4 antibody comprises a heavy chain having at least 90% (e.g., at least 92%, at least 93%, at least 94%, at least 95%, at least 96%, at least 97%, at least 98% or at least 99%) sequence identity to the amino acid sequence of SEQ ID NO:320. In some embodiments, the activatable anti-CTLA4 antibody comprises a light chain having at least 90% (e.g., at least 92%, at least 93%, at least 94%, at least 95%, at least 96%, at least 97%, at least 98% or at least 99%) sequence identity to the amino acid sequence of SEQ ID NO:322. In some embodiments, the activatable antibody is TY22404.

In another aspect, the activatable anti-CTLA4 antibodies of the disclosure are administered to a subject in combination with a PD-1 inhibitor (e.g., an anti-PD-1 antibody). In other such embodiments, the anti-PD1 antibody is toripalimab. In some embodiments, the subject is human. In some embodiments, the subject is a non-human animal or non-human mammal. In some embodiments, the combination of the activatable anti-CTLA4 antibody and PD-1 inhibitor (e.g. anti-PD-1 antibody) show a synergistic effect when administered in combination. In some of the foregoing embodiments, the anti-CTLA4 antibody comprises a heavy chain variable region comprising the amino acid sequence of SEQ ID NO: 87 or an amino acid sequence having at least 90% (e.g., at least 92%, at least 93%, at least 94%, at least 95%, at least 96%, at least 97%, at least 98% or at least 99%) sequence identity to the amino acid sequence of SEQ ID NO: 87, and/or a light chain variable region comprising the amino acid sequence of SEQ ID NO: 100 or an amino acid sequence having at least 90% (e.g., at least 92%, at least 93%, at least 94%, at least 95%, at least 96%, at least 97%, at least 98% or at least 99%) sequence identity to the amino acid sequence of SEQ ID NO: 100. In some of the foregoing embodiments, the anti-CTLA4 antibody is an activatable anti-CTLA4 antibody that upon cleavage, comprises a heavy chain variable region comprising the amino acid sequence of SEQ ID NO: 87 or an amino acid sequence having at least 90% (e.g., at least 92%, at least 93%, at least 94%, at least 95%, at least 96%, at least 97%, at least 98% or at least 99%) sequence identity to the amino acid sequence of SEQ ID NO: 87, and/or a light chain variable region comprising the amino acid sequence of SEQ ID NO: 100 or an amino acid sequence having at least 90% (e.g., at least 92%, at least 93%, at least 94%, at least 95%, at least 96%, at least 97%, at least 98% or at least 99%) sequence identity to the amino acid sequence of SEQ ID NO: 100.

In some embodiments, provided herein is a method of treating a cancer in a subject, comprising administering to the subject: (a) an effective amount of an activatable antibody, wherein the activatable antibody comprises: a polypeptide comprising, from N-terminus to C-terminus, a masking moiety (MM), a cleavable moiety (CM), and an anti-CTLA4 antibody as described herein, wherein the MM comprises an amino acid sequence EVGSYPNPSSDCVPYYYACAY (SEQ ID NO:192), and the cleavable moiety comprises an amino acid sequence SGRSAGGGGTPLGLAGSGGS (SEQ ID NO:221); and (b) an effective amount of toripalimab. The MM and CM, from N-terminus to C-terminus comprises an amino acid sequence EVGSYPNPSSDCVPYYYACAY SGRSAGGGGTPLGLAGSGGS (SEQ ID NO:200). In particular embodiments, the MM and the CM are covalently attached to the N-terminus of the light chain of the anti-CTLA4 antibody. In some embodiments, the MM and CM, from N-terminus to C-terminus comprises an amino acid sequence that has at least 90% or at least 95% sequence identity to SEQ ID NO:200.

In some embodiments according to any one of the methods described above, the cancer is resistant or refractory to a prior therapy, wherein the prior therapy is an inhibitor of CTLA4, PD-1, or a PD-1 ligand. In some embodiments, the subject is resistant to or has relapsed from a prior therapy, wherein the prior therapy is an inhibitor of CTLA4, PD-1, or a PD-1 ligand. In some embodiments, the prior therapy is an inhibitor of CTLA4, such as an anti-CTLA4 antibody, for example ipilimumab. In some embodiments, the prior therapy is an inhibitor of PD-1, such as an anti-PD-1 antibody, for example, nivolumab or toripalimab. In some embodiments, the prior therapy is an inhibitor of a PD-1 ligand (e.g., PD-L1), for example an anti-PD-L1 antibody. In some embodiments, the prior therapy includes both an inhibitor of CTLA4 and am inhibitor of PD-1. In some embodiments, the prior therapy includes both an inhibitor of CTLA4 and an inhibitor of PD-L1. In some of the foregoing embodiments, the activatable anti-CTLA4 antibody, upon cleavage of the MM, comprises a heavy chain variable region comprising the amino acid sequence of SEQ ID NO: 87 or an amino acid sequence having at least 90% (e.g., at least 92%, at least 93%, at least 94%, at least 95%, at least 96%, at least 97%, at least 98% or at least 99%) sequence identity to the amino acid sequence of SEQ ID NO: 87, and/or a light chain variable region comprising the amino acid sequence of SEQ ID NO: 100 or an amino acid sequence having at least 90% (e.g., at least 92%, at least 93%, at least 94%, at least 95%, at least 96%, at least 97%, at least 98% or at least 99%) sequence identity to the amino acid sequence of SEQ ID NO: 100. In some of the foregoing embodiments, the activatable anti-CTLA4 antibody comprises a human IgG1 Fc region, such as a wild type IgG1 Fc region or a variant that has enhanced ADCC activity. In some of the foregoing embodiments, the activatable anti-CTLA4 antibody comprises a human IgG1 Fc region, such as a wild type IgG1 Fc region or a variant that has enhanced ADCC activity. In some of the foregoing embodiments, the activatable antibody comprises a heavy chain comprising the amino acid sequence of SEQ ID NO:320 or 321, or an amino acid sequence having at least 90% (e.g., at least 92%, at least 93%, at least 94%, at least 95%, at least 96%, at least 97%, at least 98% or at least 99%) sequence identity to the amino acid sequence of SEQ ID NO: 320 or 321, and a light chain comprising the amino acid sequence of SEQ ID NO:322, or an amino acid sequence having at least 90% (e.g., at least 92%, at least 93%, at least 94%, at least 95%, at least 96%, at least 97%, at least 98% or at least 99%) sequence identity to the amino acid sequence of SEQ ID NO: 322.

In any of the foregoing embodiments, the activatable anti-CTLA4 antibody (e.g., TY22404), when administered as a monotherapy or in combination with another agent, can be administered at a dose that provides a steady state concentration of the cleaved antibody (i.e., active antibody following cleavage of the masking moiety (MM) and cleavable moiety (CM)) above the EC50 value of the cleaved antibody. In some embodiments, the activatable anti-CTLA4 antibody (e.g., TY22404), when administered as a monotherapy or in combination with another agent, can be administered at a dose that provides a steady state concentration of the cleaved antibody above the EC50 value of the cleaved antibody. In some of the foregoing embodiments, the concentration of the cleaved antibody is measured at the trough level of the anti-CTLA4 antibody.

In any of the foregoing embodiments, the activatable anti-CTLA4 antibody (e.g., TY22404), when administered as a monotherapy or in combination with another agent, can be administered at a dose that provides a steady state concentration of the cleaved antibody (i.e., active antibody following cleavage of the masking moiety (MM) and cleavable moiety (CM)) of from about 100 nM to about 600 nM. In some embodiments, the activatable anti-CTLA4 antibody is administered at a dose that provides a steady state concentration of the cleaved antibody of from about 100 nM to about 175 nM. In some embodiments, the activatable anti-CTLA4 antibody is administered at a dose that provides a steady state concentration of the cleaved antibody of from about 100 nM to about 200 nM. In some embodiments, the activatable anti-CTLA4 antibody is administered at a dose that provides a steady state concentration of the cleaved antibody of from about 125 nM to about 200 nM. In some embodiments, the activatable anti-CTLA4 antibody is administered at a dose that provides a steady state concentration of the cleaved antibody of from about 100 nM to about 150 nM. In some embodiments, the activatable anti-CTLA4 antibody is administered at a dose that provides a steady state concentration of the cleaved antibody of from about 125 nM to about 175 nM. In some embodiments, the activatable anti-CTLA4 antibody is administered at a dose that provides a steady state concentration of the cleaved antibody of from about 125 nM to about 150 nM. In some embodiments, the activatable anti-CTLA4 antibody is administered at a dose that provides a steady state concentration of the cleaved antibody of from about 150 nM to about 200 nM. In some embodiments, the activatable anti-CTLA4 antibody is administered at a dose that provides a steady state concentration of the cleaved antibody of from about 200 nM to about 600 nM. In some embodiments, the activatable anti-CTLA4 antibody is administered at a dose that provides a steady state concentration of the cleaved antibody of from about 200 nM to about 400 nM. In some embodiments, the activatable anti-CTLA4 antibody is administered at a dose that provides a steady state concentration of the cleaved antibody of from about 300 nM to about 500 nM. In some embodiments, the activatable anti-CTLA4 antibody is administered at a dose that provides a steady state concentration of the cleaved antibody of from about 400 nM to about 600 nM. In some of the foregoing embodiments, the plasma concentration can be measured at the trough level of the anti-CTLA4 antibody (i.e., minimal concentration of each dosing cycle). For instance, the plasma concentration of a particular cycle can be measured immediately prior to administering a dose at the next cycle.

In some embodiments, the activatable anti-CTLA4 antibody is administered in combination with two or more therapeutic agents. In some embodiments, at least one of the therapeutic agents is an anti-PD1 antibody. In some such embodiments, the activatable anti-CTLA4 antibody is administered at a dose that provides a steady state concentration of the cleaved antibody of from about 50 nM to about 150 nM. In other such embodiments, the activatable anti-CTLA4 antibody is administered at a dose that provides a steady state concentration of the cleaved antibody of from about 50 nM to about 100 nM. In other such embodiments, the activatable anti-CTLA4 antibody is administered at a dose that provides a steady state concentration of the cleaved antibody of from about 50 nM to about 75 nM. In other such embodiments, the activatable anti-CTLA4 antibody is administered at a dose that provides a steady state concentration of the cleaved antibody of from about 75 nM to about 100 nM.

In any of the foregoing embodiments, the activatable anti-CTLA4 antibody (e.g., TY22404), when administered as a monotherapy or in combination with another agent, can be administered at a dose that provides a plasma concentration ratio of cleaved antibody to uncleaved antibody at steady state of from about 0.3 to about 1.0 at the trough level of a particular dosing cycle. In some embodiments, the activatable anti-CTLA4 antibody can be administered at a dose that provides a plasma concentration ratio of cleaved antibody to uncleaved antibody at steady state of from about 0.3 to about 1.0 at the trough level of a particular dosing cycle. In some embodiments, the activatable anti-CTLA4 antibody can be administered at a dose that provides a plasma concentration ratio of cleaved antibody to uncleaved antibody at steady state of from about 0.5 to about 0.8 at the trough level of a particular dosing cycle. In some embodiments, the activatable anti-CTLA4 antibody can be administered at a dose that provides a plasma concentration ratio of cleaved antibody to uncleaved antibody at steady state of from about 0.7 to about 1.0 at the trough level of a particular dosing cycle.

In any of the foregoing embodiments, the activatable anti-CTLA4 antibody can be administered to the subject at a dose of between about 3 mg/kg and about 20 mg/kg, e.g., about 3 mg/kg, about 6 mg/kg, about 10 mg/kg, about 15 mg/kg, or about 20 mg/kg. In some embodiments, the activatable antibody is administered between once every three to six weeks. In some such embodiments, the activatable anti-CTLA4 antibody is administered once every three weeks. In some such embodiments, the activatable anti-CTLA4 antibody is administered once every six weeks. In some embodiments, the activatable antibody is administered at a dose of about 10 mg/kg once every three weeks. In some embodiments, the activatable antibody is administered at a dose of about 20 mg/kg once every three weeks.

In some embodiments, the activatable anti-CTLA4 antibody is administered at a first higher dose (e.g., between about 10 mg/kg and about 20 mg) for at least one treatment cycle (as defined herein) followed by a lower dose (e.g., between about 3 mg/kg to about 10 mg/kg) in subsequent cycles. In one embodiment, the activatable anti-CTLA4 antibody is administered at a dose of about 20 mg/kg for at least one treatment cycle (e.g., one to three treatment cycles) and at a dose of about 10 mg/kg in subsequent treatment cycles (e.g., once every three weeks). In one embodiment, the activatable anti-CTLA4 antibody is administered at a dose of about 20 mg/kg for one treatment cycle and at a dose of about 10 mg/kg in subsequent treatment cycles (e.g., once every three weeks).

Based on noncompartmental analysis (NCA) and population PK modeling, the intact drug (i.e. uncleaved drug) shows dose-dependent increase in plasma. Surprisingly, however, body weight is not modeled to be a significant (e.g, exponent value >0.8 in allometric scaling as considered to be significant) covariate for key PK parameters such as clearance (CL) and volume of distribution (V). Virtual patient simulations indicate that flat dosing scheme is readily implementable either when the anti-CTLA4 as a monotherapy or as part of combination, as described below. In particular, in some embodiments, a flat dosing of the activatable anti-CTLA4 antibody (e.g., TY22404) can be from about 500 mg to about 1000 mg once every three weeks. In other embodiments, a flat dosing schedule of the activatable anti-CTLA4 antibody (e.g., TY22404) can be from about 700 mg to about 1000 mg once every three weeks. In other embodiments, a flat dosing schedule of the activatable anti-CTLA4 antibody (e.g., TY22404) can be from about 500 mg to about 750 mg once every three weeks. In other embodiments, a flat dosing schedule of the activatable anti-CTLA4 antibody (e.g., TY22404) can be from about 750 mg to about 1,000 mg once every three weeks. In other embodiments, a flat dosing schedule of the activatable anti-CTLA4 antibody (e.g., TY22404) can be from about 500 mg to about 1,200 mg once every six weeks. In other embodiments, a flat dosing schedule of the activatable anti-CTLA4 antibody (e.g., TY22404) can be from about 500 mg to about 1,000 mg once every six weeks. In other embodiments, a flat dosing schedule of the activatable anti-CTLA4 antibody (e.g., TY22404) can be from about 500 mg to about 750 mg once every six weeks. In other embodiments, a flat dosing schedule of the activatable anti-CTLA4 antibody (e.g., TY22404) can be from about 750 mg to about 1,000 mg once every six weeks. In other embodiments, the flat dosing schedule of the activatable anti-CTLA4 antibody (e.g., TY22404) can be about 500 mg once every three weeks or once every six weeks. In other embodiments, the flat dosing schedule of the activatable anti-CTLA4 antibody (e.g., TY22404) can be about 700 mg once every three weeks or once every six weeks. In other embodiments, the flat dosing schedule of the activatable anti-CTLA4 antibody (e.g., TY22404) can be about 1,000 mg once every three weeks or once every six weeks.

It is to be understood that one, some, or all of the properties of the various embodiments described above and herein may be combined to form other embodiments of the present application. These and other aspects of the present application will become apparent to one of skill in the art. These and other embodiments of the present application are further described by the detailed description that follows.

### BRIEF DESCRIPTION OF THE DRAWINGS

FIG. 1 shows CA125 Response in an Ovarian Serous Carcinoma Patient, with 90% reduction in CA125 from 303 to 31 U/ml (normal <35 U/ml) at the end of cycle 16/

FIG. 2 shows clinical Activity Assessment of TY22404 monotherapy. (A) Maximum tumor burden reduction in patients across dose level (0.1 to 20 mg/kg); (B) The duration of treatment in patients across dose level (0.1 to 20 mg/kg).

FIG. 3 shows plasma PK of total and cleaved TY22404 in the first 4 cycles in (A) 20 patients with PK data and (B) an ovarian serous carcinoma patient (case study). Total TY22404 and intact TY22404 was measured by LC-MS using signature peptides. Cleaved TY22404 is calculated as total TY22404 minus intact TY22404. C=treatment cycle

FIGS. 4A-4C shows efficacy of TY22404 monotherapy in a 39 years-old male patient with stage IIIB hepatocellular carcinoma. FIG. 4A shows increased ratio of Teff/Treg were observed in paired tumor biopsies from the patient. FIG. 4B shows Treg depletion was observed in paired tumor biopsies from the patient. FIG. 4C shows increased CD8+ T cells were observed in paired tumor biopsies from the patient.

FIGS. 5A-5B shows the response to TY22404 in combination with toripalimab in 18 evaluable patients* of three dose escalation cohorts who received TY22404 (6 mg/kg Q3W and 10 mg/kg Q3W or Q6W)+toripalimab (240 mg Q3W). FIG. 5A shows a swimmer plot. FIG. 5B shows a waterfall plot. *Evaluable patients with at least one valid post treatment tumor assessment. For the swimmer plot, bars end at the study day of end of treatment (EOT), last dose date, or the last tumor assessment, whichever is latest.

FIGS. 6A-6B show response to TY22404+toripalimab in selected gastrointestinal “cold tumors” including MSS CRC with liver metastasis at baseline and PDAC. FIG. 6A shows a swimmer plot. FIG. 6B shows a waterfall plot.

### DETAILED DESCRIPTION

### I. Definitions

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular. Generally, nomenclatures used in connection with, and techniques of, antibody engineering, immunotherapy, cell and tissue culture, molecular biology, immunology, microbiology, genetics and protein and nucleic acid chemistry described herein are those well-known and commonly used in the art.

The term “antibody” is used herein in the broadest sense and specifically covers monoclonal antibodies (including full length monoclonal antibodies), polyclonal antibodies, multispecific antibodies (e.g., bispecific antibodies, trispecific antibodies), and antibody fragments (e.g., Fab, Fab′, Fab′-SH, F(ab′)₂, Fv and/or a single-chain variable fragment or scFv) so long as they exhibit the desired biological activity.

The V_{H} and V_{L} regions can be further subdivided into regions of hypervariability, termed hyper-variable regions (HVR) based on structural and sequence analysis. HVRs are interspersed with regions that are more conserved, termed framework regions (FW) (see e.g., Chen et al. (1999) J. Mol. Biol. (1999) 293, 865-881). Each V_{H} and V_{L} is composed of three HVRs and four FWs, arranged from amino-terminus to carboxy-terminus in the following order: FW-1_HVR-1_FW-2_HVR-2_FW-3_HVR-3_FW4. Throughout the present application, the three HVRs of the heavy chain are referred to as HVR-H1, HVR-H2, and HVR-H3. Similarly, the three HVRs of the light chain are referred to as HVR-L1, HVR-L2, and HVR-L3.

As used herein, the term “CDR” or “complementarity determining region” is intended to mean the non-contiguous antigen combining sites found within the variable region of both heavy and light chain polypeptides. These particular regions have been described by Kabat et al., J. Biol. Chem. 252:6609-6616 (1977); Kabat et al., U.S. Dept. of Health and Human Services, “Sequences of proteins of immunological interest” (1991); Chothia et al., J. Mol. Biol. 196:901-917 (1987); Al-Lazikani B. et al., J. Mol. Biol., 273: 927-948 (1997); MacCallum et al., J. Mol. Biol. 262:732-745 (1996); Abhinandan and Martin, Mol. Immunol., 45: 3832-3839 (2008); Lefranc M. P. et al., Dev. Comp. Immunol., 27: 55-77 (2003); and Honegger and Pluckthun, J. Mol. Biol., 309:657-670 (2001), where the definitions include overlapping or subsets of amino acid residues when compared against each other.

The term “antigen-binding fragment” or “antigen binding portion” of an antibody refers to one or more portions of an antibody that retain the ability to bind to the antigen that the antibody bonds to (e.g., CTLA4).

The term “CTLA4” is used in the present application, and includes the human CTLA4 (e.g., UniProt accession number P16410), as well as variants, isoforms, and species homologs thereof (e.g., mouse CTLA4 (UniProt accession number P09793), rat CTLA4 (UniProt accession number Q9Z1A7), dog CTLA4 (UniProt accession number Q9XS11), cynomolgus monkey CTLA4 (UniProt accession number G7PL88), etc.). Accordingly, an anti-CTLA4 antibody (e.g., an activatable antibody) as defined and disclosed herein, may also bind CTLA4 from species other than human. In other cases, an anti-CTLA4 antibody may be completely specific for the human CTLA4 and may not exhibit species or other types of cross-reactivity.

The term “CTLA4 antibody” refers to an antibody, as defined herein, capable of binding to human CTLA4 (e.g., an activatable anti-CTLA4 antibody).

The term “epitope” refers to a part of an antigen to which an antibody (or antigen-binding fragment thereof) binds. Epitopes can be formed both from contiguous amino acids or noncontiguous amino acids juxtaposed by tertiary folding of a protein.

A “human antibody” is one which possesses an amino acid sequence which corresponds to that of an antibody produced by a human or a human cell or derived from a non-human source that utilizes human antibody repertoires or other human antibody-encoding sequences.

The term “humanized antibody” refers to a chimeric antibody that contains amino acid residues derived from human antibody sequences.

The term “illustrative antibody” refers to any one of the antibodies described in the disclosure and designated as those listed in Tables A and B, and any antibodies comprising the 6 HVRs and/or the VH and VLs of the antibodies listed in Tables A and B. These antibodies may be in any class (e.g., IgA, IgD, IgE, IgG, and IgM).

The term “mammal” refers to any animal species of the Mammalia class.

As used herein, “sequence identity” between two polypeptide sequences indicates the percentage of amino acids that are identical between the sequences.

The term “treat”, “treating”, or “treatment”, with reference to a certain disease condition in a mammal, refers causing a desirable or beneficial effect in the mammal having the disease condition. The desirable or beneficial effect may include reduced frequency or severity of one or more symptoms of the disease (i.e., tumor growth and/or metastasis, or other effect mediated by the numbers and/or activity of immune cells, and the like), or arrest or inhibition of further development of the disease, condition, or disorder. In the context of treating cancer in a mammal, the desirable or beneficial effect may include inhibition of further growth or spread of cancer cells, death of cancer cells, inhibition of reoccurrence of cancer, reduction of pain associated with the cancer, or improved survival of the mammal. The effect can be either subjective or objective. For example, if the mammal is human, the human may note improved vigor or vitality or decreased pain as subjective symptoms of improvement or response to therapy. Alternatively, the clinician may notice a decrease in tumor size or tumor burden based on physical exam, laboratory parameters, tumor markers or radiographic findings. Some laboratory signs that the clinician may observe for response to treatment include normalization of tests, such as white blood cell count, red blood cell count, platelet count, erythrocyte sedimentation rate, and various enzyme levels. Additionally, the clinician may observe a decrease in a detectable tumor marker. Alternatively, other tests can be used to evaluate objective improvement, such as sonograms, nuclear magnetic resonance testing and positron emissions testing.

The term “prevent” or “preventing,” with reference to a certain disease condition in a mammal, refers to preventing or delaying the onset of the disease, or preventing the manifestation of clinical or subclinical symptoms thereof.

As used herein, a “subject”, “patient”, or “individual” may refer to a human or a non-human animal. A “non-human animal” may refer to any animal not classified as a human, such as domestic, farm, or zoo animals, sports, pet animals (such as dogs, horses, cats, cows, etc.), as well as animals used in research. Research animals may refer without limitation to nematodes, arthropods, vertebrates, mammals, frogs, rodents (e.g., mice or rats), fish (e.g., zebrafish or pufferfish), birds (e.g., chickens), dogs, cats, and non-human primates (e.g., rhesus monkeys, cynomolgus monkeys, chimpanzees, etc.). In some embodiments, the subject, patient, or individual is a human.

An “effective amount” refers to at least an amount effective, at dosages and for periods of time necessary, to achieve one or more desired or indicated effects, including a therapeutic or prophylactic result. An effective amount can be provided in one or more administrations. For purposes of the present application, an effective amount of antibody, drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition (e.g., an effective amount as administered as a monotherapy or combination therapy). Thus, an “effective amount” may be considered in the context of administering one or more therapeutic agents, and a single agent may be considered to be given in an effective amount if, in conjunction with one or more other agents, a desirable result may be or is achieved.

The terms “recurrence,” “relapse” or “relapsed” refers to the return of a cancer or disease after clinical assessment of the disappearance of disease. A diagnosis of distant metastasis or local recurrence can be considered a relapse.

The term “refractory” or “resistant” refers to a cancer or disease that has not responded to treatment.

As used herein, “complete response” or “CR” refers to disappearance of all target lesions; “partial response” or “PR” refers to at least a 30% decrease in the sum of the longest diameters (SLD) of target lesions, taking as reference the baseline SLD; and “stable disease” or “SD” refers to neither sufficient shrinkage of target lesions to qualify for PR, nor sufficient increase to qualify for PD, taking as reference the smallest SLD since the treatment started.

As used herein, “progressive disease” or “PD” refers to at least a 20% increase in the SLD of target lesions, taking as reference the smallest SLD recorded since the treatment started or the presence of one or more new lesions.

As used herein, “progression free survival” (PFS) refers to the length of time during and after treatment during which the disease being treated (e.g., cancer) does not get worse. Progression-free survival may include the amount of time patients have experienced a complete response or a partial response, as well as the amount of time patients have experienced stable disease.

As used herein, “overall response rate” (ORR) refers to the sum of complete response (CR) rate and partial response (PR) rate.

As used herein, “overall survival” refers to the percentage of individuals in a group who are likely to be alive after a particular duration of time.

As used herein, a “baseline level” or “baseline value” refers to a level or a value of a subject before the subject begins a treatment, such as an anti-CTLA4 antibody treatment.

A “reference sample”, “reference cell”, “reference tissue”, “control sample”, “control cell”, or “control tissue”, as used herein, refers to a sample, cell, tissue, standard, or level that is used for comparison purposes.

By “correlate” or “correlating” is meant comparing, in any way, the performance and/or results of a first analysis or protocol with the performance and/or results of a second analysis or protocol. For example, one may use the results of a first analysis or protocol in carrying out a second protocols and/or one may use the results of a first analysis or protocol to determine whether a second analysis or protocol should be performed.

An “effective response” of a patient or a patient's “responsiveness” to treatment with a medicament and similar wording refers to the clinical or therapeutic benefit imparted to a patient at risk for, or suffering from, a disease or disorder, such as cancer. In one embodiment, such benefit includes any one or more of: extending survival (including overall survival and progression free survival); resulting in an objective response (including a complete response or a partial response); or improving signs or symptoms of cancer.

A patient who “does not have an effective response” to treatment refers to a patient who does not have any one of extending survival (including overall survival and progression free survival); resulting in an objective response (including a complete response or a partial response); or improving signs or symptoms of cancer.

As used herein, the twenty conventional amino acids and their abbreviations follow conventional usage. See Immunology—A Synthesis (2nd Edition, E. S. Golub and D. R. Gren, Eds., Sinauer Associates, Sunderland, Mass. (1991)).

As used herein, the singular forms “a”, “an” and “the” include plural referents unless the content clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the present application described herein include “comprising,” “consisting,” and “consisting essentially of” aspects and embodiments.

As used herein, reference to “not” a value or parameter generally means and describes “other than” a value or parameter. For example, the method is not used to treat cancer of type X means the method is used to treat cancer of types other than X.

The term “about X-Y” used herein has the same meaning as “about X to about Y.”

The term “and/or” as used herein a phrase such as “A and/or B” is intended to include both A and B; A or B; A (alone); and B (alone). Likewise, the term “and/or” as used herein a phrase such as “A, B, and/or C” is intended to encompass each of the following embodiments: A, B, and C; A, B, or C; A or C; A or B; B or C; A and C; A and B; B and C; A (alone); B (alone); and C (alone).

### II. Methods of Treatment

The present application provides methods for treating cancers in a subject using an activatable anti-CTLA4 antibody that specifically binds to human CTLA4. The activatable anti-CTLA4 antibody may be administered alone as monotherapy, or administered in combination with one or more additional therapeutic agents or therapies.

The methods described herein are useful for treating a variety of cancers. In some embodiments, the cancer is a solid cancer. In some embodiments, the cancer is a liquid cancer. A variety of cancers where CTLA4 is implicated, whether malignant or benign and whether primary or secondary, may be treated or prevented with a method provided by the disclosure. Exemplary cancers include, but are not limited to, liver cancer, a cancer of the digestive system (e.g., colon cancer, colorectal cancer (CRC), gastrointestinal stroma tumor (GIST), cecum adenocarcinoma), lung cancer, bone cancer, heart cancer, brain cancer, kidney cancer, bladder cancer, a hematological cancer (e.g., leukemia), skin cancer, breast cancer, thyroid cancer, neuroendocrine cancer, pancreatic cancer (e.g., pancreatic ductal adenocarcinoma (PDAC)), a head and/or neck cancer, an eye-related cancer, a male reproductive system cancer (e.g., prostate cancer, testicular cancer), or a female reproductive system cancer (e.g., uterine cancer, cervical cancer (e.g., cervical squamous cell carcinoma and endocervical adenocarcinoma (CESC), endometrial carcinoma, endometrium cancer). In some embodiments, the cancer is squamous cell carcinoma (SCC) (e.g., anal, anorectal, penile, or cutaneous). In some embodiments, the cancer is anal squamous cell carcinoma or penile squamous cell carcinoma. In some embodiments, the cancer is adenocarcinoma. In some embodiments, the cancer is kidney cancer, such as renal cell carcinoma, or urothelial carcinoma. In some embodiments, the cancer is ovarian cancer ((e.g., ovarian serous cystadenocarcinoma (OV), pancreatic cancer, cholangiocarcinoma, lung cancer (e.g., NSCLC), breast cancer, melanoma, hepatocellular carcinoma, glioblastoma, renal cell carcinoma, head and neck squamous cell carcinoma, or colorectal cancer (e.g., MSI-H or dMMR+colorectal cancer, microsatellite stable (MSS) colorectal cancer). In some embodiments, the cancer is melanoma, NSCLC, hepatocellular carcinoma, renal cell carcinoma, head and neck squamous cell carcinoma, pancreatic cancer, anal squamous cell carcinoma, penile squamous cell carcinoma or colorectal cancer (e.g., MSI-H or dMMR+colorectal cancer, microsatellite stable (MSS) colorectal cancer).

In some embodiments, the activatable anti-CTLA4 is administered to a cancer patient as a monotherapy. In some embodiments, the activatable anti-CTLA4 is administered to a cancer patient as a combination therapy. In some embodiments, the cancer is squamous cell carcinoma (e.g., anal, anorectal, penile, or cutaneous). In some embodiments, the cancer is adenocarcinoma. In some embodiments, the cancer is melanoma. In other embodiments, the cancer is ovarian cancer, pancreatic cancer, cholangiocarcinoma, lung cancer, breast cancer, hepatocellular carcinoma, glioblastoma, renal cell carcinoma, head and neck squamous cell carcinoma, colorectal cancer, gastrointestinal stroma tumor (GIST), endometrial carcinoma. In some embodiments, the lung cancer is non-small cell lung cancer (NSCLC). In some embodiments, the colorectal cancer is MSS CRC. In some embodiments, the cancer is anal squamous cell carcinoma or penile squamous cell carcinoma.

Cancer treatments can be evaluated by, e.g., tumor regression, tumor weight or size shrinkage, time to progression, duration of survival, progression free survival, overall response rate, duration of response, quality of life, protein expression and/or activity. Approaches to determining efficacy of therapy can be employed, including for example, measurement of response through radiological imaging.

The activatable anti-CTLA4 antibodies and one or more additional therapeutic agents, and compositions provided by the present disclosure can be administered via any suitable enteral route or parenteral route of administration. The term “enteral route” of administration refers to the administration via any part of the gastrointestinal tract. Examples of enteral routes include oral, mucosal, buccal, and rectal route, or intragastric route. “Parenteral route” of administration refers to a route of administration other than enteral route. Examples of parenteral routes of administration include intravenous, intramuscular, intradermal, intraperitoneal, intratumor, intravesical, intraarterial, intrathecal, intracapsular, intraorbital, intracardiac, transtracheal, intraarticular, subcapsular, subarachnoid, intraspinal, epidural and intrasternal, subcutaneous, or topical administration. The antibodies and compositions of the disclosure can be administered using any suitable method, such as by oral ingestion, nasogastric tube, gastrostomy tube, injection, infusion, implantable infusion pump, and osmotic pump. The suitable route and method of administration may vary depending on a number of factors such as the specific antibody being used, the rate of absorption desired, specific formulation or dosage form used, type or severity of the disorder being treated, the specific site of action, and conditions of the patient, and can be readily selected by a person skilled in the art. In some embodiments, the anti-CTLA4 antibody is administered intravenously.

The effective amount of the activatable anti-CTLA4 antibody and/or one or more additional therapeutic agents may be administered in a single dose or in multiple doses. For methods that comprises administration of the anti-CTLA4 antibody in multiple doses, exemplary dosing frequencies include, but are not limited to, weekly, weekly without break, weekly for two out of three weeks, weekly for three out of four weeks, once every three weeks, once every two weeks, monthly, every six months, yearly, etc. In some embodiments, the anti-CTLA4 antibody is administered about weekly, once every 2 weeks, once every 3 weeks, once every 6 weeks, or once every 12 weeks. In some embodiments, the intervals between each administration are less than about any of 3 years, 2 years, 12 months, 11 months, 10 months, 9 months, 8 months, 7 months, 6 months, 5 months, 4 months, 3 months, 2 months, 1 month, 4 weeks, 3 weeks, 2 weeks, or 1 week. In some embodiments, the intervals between each administration are more than about any of 1 week, 2 weeks, 3 weeks, 4 weeks, 1 month, 2 months, 3 months, 4 months, 5 months, 6 months, 7 months, 8 months, 9 months, 10 months, 11 months, 12 months, 2 years, or 3 years. In some embodiments, there is no break in the dosing schedule.

In some embodiments, the activatable anti-CTLA4 antibody and/or one or more additional therapeutic agents is administered at a low frequency, for example, any one of no more frequent than once per week, once every other week, once per three weeks, once per month, once per 2 months, once per 3 months, once per 4 months, once per 5 months, once per 6 months, once per 7 months, once per 8 months, once per 9 months, once per 10 months, once per 11 months, once per year, or less. In some embodiments, the anti-CTLA4 antibody and/or one or more additional therapeutic agents is administered in a single dose. In some embodiments, the activatable anti-CTLA4 antibody and/or one or more additional therapeutic agents is administered about once every three weeks. In some embodiments, the activatable anti-CTLA4 antibody and/or one or more additional therapeutic agents is administered about once every six weeks.

In some embodiments, the activatable anti-CTLA4 antibody and/or one or more additional therapeutic agents is administered for 2 or more cycles, such as about any one of 2, 3, 4, 5, 6, 7, 8, 9, 10, 11, 12, or more cycles. In some embodiments, the anti-CTLA4 antibody is administered for at least 4 cycles.

In some embodiments, the treatment comprises an initial phase and a subsequent maintenance phase. In some embodiments, the activatable anti-CTLA4 antibody and/or one or more additional therapeutic agents is administered less frequently in the maintenance phase than in the initial phase. In some embodiments, the anti-CTLA4 antibody and/or one or more additional therapeutic agents is administered at the same frequently in the maintenance phase as in the initial phase. In some embodiments, the treatment comprises an initial phase wherein the anti-CTLA4 antibody and/or one or more additional therapeutic agents is administered about once every three weeks for at least 4 cycles, and a maintenance phase wherein the anti-CTLA4 antibody and/or one or more additional therapeutic agents is administered about once every 4 weeks to once every 12 weeks, such as once every 4 weeks, once every 6 weeks, once every 8 weeks, once every 10 weeks, or once every 12 weeks. In some embodiments, the dosing frequency in the maintenance phase is adjusted depending on one or more biomarkers, such as T_{reg} cells, CD8+Tₑₘ cells, CD4+Tₑₘ cells, a ratio of CD8+Tₑₘ cells to T_{reg} cells, a ratio of CD4+Tₑₘ cells to T_{reg} cells, CD25+ T cells, and/or NK cells. For example, if the subject shows an increase in the ratio of CD8+Tₑₘ cells to T_{reg} cells after receiving the anti-CTLA4 antibody, the subject may be further administered an anti-CTLA4 antibody at about every 4 weeks.

The administration of the anti-CTLA4 antibody and/or one or more additional therapeutic agents can be extended over an extended period of time, such as from about a week to about a month, from about a month to about a year, from about a year to about several years. In some embodiments, the anti-CTLA4 antibody and/or one or more additional therapeutic agents is administered over a period of at least any of about 1 week, 2 weeks, 3 weeks, 4 weeks, 5 weeks, 1 month, 2 months, 3 months, 4 months, 5 months, 6 months, 7 months, 8 months, 9 months, 10 months, 11 months, 12 months, 1 year, 2 years, 3 years, 4 years, or more.

In some embodiments where the activatable anti-CTLA4 antibody, upon cleavage of the MM, comprises a heavy chain variable region comprising the amino acid sequence of SEQ ID NO: 87 or an amino acid sequence having at least 90% (e.g., at least 92%, at least 93%, at least 94%, at least 95%, at least 96%, at least 97%, at least 98% or at least 99%) sequence identity to the amino acid sequence of SEQ ID NO: 87, and/or a light chain variable region comprising the amino acid sequence of SEQ ID NO: 100 or an amino acid sequence having at least 90% (e.g., at least 92%, at least 93%, at least 94%, at least 95%, at least 96%, at least 97%, at least 98% or at least 99%) sequence identity to the amino acid sequence of SEQ ID NO: 100, the activatable anti-CTLA4 antibody is administered at a dose of between about 3 mg/kg to about 20 mg/kg once every three weeks, about 3 mg/kg to about 15 mg/kg once every three weeks, about 6 mg/kg to about 15 mg/kg once every three weeks, or from about 6 mg/kg to about 10 mg/kg once every three weeks. In some such embodiments, the activatable anti-CTLA4 antibody is administered at a dose of about 3 mg/kg once every three weeks. In other such embodiments, the activatable anti-CTLA4 antibody is administered at a dose of about 6 mg/kg once every three weeks. In other such embodiments, the activatable anti-CTLA4 antibody is administered at a dose of about 10 mg/kg once every three weeks. In some of the foregoing embodiments, the activatable antibody comprises an HVR-H1 comprising the amino acid sequence of SEQ ID NO: 23, an HVR-H2 comprising the amino acid sequence of SEQ ID NO: 35, an HVR-H3 comprising the amino acid sequence of SEQ ID NO: 45, an HVR-L1 comprising the amino acid sequence of SEQ ID NO: 58, an HVR-L2 comprising the amino acid sequence of SEQ ID NO: 66, and an HVR-L3 comprising the amino acid sequence of SEQ ID NO: 75. In some of the foregoing embodiments, the activatable anti-CTLA4 antibody comprises a human IgG1 Fc region, such as a wild type IgG1 Fc region or a variant that has enhanced ADCC activity. In some of the foregoing embodiments, the activatable anti-CTLA4 antibody comprises an MM amino acid sequence selected from the group consisting of SEQ ID NOS: 189-196. In some of the foregoing embodiments, the activatable anti-CTLA4 antibody comprises a MM amino acid sequence selected from the group consisting of SEQ ID NOS: 213-216. In some of the foregoing embodiments, the activatable anti-CTLA4 antibody comprises a MM amino acid sequence selected from the group consisting of SEQ ID NOS: 141-147. In some of the foregoing embodiments, the activatable anti-CTLA4 antibody comprises a MM amino acid sequence of SEQ ID NO: 200. In some of the foregoing embodiments, the activatable anti-CTLA4 antibody comprises a heavy chain comprising the amino acid sequence of SEQ ID NO:320 or 321, or an amino acid sequence having at least 90% (e.g., at least 92%, at least 93%, at least 94%, at least 95%, at least 96%, at least 97%, at least 98% or at least 99%) sequence identity to the amino acid sequence of SEQ ID NO: 320 or 321, and a light chain comprising the amino acid sequence of SEQ ID NO:322, or an amino acid sequence having at least 90% (e.g., at least 92%, at least 93%, at least 94%, at least 95%, at least 96%, at least 97%, at least 98% or at least 99%) sequence identity to the amino acid sequence of SEQ ID NO: 322. In some of the foregoing embodiments, the activatable anti CTLA4 antibody is administered to a patient with melanoma, non-small cell lung cancer, renal cell carcinoma, or hepatocellular carcinoma. In other of the foregoing embodiments, the activatable anti CTLA4 antibody is administered to a patient with a MSI-H or dMMR cancer. In other of the foregoing embodiments, the activatable anti CTLA4 antibody is administered to a patient with a cancer that has metastasized. In some of the foregoing embodiments, the activatable anti-CTLA4 antibody is administered to a patient that is resistant or refractory to prior cancer therapy, including other anti-CTLA4 antibodies, anti-PD-1 antibodies, anti PD-L1 antibodies, or combinations thereof.

In some embodiments where the activatable anti-CTLA4 antibody, upon cleavage of the MM, comprises a heavy chain variable region comprising the amino acid sequence of SEQ ID NO: 87 or an amino acid sequence having at least 90% (e.g., at least 92%, at least 93%, at least 94%, at least 95%, at least 96%, at least 97%, at least 98% or at least 99%) sequence identity to the amino acid sequence of SEQ ID NO: 87, and/or a light chain variable region comprising the amino acid sequence of SEQ ID NO: 100 or an amino acid sequence having at least 90% (e.g., at least 92%, at least 93%, at least 94%, at least 95%, at least 96%, at least 97%, at least 98% or at least 99%) sequence identity to the amino acid sequence of SEQ ID NO: 100, the activatable anti-CTLA4 antibody is administered at a first higher dose (e.g., between about 10 mg/kg and about 20 mg) for at least one treatment cycle (as defined herein) followed by a lower dose (e.g., between about 3 mg/kg to about 10 mg/kg) in subsequent cycles. In some such embodiments, the activatable anti-CTLA4 antibody is administered at a dose of about 10 mg/kg for at least one treatment cycle (e.g., one to three treatment cycles) and at a dose of about 6 mg/kg in subsequent treatment cycles. In other such embodiments, the activatable anti-CTLA4 antibody is administered at a dose of about 10 mg/kg for at least one treatment cycle (e.g., one to three treatment cycles) and at a dose of about 3 mg/kg in subsequent treatment cycles. In other such embodiments, the activatable anti-CTLA4 antibody is administered at a dose of about 15 mg/kg for at least one treatment cycle (e.g., one to three treatment cycles) and at a dose of about 10 mg/kg in subsequent treatment cycles. In other such embodiments, the activatable anti-CTLA4 antibody is administered at a dose of about 20 mg/kg for at least one treatment cycle (e.g., one to three treatment cycles) and at a dose of about 10 mg/kg in subsequent treatment cycles. In some of the foregoing embodiments, the activatable antibody comprises an HVR-H1 comprising the amino acid sequence of SEQ ID NO: 23, an HVR-H2 comprising the amino acid sequence of SEQ ID NO: 35, an HVR-H3 comprising the amino acid sequence of SEQ ID NO: 45, an HVR-L1 comprising the amino acid sequence of SEQ ID NO: 58, an HVR-L2 comprising the amino acid sequence of SEQ ID NO: 66, and an HVR-L3 comprising the amino acid sequence of SEQ ID NO: 75. In some of the foregoing embodiments, the activatable anti-CTLA4 antibody comprises a heavy chain comprising the amino acid sequence of SEQ ID NO:320 or 321, or an amino acid sequence having at least 90% (e.g., at least 92%, at least 93%, at least 94%, at least 95%, at least 96%, at least 97%, at least 98% or at least 99%) sequence identity to the amino acid sequence of SEQ ID NO: 320 or 321, and a light chain comprising the amino acid sequence of SEQ ID NO:322, or an amino acid sequence having at least 90% (e.g., at least 92%, at least 93%, at least 94%, at least 95%, at least 96%, at least 97%, at least 98% or at least 99%) sequence identity to the amino acid sequence of SEQ ID NO: 322. In some of the foregoing embodiments, the activatable anti CTLA4 antibody is administered to a patient with melanoma, non-small cell lung cancer, renal cell carcinoma, or hepatocellular carcinoma. In other of the foregoing embodiments, the activatable anti CTLA4 antibody is administered to a patient with a MSI-H or dMMR cancer. In other of the foregoing embodiments, the activatable anti CTLA4 antibody is administered to a patient with a cancer that has metastasized. In some of the foregoing embodiments, the activatable anti-CTLA4 antibody is administered to a patient that is resistant or refractory to prior cancer therapy, including other anti-CTLA4 antibodies, anti-PD-1 antibodies, anti PD-L1 antibodies, or combinations thereof.

In some embodiments, the activatable anti-CTLA4 antibody is administered at a dose of about 20 mg/kg for one treatment cycle and at a dose of about 10 mg/kg in subsequent treatment cycles (e.g., once every three weeks). For instance, in one embodiment, the anti-CTLA4 antibody is administered at an initial (loading) dose of 20 mg/kg and then is administered additional doses (maintenance doses) of 10 mg/kg every three weeks. The start of the maintenance doses can be at a predetermined time following administration of the loading dose. For instance, in some embodiments, the first maintenance dose can be administered three weeks after administration of the loading dose.

It has been found that administration of a single loading dose followed by subsequent administration of maintenance doses, as set forth above, results in establishing a steady state concentration of the cleaved antibody more rapidly than when the activatable antibody is administered without a loading dose. For instance, in some embodiments, a steady state concentration of the cleaved antibody can be established within 1 week, within 2 weeks, within 3 week, within 4 weeks, within 5 week, within 6 weeks or within 7 weeks following the initial administration of the loading dose.

In any of the foregoing, the activatable anti-CTLA4 antibody is administered at a dose that provides a steady state concentration of the cleaved antibody of from about 100 nM to about 200 nM. In some embodiments, the activatable anti-CTLA4 antibody is administered at a dose that provides a steady state concentration of the cleaved antibody of from about 125 nM to about 200 nM. In some embodiments, the activatable anti-CTLA4 antibody is administered at a dose that provides a steady state concentration of the cleaved antibody of from about 100 nM to about 175 nM. In some embodiments, the activatable anti-CTLA4 antibody is administered at a dose that provides a steady state concentration of the cleaved antibody of from about 125 nM to about 175 nM. In some embodiments, the activatable anti-CTLA4 antibody is administered at a dose that provides a steady state concentration of the cleaved antibody of from about 100 nM to about 150 nM. In some embodiments, the activatable anti-CTLA4 antibody is administered at a dose that provides a steady state concentration of the cleaved antibody of from about 125 nM to about 150 nM. In some embodiments, the activatable anti-CTLA4 antibody is administered at a dose that provides a steady state concentration of the cleaved antibody of from about 150 nM to about 200 nM. In some embodiments, the activatable anti-CTLA4 antibody is administered at a dose that provides a steady state concentration of the cleaved antibody of from about 200 nM to about 600 nM. In some embodiments, the activatable anti-CTLA4 antibody is administered at a dose that provides a steady state concentration of the cleaved antibody of from about 200 nM to about 400 nM. In some embodiments, the activatable anti-CTLA4 antibody is administered at a dose that provides a steady state concentration of the cleaved antibody of from about 300 nM to about 500 nM. In some embodiments, the activatable anti-CTLA4 antibody is administered at a dose that provides a steady state concentration of the cleaved antibody of from about 400 nM to about 600 nM. In some of the foregoing embodiments, the plasma concentration can be measured at the trough level of the anti-CTLA4 antibody (i.e., minimal concentration of each dosing cycle). For instance, the plasma concentration of a particular cycle can be measured immediately prior to administering a dose at the next cycle.

In some embodiments, the activatable anti-CTLA4 antibody is administered in combination with two or more therapeutic agents. In some embodiments, at least one of the therapeutic agents is an anti-PD1 antibody. In some such embodiments, the activatable anti-CTLA4 antibody is administered at a dose that provides a steady state concentration of the cleaved antibody of from about 50 nM to about 150 nM. In other such embodiments, the activatable anti-CTLA4 antibody is administered at a dose that provides a steady state concentration of the cleaved antibody of from about 50 nM to about 100 nM. In other such embodiments, the activatable anti-CTLA4 antibody is administered at a dose that provides a steady state concentration of the cleaved antibody of from about 50 nM to about 75 nM. In other such embodiments, the activatable anti-CTLA4 antibody is administered at a dose that provides a steady state concentration of the cleaved antibody of from about 75 nM to about 100 nM.

In any of the embodiments of the preceding two paragraphs, the anti-CTLA4 antibody can be administered as a monotherapy or in combination with one or more anticancer agents, as disclosed herein. For instance, the anti-CTLA4 antibody can be administered in combination with an anti-PD-1 antibody. In some embodiments, the combination of the anti-CTLA4 antibody and the anti-PD-1 antibody displays a synergistic effect.

In some embodiments, treatment with an anti-CTLA4 antibody and/or one or more additional therapeutic agents depletes Treg cells in tumors. In some embodiments, treatment with an anti-CTLA4 antibody and/or one or more additional therapeutic agents does not deplete Treg cells in peripheral tissues.

In some embodiments, the subject has been previously treated with a prior therapy. In some embodiments, the subject has previously received any one of 1, 2, 3, 4, or more prior therapies. In some embodiments, the subject has exhausted all other available therapies. In some embodiments, the subject is unresponsive or resistant to a prior therapy. In some embodiments, the subject has disease reoccurrence subsequent to a prior therapy. In some embodiments, the subject is refractory to a prior therapy. In some embodiments, the subject has failed a prior therapy within about 1 year, 6 months, 3 months or less. In some embodiments, the subject has not previously received a prior therapy.

In some embodiments, the subject has been previously treated with a standard therapy for the cancer. In some embodiments, the subject is unresponsive or resistant to a standard therapy. In some embodiments, the subject has disease reoccurrence subsequent to a standard therapy. In some embodiments, the subject is refractory to a standard therapy. In some embodiments, the subject has failed a standard therapy within about 1 year, 6 months, 3 months or less. In some embodiments, the subject has not previously received a standard therapy. In some embodiments, the subject has refused or is ineligible for a standard therapy.

In some embodiments, the prior therapy (e.g., standard therapy) is selected from the group consisting of viral gene therapy, immunotherapy, targeted therapy, radiation therapy, and chemotherapy. In some embodiments, the prior therapy is an immune checkpoint inhibitor. In some embodiments, the prior therapy is an inhibitor of CTLA4, PD-1, or a PD-1 ligand (e.g., PD-L1 or PD-L2). In some embodiments, the prior therapy is an inhibitor of CTLA4, such as an anti-CTLA4 antibody that is different from the anti-CTLA4 antibodies described herein. In some embodiments, the prior therapy is ipilimumab. In some embodiments, the prior therapy is ipilimumab and/or nivolumab.

In some embodiments, the prior therapy is an inhibitor of PD-1 or a PD-1 ligand, including a PD-1 binding antagonist, a PDL1 binding antagonist and a PDL2 binding antagonist. Alternative names for “PD-1” include CD279 and SLEB2. Alternative names for “PDL1” include B7-H1, B7-4, CD274, and B7-H. Alternative names for “PDL2” include B7-DC, Btdc, and CD273. In some embodiments, PD-1, PDL1, and PDL2 are human PD-1, PDL1 and PDL2.

In some embodiments, the inhibitor of PD-1 is a molecule that inhibits the binding of PD-1 to its ligand binding partners. In some embodiments, the inhibitor of a PD-1 ligand is an inhibitor of PD-L1 and/or PD-L2. In some embodiments, the inhibitor of PD-L1 is a molecule that inhibits the binding of PDL1 to its binding partners. In some embodiments, a PD-L2 binding partner is PD-1 and/or B7-1. In some embodiments, the inhibitor of a PD-1 ligand is a molecule that inhibits the binding of PD-L2 to its binding partners. In some embodiments, a PD-L2 binding partner is PD-1. The inhibitor may be an antibody, an antigen binding fragment thereof, an immunoadhesin, a fusion protein, or oligopeptide.

In some embodiments, the inhibitor of PD-1 is an anti-PD-1 antibody (e.g., a human antibody, a humanized antibody, or a chimeric antibody). In some embodiments, the anti-PD-1 antibody is selected from the group consisting of nivolumab and CT-011. In some embodiments, the inhibitor of PD-1 is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the inhibitor of PD-1 is AMP-224. Nivolumab, also known as MDX-1106-04, MDX-1106, ONO-4538, BMS-936558, and OPDIVO®, is an anti-PD-1 antibody described in WO2006/121168. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342. In some embodiments, the anti-PD-1 antibody is nivolumab (CAS Registry Number: 946414-94-4).

Prior therapies (e.g., standard therapies) also encompass surgery to remove a tumor and radiation therapy. Exemplary radiation therapies include, but are not limited to, ionizing (electromagnetic) radiotherapy (e.g., X-rays or gamma rays) and particle beam radiation therapy (e.g., high linear energy radiation). The source of radiation can be external or internal to the subject.

The methods described herein are useful for various aspects of cancer treatment. In some embodiments, there is provided a method of inhibiting cell proliferation (such as tumor growth) in an individual, comprising administering to the individual an effective amount of any one of the anti-CTLA4 antibodies described herein. In some embodiments, at least about 10% (including for example at least about any of 20%, 30%, 40%, 60%, 70%, 80%, 90%, 95% or more) cell proliferation is inhibited.

In some embodiments, there is provided a method of inhibiting tumor metastasis in an individual, comprising administering to the individual an effective amount of any one of the anti-CTLA4 antibodies described herein. In some embodiments, at least about 10% (including for example at least about any of 20%, 30%, 40%, 60%, 70%, 80%, 90%, 95% or more) metastasis is inhibited.

In some embodiments, there is provided a method of reducing (such as eradicating) pre-existing tumor metastasis (such as metastasis to the lymph node) in an individual, comprising administering to the individual an effective amount of any one of the anti-CTLA4 antibodies described herein. In some embodiments, at least about 10% (including for example at least about any of 20%, 30%, 40%, 60%, 70%, 80%, 90%, 95% or more) metastasis is reduced.

In some embodiments, there is provided a method of reducing incidence or burden of preexisting tumor metastasis (such as metastasis to the lymph node) in an individual, comprising administering to the individual an effective amount of any one of the anti-CTLA4 antibodies described herein.

In some embodiments, there is provided a method of reducing tumor size in an individual, comprising administering to the individual an effective amount of any one of the anti-CTLA4 antibodies described herein. In some embodiments, the method reduces tumor size by at least about 10% (including for example at least about any of 20%, 30%, 40%, 60%, 70%, 80%, 90%, 95% or more).

In some embodiments, there is provided a method of prolonging time to disease progression of cancer in an individual, comprising administering to the individual an effective amount of any one of the anti-CTLA4 antibodies described herein. In some embodiments, the method prolongs the time to disease progression by at least any of 1, 2, 3, 4, 5, 6, 7, 8, 9, 10, 11, 12, 16, 20, 24, 28, 32, 36, or more weeks.

In some embodiments, there is provided a method of prolonging survival (e.g., overall survival or progression-free survival) of an individual having cancer, comprising administering to the individual an effective amount of any one of the anti-CTLA4 antibodies described herein. In some embodiments, the method prolongs the survival of the individual by at least any of 1, 2, 3, 4, 5, 6, 7, 8, 9, 10, 11, 12, 18, or 24 months.

In some embodiments, there is provided a method of alleviating one or more symptoms in an individual having cancer, comprising administering to the individual an effective amount of any one of the anti-CTLA4 antibodies described herein. In some embodiments, the anti-CTLA4 antibody is administered in combination with one or more additional therapeutic agents.

In some embodiments, there is provided a method of improving the quality of life in an individual having cancer, comprising administering to the individual an effective amount of any one of the anti-CTLA4 antibodies described herein.

### Iii. Activatable Binding Polypeptides Targeting CTLA4

The present disclosure also relates, in part, to precision/context-dependent activatable binding polypeptides (i.e., activatable antibodies) that bind to human CTLA4, including activatable antibodies comprising any of the anti-CTLA4 antibodies described herein (e.g., anti-CTLA4 antibodies, anti-CTLA4 antibody binding fragments, and/or anti-CTLA4 antibody derivatives), antigen binding fragments of the activatable anti-CTLA4 antibodies, and/or derivatives of the activatable anti-CTLA4 antibodies. In some embodiments, the activatable anti-CTLA4 antibodies described herein may have improved safety profiles. For example, the anti-CTLA4 antibodies described herein may have better safety margin as assessed by spleen weight change. The change in spleen size with the increase in drug dose administered is used as a benchmark to assess the safety margin of the drug candidate used. The activatable anti-CTLA4 antibodies described herein have a better safety margin relative to the parental antibody (the antibody without the masking moiety). In some embodiments, the activatable antibody is TY22404.

In some embodiments, an activatable antibody of the present disclosure comprises: (a) a masking moiety (MM); (b) a cleavable moiety (CM); and (c) a target binding moiety (TBM). In some embodiments, the MM is any of the masking moieties described herein. In some embodiments, the CM is any of the cleavable moieties described herein. In some embodiments, the TBM is any of the target binding moieties described herein (e.g., a target binding moiety (TBM) comprising an antibody light chain variable region and/or an antibody heavy chain variable region, such as a VH and/or VL of any of the anti-CTLA4 antibodies described herein).

In some embodiments, the activatable antibody comprises: (a) a polypeptide comprising, from N-terminus to C-terminus, a masking moiety (MM), a cleavable moiety (CM), and a target binding moiety (TBM), where the MM is any of the masking moieties described herein, the CM is any of the cleavable moieties described herein, and where the TBM comprises an antibody light chain variable region (VL); and (b) an antibody heavy chain variable region (VH).

In some embodiments, the activatable antibody comprises: (a) a polypeptide comprising, from N-terminus to C-terminus, a masking moiety (MM), a cleavable moiety (CM), and a target binding moiety (TBM), where the MM is any of the masking moieties described herein, the CM is any of the cleavable moieties described herein, and where the TBM comprises an antibody heavy chain variable region (VH); and (b) an antibody light chain variable region (VL).

In some embodiments, the activatable antibody comprises: a polypeptide comprising, from N-terminus to C-terminus, a masking moiety (MM), a cleavable moiety (CM), and a target binding moiety (TBM), where the MM is any of the masking moieties described herein, the CM is any of the cleavable moieties described herein, and where the TBM comprises an antibody heavy chain variable region (VH) and an antibody light chain variable region (VL).

The term “activatable binding polypeptide”, “ABP”, or “activatable antibody” includes a polypeptide that comprises a target binding moiety (TBM), a cleavable moiety (CM), and a masking moiety (MM). In some embodiments, the TBM comprises an amino acid sequence that binds to a target. In some embodiments, the TBM comprises an antigen binding domain (ABD) of an antibody or antibody fragment thereof (e.g., any of the antibodies or antigen binding fragments described herein). In some embodiments, the antigen binding domain comprises a heavy chain variable region comprising one, two, or three of the heavy chain variable region HVRs described herein, and a light chain variable region comprising one, two, or three of the light chain variable region HVRs described herein (e.g., one, two, or three of the heavy chain variable region HVR sequences, and/or one, two, or three of the light chain variable region HVR sequences as shown in Table A, including all six HVRs of any of the exemplary antibodies as shown in Table A). In some embodiments, the antigen binding domain comprises a heavy chain variable region comprising any of the heavy chain variable region sequences described herein, and a light chain variable region comprising any of the light chain variable region sequences described herein (e.g., a heavy chain variable region sequence and/or a light chain variable region sequence as shown in Table B). In some embodiments, the TBM (e.g., comprising an ABD) comprises an antibody light chain variable region (VL) and an antibody heavy chain variable region (VH), wherein the VH and VL forms a binding domain that binds to the target in the absence of the MM. In some embodiments, the VH and VL are covalently linked, e.g., in an scFv. In some embodiments, the VH and VL are not covalently linked. In some embodiments, the VH and VL form a Fab fragment. In some embodiments, the VH is linked to an antibody heavy chain constant region, and the VL is linked to an antibody light chain constant region.

TABLE A
anti-CTLA4 HVR sequences

| Ab | | | | | | |
|---|---|---|---|---|---|---|
| name: | HVR-H1 | HVR-H2 | HVR-H3 | HVR-L1 | HVR-L2 | HVR-L3 |
| TY21585 | FTFSDYAI | IGIISPSSGSTNY | ARDIHSGSSG | RASESVDF | DASNR | YCQHYTS |
| | HWV | AQKFQGRV | YYYGFDV | FGISFLA | ATGI | SPPVYT |
| | (SEQ ID | (SEQ ID NO: 30) | (SEQ ID NO: | (SEQ ID NO: | (SEQ | (SEQ ID |
| | NO: 18) | | 40) | 53) | ID NO: | NO: 70) |
| | | | | | 66) | |
| TY21586 | YSITSGY | VSSISGSGSTTY | ARDGFGYFD | SASSSVSY | DASSL | YCVQGLQ |
| | YWAWI | YADSVKGRF | Y | VY | ESGV | TPWT |
| | (SEQ ID | (SEQ ID NO: 31) | (SEQ ID NO: | (SEQ ID NO: | (SEQ | (SEQ ID |
| | NO: 19) | | 41) | 54) | ID NO: | NO: 71) |
| | | | | | 67) | |
| TY21587 | FTFSDYGI | IGEIYHSGSTYYS | ARDVAPGSS | RASQGIGSS | DASNR | YCQQYD |
| | HWV | PSLKSRV | GYYDGFDF | LA | ATGI | QWPPWT |
| | (SEQ ID | (SEQ ID NO: 32) | (SEQ ID NO: | (SEQ ID NO: | (SEQ | (SEQ ID |
| | NO: 20) | | 42) | 55) | ID NO: | NO: 72) |
| | | | | | 66) | |
| TY21588 | YSISSGY | VSGISGYGGSTY | ARHSYYGSG | RASESVDF | DASNL | YCQQSYS |
| | HWDWI | YADSVKGRF | NFDY | FGKSFLH | ETGV | WPPT |
| | (SEQ ID | (SEQ ID NO: 33) | (SEQ ID NO: | (SEQ ID NO: | (SEQ | (SEQ ID |
| | NO: 21) | | 43) | 56) | ID NO: | NO: 73) |
| | | | | | 68) | |
| TY21589 | FTFSDYW | IGWISPSGGGTK | ARGAYEFDY | RASQSVSS | DASNR | YCQQSYP |
| | IHWV | YAQKFQGRV | (SEQ ID NO: | RFLA | ATGI | TPLT |
| | (SEQ ID | (SEQ ID NO: 34) | 44) | (SEQ ID NO: | (SEQ | (SEQ ID |
| | NO: 22) | | | 57) | ID NO: | NO: 74) |
| | | | | | 66) | |
| TY21580 | YSISSGY | LARIDWDDDKY | ARSYVYFDY | RASQSVRG | DASNR | YCQQSSS |
| | HWSWI | YSTSLKSRL | (SEQ ID NO: | RFLA | ATGI | WPPT |
| | (SEQ ID | (SEQ ID NO: 35) | 45) | (SEQ ID NO: | (SEQ | (SEQ ID |
| | NO: 23) | | | 58) | ID NO: | NO: 75) |
| | | | | | 66) | |
| TY21591 | FSLSTGG | IGEIYHSGSTYYS | ARRIATATYF | RASQTVFS | DASNR | YCQQSYY |
| | VAVSWI | PSLKSRV | DY | RYLA | ATGI | WPPWT |
| | (SEQ ID | (SEQ ID NO: 32) | (SEQ ID NO: | (SEQ ID NO: | (SEQ | (SEQ ID |
| | NO: 24) | | 46) | 59) | ID NO: | NO: 76) |
| | | | | | 66) | |
| TY21686 | FSLSTGG | VSAISGYGSTTY | ARLPYSAYA | RASQGVSS | AASTL | YCQHHY |
| | VAVGWI | YADSVKGRF | FDY | YLA | QSGV | GTPLT |
| | (SEQ ID | (SEQ ID NO: 36) | (SEQ ID NO: | (SEQ ID NO: | (SEQ | (SEQ ID |
| | NO: 25) | | 47) | 60) | ID NO: | NO: 77) |
| | | | | | 69) | |
| TY21687 | FTFSGYAI | IGIISPSGGGTKY | ARHPFAY | RASQSVDF | DASNR | YCQQYVS |
| | HWV | AQKFQGRV | (SEQ ID NO: | YGISFLD | ATGI | SPPEYT |
| | (SEQ ID | (SEQ ID NO: 37) | 48) | (SEQ ID NO: | (SEQ | (SEQ ID |
| | NO: 26) | | | 61) | ID NO: | NO: 78) |
| | | | | | 66) | |
| TY21689 | YTFSGYG | IGEIYHSGSTYYS | ARRIDAFDI | RASQSVDF | DASSL | YCQQRDS |
| | IHWV | PSLKSRV | (SEQ ID NO: | DGFSFLH | ESGV | WPYT |
| | (SEQ ID | (SEQ ID NO: 32) | 49) | (SEQ ID NO: | (SEQ | (SEQ ID |
| | NO: 27) | | | 62) | ID NO: | NO: 79) |
| | | | | | 67) | |
| TY21680 | YTFSGYA | IGIISPSGGGTKY | ARLYDVAY | RASQSVDF | DASSL | YCEQSLE |
| | IHWV | AQKFQGRV | (SEQ ID NO: | HGKSFLH | ESGV | VPFT |
| | (SEQ ID | (SEQ ID NO: 37) | 50) | (SEQ ID NO: | (SEQ | (SEQ ID |
| | NO: 28) | | | 63) | ID NO: | NO: 80) |
| | | | | | 67) | |
| TY21691 | FTFSDYAI | IGIISPSGGSTKY | ARLGYGYFD | RASQSVDF | DASSL | YCVQALQ |
| | HWV | AQKFQGRV | V | YGISFLH | ESGV | LPLT |
| | (SEQ ID | (SEQ ID NO: 38) | (SEQ ID NO: | (SEQ ID NO: | (SEQ | (SEQ ID |
| | NO: 18) | | 51) | 64) | ID NO: | NO: 81) |
| | | | | | 67) | |
| TY21692 | YSITSGH | IGDISHSGSTYYS | ARGSRTGYF | RASQSISSY | DASNL | YCQHHY |
| | YWSWI | QSLKSRV | DY | LN | ETGV | GTPLT |
| | (SEQ ID | (SEQ ID NO: 39) | (SEQ ID NO: | (SEQ ID NO: | (SEQ | (SEQ ID |
| | NO: 29) | | 52) | 65) | ID NO: | NO: 77) |
| | | | | | 68) | |

TABLE B
anti-CTLA4 variable region amino acid sequences

| Ab | | |
|---|---|---|
| name: | VH: | VL: |
| TY21585 | EVQLVESGGGLVQPGGSLRLSCAASGFTF | DIQLTQSPSSLSASVGDRVTITCRASESV |
| | SDYAIHWVRQAPGKGLEWIGIISPSSGST | DFFGISFLAWYQQKPGKAPKLLIYDAS |
| | NYAQKFQGRVTISRDNSKNTLYLQLNSL | NRATGIPSRFSGSGSGTDFTLTISSLQPE |
| | RAEDTAVYYCARDIHSGSSGYYYGFDV | DFATYYCQHYTSSPPVYTFGQGTKVEI |
| | WGQGTLVTVSS | KR |
| | (SEQ ID NO: 82) | (SEQ ID NO: 95) |
| TY21586 | EVQLVESGGGLVQPGGSLRLSCAASGYSI | DIQLTQSPSSLSASVGDRVTITCSASSSV |
| | TSGYYWAWIRQAPGKGLEWVSSISGSGS | SYVYWYQQKPGKAPKLLIYDASSLESG |
| | TTYYADSVKGRFTISRDNSKNTLYLQLNS | VPSRFSGSGSGTDFTLTISSLQPEDFATY |
| | LRAEDTAVYYCARDGFGYFDYWGQGTL | YCVQGLQTPWTFGQGTKVEIKR |
| | VTVSS | (SEQ ID NO: 96) |
| | (SEQ ID NO: 83) | |
| TY21587 | EVQLVESGGGLVQPGGSLRLSCAASGFTF | DIQLTQSPSSLSASVGDRVTITCRASQGI |
| | SDYGIHWVRQAPGKGLEWIGEIYHSGST | GSSLAWYQQKPGKAPKLLIYDASNRAT |
| | YYSPSLKSRVTISRDNSKNTLYLQLNSLR | GIPSRFSGSGSGTDFTLTISSLQPEDFAT |
| | AEDTAVYYCARDVAPGSSGYYDGFDFW | YYCQQYDQWPPWTFGQGTKVEIKR |
| | GQGTLVTVSS | (SEQ ID NO: 97) |
| | (SEQ ID NO: 84) | |
| TY21588 | EVQLVESGGGLVQPGGSLRLSCAASGYSI | DIQLTQSPSSLSASVGDRVTITCRASESV |
| | SSGYHWDWIRQAPGKGLEWVSGISGYG | DFFGKSFLHWYQQKPGKAPKLLIYDAS |
| | GSTYYADSVKGRFTISRDNSKNTLYLQLN | NLETGVPSRFSGSGSGTDFTLTISSLQPE |
| | SLRAEDTAVYYCARHSYYGSGNFDYWG | DFATYYCQQSYSWPPTFGQGTKVEIKR |
| | QGTLVTVSS | (SEQ ID NO: 98) |
| | (SEQ ID NO: 85) | |
| TY21589 | EVQLVESGGGLVQPGGSLRLSCAASGFTF | DIQLTQSPSSLSASVGDRVTITCRASQS |
| | SDYWIHWVRQAPGKGLEWIGWISPSGG | VSSRFLAWYQQKPGKAPKLLIYDASNR |
| | GTKYAQKFQGRVTISRDNSKNTLYLQLN | ATGIPSRFSGSGSGTDFTLTISSLQPEDF |
| | SLRAEDTAVYYCARGAYEFDYWGQGTL | ATYYCQQSYPTPLTFGQGTKVEIKR |
| | VTVSS | (SEQ ID NO: 99) |
| | (SEQ ID NO: 86) | |
| TY21580 | EVQLVESGGGLVQPGGSLRLSCAASGYSI | DIQLTQSPSSLSASVGDRVTITCRASQS |
| | SSGYHWSWIRQAPGKGLEWLARIDWDD | VRGRFLAWYQQKPGKAPKLLIYDASN |
| | DKYYSTSLKSRLTISRDNSKNTLYLQLNS | RATGIPSRFSGSGSGTDFTLTISSLQPED |
| | LRAEDTAVYYCARSYVYFDYWGQGTLV | FATYYCQQSSSWPPTFGQGTKVEIKR |
| | TVSS | (SEQ ID NO: 100) |
| | (SEQ ID NO: 87) | |
| TY21591 | EVQLVESGGGLVQPGGSLRLSCAASGFSL | DIQLTQSPSSLSASVGDRVTITCRASQT |
| | STGGVAVSWIRQAPGKGLEWIGEIYHSGS | VFSRYLAWYQQKPGKAPKLLIYDASN |
| | TYYSPSLKSRVTISRDNSKNTLYLQLNSL | RATGIPSRFSGSGSGTDFTLTISSLQPED |
| | RAEDTAVYYCARRIATATYFDYWGQGTL | FATYYCQQSYYWPPWTFGQGTKVEIK |
| | VTVSS | R |
| | (SEQ ID NO: 88) | (SEQ ID NO: 101) |
| TY21686 | EVQLVESGGGLVQPGGSLRLSCAASGFSL | DIQLTQSPSSLSASVGDRVTITCRASQG |
| | STGGVAVGWIRQAPGKGLEWVSAISGYG | VSSYLAWYQQKPGKAPKLLIYAASTLQ |
| | STTYYADSVKGRFTISRDNSKNTLYLQLN | SGVPSRFSGSGSGTDFTLTISSLQPEDFA |
| | SLRAEDTAVYYCARLPYSAYAFDYWGQG | TYYCQHHYGTPLTFGQGTKVEIKR |
| | TLVTVSS | (SEQ ID NO: 102) |
| | (SEQ ID NO: 89) | |
| TY21687 | EVQLVESGGGLVQPGGSLRLSCAASGFTF | DIQLTQSPSSLSASVGDRVTITCRASQS |
| | SGYAIHWVRQAPGKGLEWIGIISPSGGGT | VDFYGISFLDWYQQKPGKAPKLLIYDA |
| | KYAQKFQGRVTISRDNSKNTLYLQLNSL | SNRATGIPSRFSGSGSGTDFTLTISSLQP |
| | RAEDTAVYYCARHPFAYWGQGTLVTVSS | EDFATYYCQQYVSSPPEYTFGQGTKVE |
| | (SEQ ID NO: 90) | IKR |
| | | (SEQ ID NO: 103) |
| TY21689 | EVQLVESGGGLVQPGGSLRLSCAASGYT | DIQLTQSPSSLSASVGDRVTITCRASQS |
| | FSGYGIHWVRQAPGKGLEWIGEIYHSGS | VDFDGFSFLHWYQQKPGKAPKLLIYD |
| | TYYSPSLKSRVTISRDNSKNTLYLQLNSL | ASSLESGVPSRFSGSGSGTDFTLTISSLQ |
| | RAEDTAVYYCARRIDAFDIWGQGTLVTV | PEDFATYYCQQRDSWPYTFGQGTKVEI |
| | SS | KR |
| | (SEQ ID NO: 91) | (SEQ ID NO: 104) |
| TY21680 | EVQLVESGGGLVQPGGSLRLSCAASGYT | DIQLTQSPSSLSASVGDRVTITCRASQS |
| | FSGYAIHWVRQAPGKGLEWIGIISPSGGG | VDFHGKSFLHWYQQKPGKAPKLLIYD |
| | TKYAQKFQGRVTISRDNSKNTLYLQLNSL | ASSLESGVPSRFSGSGSGTDFTLTISSLQ |
| | RAEDTAVYYCARLYDVAYWGQGTLVTV | PEDFATYYCEQSLEVPFTFGQGTKVEIK |
| | SS | R |
| | (SEQ ID NO: 92) | (SEQ ID NO: 105) |
| TY21691 | EVQLVESGGGLVQPGGSLRLSCAASGFTF | DIQLTQSPSSLSASVGDRVTITCRASQS |
| | SDYAIHWVRQAPGKGLEWIGIISPSGGST | VDFYGISFLHWYQQKPGKAPKLLIYDA |
| | KYAQKFQGRVTISRDNSKNTLYLQLNSL | SSLESGVPSRFSGSGSGTDFTLTISSLQP |
| | RAEDTAVYYCARLGYGYFDVWGQGTLV | EDFATYYCVQALQLPLTFGQGTKVEIK |
| | TVSS | R |
| | (SEQ ID NO: 93) | (SEQ ID NO: 106) |
| TY21692 | EVQLVESGGGLVQPGGSLRLSCAASGYSI | DIQLTQSPSSLSASVGDRVTITCRASQSI |
| | TSGHYWSWIRQAPGKGLEWIGDISHSGS | SSYLNWYQQKPGKAPKLLIYDASNLET |
| | TYYSQSLKSRVTISRDNSKNTLYLQLNSL | GVPSRFSGSGSGTDFTLTISSLQPEDFAT |
| | RAEDTAVYYCARGSRTGYFDYWGQGTL | YYCQHHYGTPLTFGQGTKVEIKR |
| | VTVSS | (SEQ ID NO: 107) |
| | (SEQ ID NO: 94) | |

In some embodiments, the activatable antibody comprises a polypeptide comprising the structure, from N-terminus to C-terminus, of: masking moiety (MM)-cleavable moiety (CM)-VL, and the activatable antibody further comprises a second polypeptide comprising a VH (e.g., a Fab fragment). In some embodiments, the activatable antibody comprises a polypeptide comprising the structure, from N-terminus to C-terminus, of: masking moiety (MM)-cleavable moiety (CM)-VL-VH (e.g., an scFv). In some embodiments, the activatable antibody comprises a polypeptide comprising the structure, from N-terminus to C-terminus, of: masking moiety (MM)-cleavable moiety (CM)-VH, and the activatable antibody further comprises a second polypeptide comprising a VL (e.g., a Fab fragment). In some embodiments, the activatable antibody comprises a polypeptide comprising the structure, from N-terminus to C-terminus, of: masking moiety (MM)-cleavable moiety (CM)-VH-VL (e.g., an scFv).

The CM generally includes an amino acid sequence that is cleavable, for example, serves as the substrate for an enzyme and/or a cysteine-cysteine pair capable of forming a reducible disulfide bond. As such, when the terms “cleavage,” “cleavable,” “cleaved” and the like are used in connection with a CM, the terms encompass enzymatic cleavage, e.g., by a protease, as well as disruption of a disulfide bond between a cysteine-cysteine pair via reduction of the disulfide bond that can result from exposure to a reducing agent.

The MM refers to an amino acid sequence that, when the CM of the activatable antibody is intact (e.g., uncleaved by a corresponding enzyme, and/or containing an unreduced cysteine-cysteine disulfide bond), the MM interferes with or inhibits binding of the TBM to its target. In some embodiments, the MM interferes with or inhibits binding of the TBM to its target so efficiently that binding of the TBM to its target is extremely low and/or below the limit of detection (e.g., binding cannot be detected in an ELISA or flow cytometry assay). The amino acid sequence of the CM may overlap with or be included within the MM. It should be noted that for sake of convenience “ABP” or “activatable antibody” are used herein to refer to an ABP or activatable antibody in both their uncleaved (or “native”) state, as well as in their cleaved state. It will be apparent to the ordinarily skilled artisan that in some embodiments a cleaved ABP may lack an MM due to cleavage of the CM, e.g., by a protease, resulting in release of at least the MM (e.g., where the MM is not joined to the ABP by a covalent bond (e.g., a disulfide bond between cysteine residues)). Exemplary ABPs are described in more detail below.

In some embodiments, the masking moiety (MM) interferes with, obstructs, reduces the ability of, prevents, inhibits, or competes with the target binding moiety for binding to its target (e.g., an “inactive activatable antibody). In some embodiments, the masking moiety (MM) interferes with, obstructs, reduces, prevents, inhibits, or competes with the target binding moiety for binding to its target only when the polypeptide has not been activated (e.g., activated by a change in pH (increased or decreased), activated by a temperature shift (increased or decreased), activated after being contacted with a second molecule (such as a small molecule or a protein ligand), etc.). In some embodiments, activation induces cleavage of the polypeptide within the cleavage moiety. In some embodiments, activation induces conformation changes in the polypeptide (e.g., displacement of the masking moiety (MM)), leading to the masking moiety no longer preventing the activatable antibody from binding to its target. In some embodiments, the masking moiety (MM) interferes with, obstructs, reduces the ability of, prevents, inhibits, or competes with the target binding moiety for binding to its target only when the cleavable moiety (CM) has not been cleaved by one or more proteases that cleave within the cleavable moiety (CM). In some embodiments, the masking moiety (MM) has a masking efficiency of at least about 2.0 (e.g., at least about 2.0, at least about 3.0, at least about 4.0, at least about 5.0, at least about 6.0, at least about 7.0, at least about 8.0, at least about 9.0, at least about 10, at least about 25, at least about 50, at least about 75, at least about 100, at least about 150, at least about 200, at least about 300, at least about 400, at least about 500, etc.) prior to activation. In some embodiments, masking efficiency is measured as the difference in affinity of an activatable antibody comprising the masking moiety (MM) for binding its target (before activation) relative to the affinity of a polypeptide lacking the masking moiety for binding its target (e.g., the difference in affinity for a target antigen (such as CTLA4) of an activatable antibody comprising a masking moiety (MM) (before activation) relative to a parental antibody lacking the masking moiety (MM), or the difference in affinity for a target antigen (such as CTLA4) of an activatable antibody comprising a masking moiety (MM) (before activation) relative to the affinity for the target antigen of the activatable antibody after activation). In some embodiments, the masking efficiency is measured by dividing the EC₅₀ for binding of an activatable antibody comprising a masking moiety (MM) (before activation) by the EC₅₀ of the parental antibody (e.g., by measuring EC₅₀ by ELISA). In some embodiments, masking efficiency is measured as the difference in affinity of an activatable antibody comprising the masking moiety (MM) for binding its target before activation relative to the affinity of the activatable antibody comprising the masking moiety (MM) for binding its target after activation (e.g., the difference in affinity for a target antigen (such as CTLA4) of an activatable antibody before activation relative to the activatable antibody after activation). In some embodiments, the masking moiety (MM) binds to the target binding moiety (TBM), and prevents the activatable antibody from binding to its target (e.g., an “inactive” activatable antibody). In some embodiments, the masking moiety (MM) has a dissociation constant for binding to the target binding moiety (TBM) that is greater than the dissociation constant of the target binding moiety (TBM) for its target.

In some embodiments, the masking moiety (MM) does not interfere with, obstruct, reduce the ability of, prevent, inhibit, or compete with the target binding moiety (TBM) for binding to its target after the activatable antibody has been activated (e.g., activated by treatment with one or more proteases that cleave within the cleavable moiety (CM), activated by a change in pH (increased or decreased), activated by a temperature shift (increased or decreased), activated after being contacted with a second molecule (such as an enzyme or a protein ligand), etc.). In some embodiments, the masking moiety (MM) does not interfere with, obstruct, reduce the ability of, prevent, inhibit, or compete with the target binding moiety (TBM) for binding its target after the cleavable moiety (CM) has been cleaved by one or more proteases that cleave within the cleavable moiety (CM). In some embodiments, the masking moiety (MM) has a masking efficiency of at most about 1.75 (e.g., at most about 1.75, at most about 1.5, at most about 1.4, at most about 1.3, at most about 1.2, at most about 1.1, at most about 1.0, at most about 0.9, at most about 0.8, at most about 0.7, at most about 0.6, or at most about 0.5, etc.) after activation (e.g., the relative affinity of the activatable antibody after activation as compared to the affinity of a parental antibody).

In some embodiments, an activatable antibody of the present disclosure: contains a masking moiety (MM) comprising a pair of cysteine residues at fixed positions to ensure that the activatable antibodies have constrained conformations, and/or harbor few or no chemically labile residues (such as methionine or tryptophan). Advantageously, the inclusion of a pair of cysteine residues at fixed positions ensured that the activatable antibodies had constrained conformations, tending to exhibit increased binding affinity and/or specificity. Furthermore, activatable antibodies of the present disclosure included masking moieties with few to no unfavorable residues for manufacturing processes, such as methionine or tryptophan.

In some embodiments, activatable antibodies of the present disclosure are context-dependent (e.g., are activated (are only capable of binding their targets) in certain contexts (such as in the protease-rich tumor microenvironment)). In some embodiments, the activatable antibodies of the present disclosure provide improved safety over more traditional, non-activatable antibodies (e.g., show reduced toxicity, do not induce significant alterations to the weights of many organs, do not alter liver histopathology, hematology, and/or blood biochemistry, etc.). In some embodiments, activatable antibodies of the present disclosure have improved pharmacokinetic properties as compared to more traditional, non-activatable antibodies (e.g., have longer in vivo half-lives).

### Anti-CTLA4 Activatable Antibody Activities

In some embodiments, the present disclosure relates to activatable antibodies that bind to human CTLA4 when in active form (e.g., the activatable antibodies are active after cleavage in the cleavable moiety (e.g., with one or more proteases), but inactive prior to cleavage in the cleavable moiety (e.g., with one or more proteases)). In some embodiments, the activatable antibodies when in active form have at least one (e.g., at least one, at least two, at least three, at least four, at least five, at least six, at least seven, at least eight, or all nine) of the following functional properties: (a) bind to human, cynomolgus monkey, mouse, rat, and/or dog CTLA4 with a K_{D} of 500 nM or less, e.g., about 10 nM or less; (b) have antagonist activity on human CTLA4; (c) do not bind to human PD-1, PD-L1, PD-L2, LAG3, TIM3, B7-H3, CD95, CD120a, OX40, CD40, BTLA, VISTA, ICOS, and/or B7-H4 at concentration up to 100 nM; (d) are cross-reactive with monkey, mouse, rat, and/or dog CTLA4; (e) induces ADCC effects (e.g., on Tregs); (f) activates human PBMCs (e.g., stimulates secretion of IL-2 and/or IFNγ); (g) are capable of inhibiting tumor cell growth; (h) have therapeutic effect on a cancer; and (i) inhibit binding of human CTLA4 to human CD80 and/or human CD86. Also provided herein are one or more activatable antibodies that compete or cross-compete for binding to human CTLA4 with one or more of the CTLA4-targeting activatable antibodies and/or anti-CTLA4 antibodies described herein.

In some embodiments, the activatable antibodies bind to human, cynomolgus monkey, mouse, rat, and/or dog CTLA4 with a K_{D} of about 500 nM or more when in inactive form. In some embodiments, the activatable antibodies bind to human, cynomolgus monkey, mouse, rat, and/or dog CTLA4 with a K_{D} of about 500 nM or less when in active form (e.g., about 500 nM or less, about 450 nM or less, about 400 nM or less, about 350 nM or less, about 300 nM or less, about 250 nM or less, about 200 nM or less, about 150 nM or less, about 100 nM or less, about 90 nM or less, about 80 nM or less, about 70 nM or less, about 60 nM or less, about 50 nM or less, about 40 nM or less, about 30 nM or less, about 25 nM or less, about 20 nM or less, about 10 nM or less, about 1 nM or less, about 0.1 nM or less, etc.) In some embodiments, the activatable antibodies bind to human, cynomolgus monkey, mouse, rat, and/or dog CTLA4 with a K_{D} of about 350 nM or less when in active form. In some embodiments, the activatable antibodies bind to human CTLA4 with a K_{D} of about 100 nM or less when in active form. In some embodiments, the activatable antibodies bind to human CTLA4 with a K_{D} of about 50 nM or less when in active form. In some embodiments, the activatable antibodies bind to human CTLA4 with a K_{D} of about 10 nM or less when in active form. Methods of measuring the K_{D} of an activatable antibody may be carried out using any method known in the art, including for example, by surface plasmon resonance, an ELISA, isothermal titration calorimetry, a filter binding assay, an EMSA, etc. In some embodiments, the K_{D} is measured by an ELISA (see e.g., the Examples below).

In some embodiments, the activatable antibodies do not have antagonist activity on human CTLA4 when in inactive form. In some embodiments, the activatable antibodies have antagonist activity on human CTLA4 when in active form (e.g., induces ADCC effects (such as against Tregs), activates PBMCs (such as by activating, inducing, and/or stimulating IL-2 and/or IFNγ secretion), bocks binding of human CTLA4 to human CD80 and/or human CD86, etc.). In some embodiments, the activatable antibodies repress one or more activities of human CTLA4 when in active form (e.g., repress one or more activities of human CTLA4 when a cell (such as a human cell) expressing human CTLA4 is contacted by an activatable antibody).

In some embodiments, when in inactive form, the activatable antibodies are not cross-reactive with monkey (e.g., cynomolgus monkey), mouse, rat, and/or dog CTLA4. In some embodiments, when in active form, the activatable antibodies are cross-reactive with monkey (e.g., cynomolgus monkey), mouse, rat, and/or dog CTLA4. In some embodiments, when in active form, the activatable antibodies are cross-reactive with monkey CTLA4. In some embodiments, when in active form, the activatable antibodies are cross-reactive with mouse CTLA4. In some embodiments, when in active form, the activatable antibodies are cross-reactive with rat CTLA4. In some embodiments, when in active form, the activatable antibodies are cross-reactive with dog CTLA4. In some embodiments, when in active form, the activatable antibodies are cross reactive with monkey and mouse CTLA4; monkey and rat CTLA4; monkey and dog CTLA4; mouse and rat CTLA4; mouse and dog CTLA4; rat and dog CTLA4; monkey, mouse, and rat CTLA4; monkey, mouse, and dog CTLA4; monkey, rat, and dog CTLA4; mouse, rat, and dog CTLA4; or monkey, mouse, rat, and dog CTLA4. In some embodiments, when in active form, the activatable binding polypeptides are cross-reactive at about 350 nM (e.g., at about 1 nM, at about 10 nM, at about 25 nM, at about 50 nM, at about 75 nM, at about 100 nM, at about 150 nM, at about 200 nM, at about 250 nM, at about 300 nM, at about 350 nM). Methods of measuring cross-reactivity are known in the art, including, without limitation, surface plasmon resonance, an ELISA, isothermal titration calorimetry, a filter binding assay, an EMSA, etc.

In some embodiments, the activatable antibodies do not induce ADCC effects (e.g., against CTLA4-expressing human cells such as Tregs) when in inactive form. In some embodiments, the activatable antibodies have reduced ADCC effects (e.g., against CTLA4-expressing human cells such as Tregs) when in inactive form as compared to a control binding polypeptide (e.g., a parental antibody). In some embodiments, the activatable antibodies induce ADCC effects (e.g., against CTLA4-expressing such as Tregs) when in active form. Methods of measuring ADCC effects (e.g., in vitro methods) are known in the art, including, without limitation, via the methods described in the Examples below. In some embodiments, when in inactive form, the activatable antibodies induce ADCC effects by less than about 10% (e.g., induce ADCC by less than about 10%, less than about 5%, less than about 1%, etc.) relative to a control (e.g., a parental antibody). In some embodiments, when in active form, the activatable antibodies induce ADCC effects by more than about 10% (e.g., induce ADCC by more than about 10%, more than about 15%, more than about 20%, more than about 25%, more than about 30%, more than about 35%, more than about 40%, etc.) relative to a control (e.g., an isotype control).

In some embodiments, the activatable antibodies are capable of inhibiting tumor cell growth and/or proliferation. In some embodiments, the tumor cell growth and/or proliferation is inhibited by at least about 5% (e.g., at least about 5%, at least about 10%, at least about 20%, at least about 30%, at least about 40%, at least about 50%, at least about 60%, at least about 70%, at least about 80%, at least about 90%, or at least about 99%) when contacted with the activatable antibodies relative to corresponding tumor cells not contacted with the activatable antibodies (or relative to corresponding tumor cells contacted with an isotype control antibody). In some embodiments, the activatable antibodies are capable of reducing tumor volume in a subject when the subject is administered the activatable antibodies. In some embodiments, the activatable antibodies are capable of reducing tumor volume in a subject by at least about 5% (e.g., at least about 5%, at least about 10%, at least about 20%, at least about 30%, at least about 40%, at least about 50%, at least about 60%, at least about 70%, at least about 80%, at least about 90%, or at least about 99%) relative to the initial tumor volume in the subject (e.g., prior to administration of the activatable antibodies; as compared to a corresponding tumor in a subject administered an isotype control antibody). Methods of monitoring tumor cell growth/proliferation, tumor volume, and/or tumor inhibition are known in the art, including, for example, via the methods described in the Examples below.

In some embodiments, the activatable antibodies have therapeutic effect on a cancer. In some embodiments, the activatable antibodies reduce one or more signs or symptoms of a cancer. In some embodiments, a subject suffering from a cancer goes into partial or complete remission when administered the activatable antibodies.

In some embodiments, the present disclosure provides isolated activatable antibodies that, when in active form, compete or cross-compete for binding to human CTLA4 with an antibody comprising: a) an HVR-H1 comprising the amino acid sequence of SEQ ID NO: 23; an HVR-H2 comprising the amino acid sequence of SEQ ID NO: 35; and an HVR-H3 comprising the amino acid sequence of SEQ ID NO: 45; and/or b) an HVR-L1 comprising the amino acid sequence of SEQ ID NO: 58; an HVR-L2 comprising the amino acid sequence of SEQ ID NO: 66; and an HVR-L3 comprising the amino acid sequence of SEQ ID NO: 75. In some embodiments, the present disclosure provides isolated activatable antibodies that, when in active form, compete or cross-compete for binding to human CTLA4 with an antibody comprising: a) a heavy chain variable region comprising the amino acid sequence of SEQ ID NO: 87; and/or b) a light chain variable region comprising the amino acid sequence of SEQ ID NO: 100. The ability of an activatable antibody to compete or cross-compete for binding with an antibody can be determined using standard binding assays known in the art, such as BIAcore analysis, ELISA assays, or flow cytometry. For example, one can allow an antibody (e.g., as described above) to bind to human CTLA4 under saturating conditions and then measure the ability of the test activatable antibody (when in active form) to bind to the CTLA4. If the test activatable antibody is able to bind to the CTLA4 at the same time as the antibody, then the test activatable antibody binds to a different epitope then the antibody. However, if the test activatable antibody is not able to bind to the CTLA4 at the same time, then the test activatable antibody binds to the same epitope, an overlapping epitope, or an epitope that is in close proximity to the epitope bound by the antibody. This experiment can be performed using various methods, such as ELISA, RIA, FACS or surface plasmon resonance.

In some embodiments, the activatable antibodies (when in inactive form) do not inhibit the binding between CTLA4 and one or more of its binding partners (e.g., human CTLA4 and human CD80, human CTLA4 and human CD86). In some embodiments, the activatable antibodies (when in active form) inhibit the binding between CTLA4 and one or more of its binding partners (e.g., human CTLA4 and human CD80, human CTLA4 and human CD86). In some embodiments, the activatable antibodies inhibit the binding between CTLA4 and its ligand in vitro. In some embodiments, the activatable antibodies have a half maximal inhibitory concentration (IC₅₀) of about 500 nM or less (e.g., about 500 nM or less, about 400 nM or less, about 300 nM or less, about 200 nM or less, about 100 nM or less, about 50 nM or less, about 25 nM or less, about 10 nM or less, about 1 nM or less, etc.) for inhibiting binding of CTLA4 to CD80 and/or CD86. In some embodiments, the activatable antibodies have a half maximal inhibitory concentration (IC₅₀) of about 100 nM or less for inhibiting binding of CTLA4 to CD80 and/or CD86. In some embodiments, the activatable antibodies completely inhibit binding of human CTLA4 to CD80 and/or CD86 when provided at a concentration of about 100 nM or greater (e.g., about 100 nM or greater, about 500 nM or greater, about 1 μM or greater, about 10 μM or greater, etc.). As used herein, the term “complete inhibiting” or “completely inhibits” refers to the activatable antibody's ability to reduce binding between a first protein and a second protein by at least about 80% (e.g., at least about 80%, at least about 85%, at least about 90%, at least about 95%, at least about 99%, etc.). Methods of measuring the ability of an a polypeptide to inhibit binding of a first protein (e.g., human CTLA4) and a second protein (e.g., human CD80 or human CD86) are known in the art, including, without limitation, via BIAcore analysis, ELISA assays, and flow cytometry.

### Masking Moieties (MMs)

In some embodiments, the present disclosure relates to activatable antibodies comprising a masking moiety (MM). In some embodiments, the masking moiety (MM) comprises an amino acid sequence according to Formula (XIX): ZₘCZₙCZₒ(SEQ ID NO: 135), where m is from 2-10, n is from 3-10, and o is from 1-10, and each Z is independently an amino acid selected from the group consisting of D, A, Y, S, T, N, I, L, F, V, H, and P. In some embodiments, m is from 6-8. In some embodiments, m is 3, 4, 5, 6, 7, 8, 9, or 10. In some embodiments, m is 6. In some embodiments, n is from 6-8. In some embodiments, n is 3, 4, 5, 6, 7, 8, 9, or 10. In some embodiments, n is 6. In some embodiments, n is 8. In some embodiments, o is from 1-2. In some embodiments, o is 1, 2, 3, 4, 5, 6, 7, 8, 9, or 10. In some embodiments, o is 2.

In some embodiments, the masking moiety (MM) comprises an amino acid sequence according to Formula (XXI): Z₆CX₆CZ₂ (SEQ ID NO: 137), where each X is independently an amino acid selected from the group consisting of A, C, D, E, F, G, H, I, K, L, M, N, P, Q, R, S, T, V, W, and Y, and where each Z is independently an amino acid selected from the group consisting of D, A, Y, S, T, N, I, L, F, V, H, and P.

In some embodiments, the masking moiety (MM) comprises an amino acid sequence according to Formula (XXII): Z₆CX₈CZ₂ (SEQ ID NO: 138), where each X is independently an amino acid selected from the group consisting of A, C, D, E, F, G, H, I, K, L, M, N, P, Q, R, S, T, V, W, and Y, and where each Z is independently an amino acid selected from the group consisting of D, A, Y, S, T, N, I, L, F, V, H, and P.

In some embodiments, the first peptide (FP) comprises an amino acid sequence according to Formula (XXIII): (Z₆)C(Z₆)C(Z₂) (SEQ ID NO: 139), where each Z is independently an amino acid selected from the group consisting of D, A, Y, S, T, N, I, L, F, V, H, and P.

In some embodiments, the masking moiety (MM) comprises an amino acid sequence according to Formula (XXIV): (Z₆)C(Z₈)C(Z₂) (SEQ ID NO: 140), where each Z is independently an amino acid selected from the group consisting of D, A, Y, S, T, N, I, L, F, V, H, and P. In some embodiments, an activatable antibody comprises a masking moiety (MM) comprising a sequence selected from the group consisting of XₘCPDHPYPCXX (SEQ ID NO:181), XₘCDAFYPYCXX (SEQ ID NO:182), XₘCDSHYPYCXX (SEQ ID NO:183), and XₘCVPYYYACXX (SEQ ID NO:184), where m is from 2-10, and where each X is independently an amino acid selected from the group consisting of A, C, D, E, F, G, H, I, K, L, M, N, P, Q, R, S, T, V, W, and Y. In some embodiments, an activatable antibody comprises a masking moiety (MM) comprising the sequence EVGSYNFVADSCPDHPYPCSA (SEQ ID NO:189), EVGSYIVHHSDCDAFYPYCDS (SEQ ID NO:190), EVGSYYSAYPACDSHYPYCNS (SEQ ID NO:191), EVGSYPNPSSDCVPYYYACAY (SEQ ID NO:192), EVGSYYSAYPACDSHYPYCQS (SEQ ID NO:193), EVGSYPQPSSDCVPYYYACAY (SEQ ID NO:195), or EVGSYPNPASDCVPYYYACAY (SEQ ID NO:196). In some embodiments, the MM comprises the sequence of EDCVPYYYACAY (SEQ ID NO:213), EVGSSDCVPYYYACAY (SEQ ID NO:214), EDCDAFYPYCDS (SEQ ID NO:215), or EVGHSDCDAFYPYCDS (SEQ ID NO:216).

In some embodiments, the masking moiety (MM) comprises an amino acid sequence selected from NFVADSCPDHPYPCSA (SEQ ID NO: 141), IVHHSDCDAFYPYCDS (SEQ ID NO: 142), YSAYPACDSHYPYCNS (SEQ ID NO: 143), PNPSSDCVPYYYACAY (SEQ ID NO: 144), YSAYPACDSHYPYCQS (SEQ ID NO: 145), PQPSSDCVPYYYACAY (SEQ ID NO: 146), and PNPASDCVPYYYACAY (SEQ ID NO: 147).

In some embodiments, any of the masking moieties (MMs) described herein may further comprise one or more additional amino acid sequences (e.g., one or more polypeptide tags). Examples of suitable additional amino acid sequence may include, without limitation, purification tags (such as his-tags, flag-tags, maltose binding protein and glutathione-S-transferase tags), detection tags (such as tags that may be detected photometrically (e.g., red or green fluorescent protein, etc.)), tags that have a detectable enzymatic activity (e.g., alkaline phosphatase, etc.), tags containing secretory sequences, leader sequences, and/or stabilizing sequences, protease cleavage sites (e.g., furin cleavage sites, TEV cleavage sites, Thrombin cleavage sites), and the like. In some embodiments, the one or more additional amino acid sequences are at the N-terminus of the masking moiety (MM). In some embodiments, the additional amino acid sequence comprises or consists of the sequence EVGSY (SEQ ID NO: 148).

In some embodiments, the masking moiety binds to the target binding moiety (TBM) and inhibits the activatable antibody from binding to its target before activation (e.g., before treatment with one or more proteases that cleave within the cleavable moiety (CM), before undergoing a (local) change in pH (increased or decreased), before a temperature shift (increased or decreased), before being contacted with a second molecule (such as a small molecule or a protein ligand), etc.), but does not bind to the TBM and/or inhibit the activatable antibody from binding to its target after activation (e.g., after treatment with one or more proteases that cleave within the cleavable moiety (CM), after undergoing a (local) change in pH (increased or decreased), after a temperature shift (increased or decreased), after being contacted with a second molecule (such as a small molecule or a protein ligand), etc.). In some embodiments, the masking moiety (MM) inhibits binding of an activatable antibody to its target when the CM is not cleaved, but does not inhibit binding of the activatable antibody to its target when the CM is cleaved. In some embodiments, the masking moiety (MM) has a dissociation constant for binding to the TBM that is greater (e.g., at least about 1.5-fold greater, at least about 2-fold greater, at least about 2.5-fold greater, at least about 3-fold greater, at least about 3.5-fold greater, at least about 4-fold greater, at least about 4.5-fold greater, at least about 5-fold greater, at least about 10-fold greater, at least about 100-fold greater, at least about 500-fold greater, etc.) than the dissociation constant of the activatable antibody for its target (when in active form).

### Cleavable Moieties (CMs)

In some embodiments, the present disclosure relates to activatable antibodies comprising a cleavable moiety (CM). In some embodiments, the cleavable moiety (CM) is cleaved and/or disrupted by treatment with one or more proteases that cleave within the cleavable moiety (CM), by a change in pH (increased or decreased), by a temperature shift (increased or decreased), and/or by contact with a second molecule (such as a small molecule or a protein ligand), etc.)

In some embodiments, the cleavable moiety (CM) comprises at least a first cleavage site (CS₁) (e.g., a first protease cleavage site). In some embodiments, the first cleavage site is a first protease cleavage site. Any suitable protease cleavage site recognized and/or cleaved by any protease (e.g., a protease that is known to be co-localized with a target of an activatable antibody comprising the CM) known in the art may be used, including, for example, a protease cleavage site recognized and/or cleaved by urokinase-type plasminogen activator (uPA); matrix metalloproteinases (e.g., MMP-1, MMP-2, MMP-3, MMP-7, MMP-8, MMP-9, MMP-10, MMP-11, MMP-12, MMP-13, MMP-14, MMP-15, MMP-16, MMP-17, MMP-19, MMP-20, MMP-23, MMP-24, MMP-26, and/or MMP-27); Tobacco Etch Virus (TEV) protease; plasmin; Thrombin; PSA; PSMA; ADAMS/ADAMTS (e.g., ADAM8, ADAM9, ADAM10, ADAM12, ADAM15, ADAM17/TACE, ADAMDEC1, ADAMTS1, ADAMTS4, and/or ADAMTS5); caspases (e.g., Caspase-1, Caspase-2, Caspase-3, Caspase-4, Caspase-5, Caspase-6, Caspase-7, Caspase-8, Caspase-9, Caspase-10, Caspase-11, Caspase-12, Caspase-13, and/or Caspase-14); aspartate proteases (e.g., RACE and/or Renin); aspartic cathepsins (e.g., Cathepsin D and/or Cathepsin E); cysteine cathepsins (e.g., Cathepsin B, Cathepsin C, Cathepsin K, Cathepsin L, Cathepsin S, Cathepsin V/L2, and/or Cathepsin X/Z/P); cysteine proteinases (e.g., Cruzipain, Legumain, and/or Otubain-2); KLKs (e.g., KLK4, KLK5, KLK6, KLK7, KLK8, KLK10, KLK11, KLK13, and/or KLK14); metallo proteainases (e.g., Meprin, Neprilysin, PSMA, and/or BMP-1); serine proteases (e.g., activated protein C, Cathepsin A, Cathepsin G, Chymase, and/or coagulation factor proteases (such as FVIIa, FIXa, FXa, FXIa, FXIIa)); elastase; granzyme B; guanidinobenzoatase; HtrA1; human neutrophil elastase; lactoferrin; marapsin; NS3/4A; PACE4; tPA; tryptase; type II transmembrane serine proteases (TTSPs) (e.g., DESC1, DPP-4, FAP, Hepsin, Matriptase-2, MT-SP1/Matriptase, TMPRSS2, TMPRSS3 and/or TMPRSS4); etc. In some embodiments, the first protease cleavage site is a cleavage site for a protease selected from uPA, MMP-1, MMP-2, MMP-3, MMP-8, MMP-9, MMP-14, TEV protease, plasmin, Thrombin, Factor X, PSA, PSMA, Cathepsin D, Cathepsin K, Cathepsin S, ADAM10, ADAM12, ADAMTS, Caspase-1, Caspase-2, Caspase-3, Caspase-4, Caspase-5, Caspase-6, Caspase-7, Caspase-8, Caspase-9, Caspase-10, Caspase-11, Caspase-12, Caspase-13, Caspase-14, and TACE. In some embodiments, the first protease cleavage site is a cleavage site for a protease selected from uPA, MMP-2, and/or MMP-9. In some embodiments, the protease cleavage comprises an amino acid sequence selected from SGRSA (SEQ ID NO: 149), PLGLAG (SEQ ID NO: 150).

Any suitable linker (e.g., a flexible linker) known in the art may be used, including, for example: glycine polymers (G)n, where n is an integer of at least 1 (e.g., at least one, at least 2, at least 3, at least 4, at least 5, at least 6, at least 7, at least 8, at least 9, at least 10, etc.); glycine-serine polymers (GS)n, where n is an integer of at least 1 (e.g., at least one, at least 2, at least 3, at least 4, at least 5, at least 6, at least 7, at least 8, at least 9, at least 10, etc.) such as GGGGS (SEQ ID NO: 156), SGGS (SEQ ID NO: 157), GGSG (SEQ ID NO: 158), GGSGG (SEQ ID NO: 159), GSGSG (SEQ ID NO: 160), GSGGG (SEQ ID NO: 161), GGGSG (SEQ ID NO: 162), and/or GSSSG (SEQ ID NO: 163)); glycine-alanine polymers; alanine-serine polymers; and the like. Linker sequences may be of any length, such as from about 1 amino acid (e.g., glycine or serine) to about 20 amino acids (e.g., 20 amino acid glycine polymers or glycine-serine polymers), about 4 amino acids to about 10 amino acids, about 5 amino acids to about 9 amino acids, about 6 amino acids to about 8 amino acids, etc. In some embodiments, the linker is any of about 1, 2, 3, 4, 5, 6, 7, 8, 9, 10, 11, 12, 13, 14, 15, 16, 17, 18, 19, or 20 amino acids in length. In some embodiments, the linker comprises an amino acid sequence selected from SEQ ID NOS: 159-163. In some embodiments, the linker comprises an amino acid sequence of SEQ ID NO: 156 or 157.

In some embodiments, the cleavable moiety (CM) further comprises at least a second cleavage site (e.g., at least a second, at least a third, at least a fourth, at least a fifth, etc.). In some embodiments, the cleavable moiety (CM) further comprises a second cleavage site (CS₂). In some embodiments, the second cleavage site is a second protease cleavage site. The second protease cleavage site may be any suitable protease cleavage site recognized and/or cleaved by any of the proteases described above. In some embodiments, the first (CS₁) and second (CS₂) cleavage sites are protease cleavage sites recognized and/or cleaved by the same protease. In some embodiments, the first (CS₁) and second (CS₂) cleavage sites are protease cleavage sites recognized and/or cleaved by different proteases (e.g., the first protease cleavage site is recognized and/or cleaved by uPA, and the second protease cleavage site is recognized and/or cleaved by MMP-2; the first protease cleavage site is recognized and/or cleaved by uPA, and the second protease cleavage site is recognized and/or cleaved by MMP-9; In some embodiments, the at least second cleavage site (CS₂) is C-terminal to the first linker (L₁). In some embodiments, the cleavable moiety (CM) comprises a structure, from N-terminus to C-terminus, of: (CS₁)-L₁-(CS₂).

In some embodiments, the cleavable moiety (CM) further comprises at least a second linker (e.g., at least a second, at least a third, at least a fourth, at least a fifth, etc.). In some embodiments, the cleavable moiety (CM) further comprises a second linker (L₂). The second linker (L₂) may be any suitable linker described above. In some embodiments, the second linker comprises an amino acid sequence selected from SEQ ID NO: 156-163. In some embodiments, the first (L₁) and second (L₂) linkers are the same (e.g., both linkers comprise the sequence of SEQ ID NO: 156 or 157). In some embodiments, the first (L₁) and second (L₂) linkers are different (e.g., the first linker (L₁) comprises the amino acid sequence of SEQ ID NO: 156, and the second linker (L₂) comprises the amino acid sequence of SEQ ID NO: 157, etc.). In some embodiments, the at least second linker (L₂) is C-terminal to the second cleavage site (CS₂). In some embodiments, the cleavable moiety (CM) comprises a structure, from N-terminus to C-terminus, of: (CS₁)-L₁-(CS₂)-L₂.

### Exemplary MM-CM Sequences

In some embodiments, an activatable antibody of the present disclosure comprises the structure, from N-terminus to C-terminus, of: (FP)-(PCS₁)-L₁-(PCS₂)-L₂. In some embodiments, an activatable antibody of the present disclosure comprises the amino acid sequence of: EVGSYNFVADSCPDHPYPCSASGRSAGGGGSPLGLAGSGGS (SEQ ID NO: 168); EVGSYIVHHSDCDAFYPYCDSSGRSAGGGGSPLGLAGSGGS (SEQ ID NO: 170); EVGSYYSAYPACDSHYPYCNSSGRSAGGGGSPLGLAGSGGS (SEQ ID NO: 172); EVGSYPNPSSDCVPYYYACAYSGRSAGGGGSPLGLAGSGGS (SEQ ID NO: 174); EVGSYYSAYPACDSHYPYCQSSGRSAGGGGSPLGLAGSGGS (SEQ ID NO: 176); EVGSYYSAYPACDSHYPYCNSAGRSAGGGGSPLGLAGSGGS (SEQ ID NO: 177); EVGSYPQPSSDCVPYYYACAYSGRSAGGGGSPLGLAGSGGS (SEQ ID NO: 178); and/or EVGSYPNPASDCVPYYYACAYSGRSAGGGGSPLGLAGSGGS (SEQ ID NO: 179). In some embodiments, a polypeptide of the present disclosure comprises the structure, from N-terminus to C-terminus, of: (FP)-(PCS₁)-L₁-(PCS₂)-L₂-(TBM).

In some embodiments, an activatable antibody comprises an amino acid sequence SGRSAGGGGTENLYFQGSGGS (SEQ ID NO:220), SGRSAGGGGTPLGLAGSGGS (SEQ ID NO:221), or SGRSAPLGLA (SEQ ID NO:222). In some embodiments, an activatable antibody comprises the sequence of EV(Zn)C(X₈)C(Z₂)SGRSA (SEQ ID NO:217), EDC(Z₆)C(Z₂)SGRSA (SEQ ID NO:218), or EDC(Z₆)C(Z₂)PLGLA (SEQ ID NO:219), where each X is independently an amino acid selected from the group consisting of A, C, D, E, F, G, H, I, K, L, M, N, P, Q, R, S, T, V, W, and Y, wherein n is 1-11 and wherein each Z is independently an amino acid selected from the group consisting of D, A, Y, S, T, N, I, L, F, V, H, and P.

In particular embodiments, the MM comprises an amino acid sequence EVGSYPNPSSDCVPYYYACAY (SEQ ID NO:192), and the cleavable moiety comprises an amino acid sequence SGRSAGGGGTPLGLAGSGGS (SEQ ID NO:221). In such embodiments, the MM and CM, from N-terminus to C-terminus comprises an amino acid sequence EVGSYPNPSSDCVPYYYACAY SGRSAGGGGTPLGLAGSGGS (SEQ ID NO:200). In some embodiments, the MM and the CM are covalently attached to the N-terminus of the light chain of the anti-CTLA4 antibody. In some embodiments, the MM and CM, from N-terminus to C-terminus comprises an amino acid sequence that has at least 90% or at least 95% sequence identity to SEQ ID NO:200.

### Target Binding Moieties (TBMs)

In some embodiments, the present disclosure relates to activatable antibodies comprising a target binding moiety (TBM). In some embodiments, the target binding moiety (TBM) comprises an antibody light chain variable region and/or an antibody heavy chain variable region. In some embodiments, the target binding moiety (TBM) comprises an antibody light chain variable region. In some embodiments, the target binding moiety (TBM) comprises an antibody heavy chain variable region. In some embodiments, the target binding moiety (TBM) comprises an antibody light chain variable region and an antibody heavy chain variable region.

In some embodiments, the target binding moiety (TBM) comprises a full length antibody light chain and/or a full length antibody heavy chain. The antibody light chain may be a kappa or lambda light chain. The antibody heavy chain may be in any class, such as IgG, IgM, IgE, IgA, or IgD. In some embodiments, the antibody heavy chain is in the IgG class, such as IgG1, IgG2, IgG3, or IgG4 subclass. An antibody heavy chain described herein may be converted from one class or subclass to another class or subclass using methods known in the art.

Any one or more of the target binding moieties (TBMs) described herein may incorporate: any of the HVR sequences described herein (e.g., one, two, or three of the heavy chain variable region HVR sequences, and/or one, two, or three of the light chain variable region HVR sequences as shown in Table A above); any of the heavy chain variable region sequences and/or light chain variable region sequences described herein (e.g., a heavy chain variable region sequence and/or a light chain variable region sequence as shown in Table B above); and/or any of any of the antibodies described herein.

In some embodiments, the target binding moiety (TBM) comprises a sequence of one or more of the anti-CTLA4 antibodies described herein, including antibodies described with reference to specific amino acid sequences of HVRs, variable regions (VL, VH), and/or light and heavy chains (e.g., IgG1, IgG2, IgG4). In some embodiments, the target binding moiety (TBM) comprises an antibody light chain variable region comprising an HVR-L1 comprising the amino acid sequence RASQSVRGRFLA (SEQ ID NO: 58), an HVR-L2 comprising the amino acid sequence DASNRATGI (SEQ ID NO: 66), and/or an HVR-L3 comprising the amino acid sequence YCQQSSSWPPT (SEQ ID NO: 75). In some embodiments, the target binding moiety (TBM) comprises an antibody light chain variable region comprising the amino acid sequence of SEQ ID NO: 100 or a sequence having at least 90% (e.g., 95%, 96%, 97%, 98% or 99%) sequence identity to the sequence of SEQ ID NO:100. In some embodiments, the target binding moiety (TBM) comprises an antibody heavy chain variable region comprising an HVR-H1 comprising the amino acid sequence YSISSGYHWSWI (SEQ ID NO: 23), an HVR-H2 comprising the amino acid sequence LARIDWDDDKYYSTSLKSRL (SEQ ID NO: 35), and/or an HVR-H3 comprising the amino acid sequence ARSYVYFDY (SEQ ID NO: 45). In some embodiments, the target binding moiety (TBM) comprises an antibody heavy chain variable region comprising the amino acid sequence of SEQ ID NO: 87 or a sequence having at least 90% (e.g., 95%, 96%, 97%, 98% or 99%) sequence identity to the sequence of SEQ ID NO:87. In some embodiments, the target binding moiety (TBM) comprises: a) an antibody light chain variable region comprising an HVR-L1 comprising the amino acid sequence RASQSVRGRFLA (SEQ ID NO: 58), an HVR-L2 comprising the amino acid sequence DASNRATGI (SEQ ID NO: 66), and/or an HVR-L3 comprising the amino acid sequence YCQQSSSWPPT (SEQ ID NO: 75); and b) an antibody heavy chain variable region comprising an HVR-H1 comprising the amino acid sequence YSISSGYHWSWI (SEQ ID NO: 23), an HVR-H2 comprising the amino acid sequence LARIDWDDDKYYSTSLKSRL (SEQ ID NO: 35), and/or an HVR-H3 comprising the amino acid sequence ARSYVYFDY (SEQ ID NO: 45). In some embodiments, the target binding moiety (TBM) comprises an antibody light chain variable region comprising the amino acid sequence of SEQ ID NO: 100, and an antibody heavy chain variable region comprising the amino acid sequence of SEQ ID NO: 87.

### Activatable Binding Polypeptide Properties

In some embodiments, an activatable binding polypeptide (i.e., activatable antibody) of the present disclosure comprises: (a) a masking moiety (MM), (b) a cleavable moiety, and (c) a target binding moiety. In some embodiments, the masking moiety (MM) binds to the target binding moiety (TBM) of the activatable antibody and reduces or inhibits binding of the activatable binding moiety to CTLA4 (e.g., human CTLA4), as compared to the binding of a corresponding binding polypeptide lacking the masking moiety to CTLA4 (e.g., human CTLA4) and/or as compared to the binding of a parental antibody to CTLA4 (e.g., human CTLA4).

In some embodiments, an “activatable” binding polypeptides refers to a binding polypeptide that exhibits a first level of binding to CTLA4 when in an inhibited, masked, and/or uncleaved state, and exhibits a second level of binding to CTLA4 in an uninhibited, unmasked, and/or cleaved state, where the second level of CTLA4 binding is greater than the first level of CTLA4 binding. In some embodiments, access to CTLA4 by the activatable binding polypeptide is greater after cleavage within the cleavable moiety (e.g., by one or more proteases).

In some embodiments, the activatable anti-CTLA4 antibody comprises a heavy chain comprising the amino acid sequence of SEQ ID NO:320 and a light chain comprising the amino acid sequence of SEQ ID NO:322. In some embodiments, the activatable anti-CTLA4 antibody comprises a heavy chain comprising the amino acid sequence of SEQ ID NO:321 and a light chain comprising the amino acid sequence of SEQ ID NO:322. The activatable antibody having heavy chain SEQ ID No: 320 and light chain SEQ ID No. 322 is referred to as TY22404. In some embodiments, the activatable antibody comprises a heavy chain comprising the amino acid sequence of SEQ ID NO:320 and a light chain comprising the amino acid sequence of SEQ ID NO:322. In some embodiments, the activatable anti-CTLA4 antibody comprises a heavy chain having at least 90% (e.g., at least 92%, at least 93%, at least 94%, at least 95%, at least 96%, at least 97%, at least 98% or at least 99%) sequence identity to the amino acid sequence of SEQ ID NO:320. In some embodiments, the activatable anti-CTLA4 antibody comprises a light chain having at least 90% (e.g., at least 92%, at least 93%, at least 94%, at least 95%, at least 96%, at least 97%, at least 98% or at least 99%) sequence identity to the amino acid sequence of SEQ ID NO:321. In some embodiments, the activatable anti-CTLA4 antibody comprises a heavy chain having at least 90% (e.g., at least 92%, at least 93%, at least 94%, at least 95%, at least 96%, at least 97%, at least 98% or at least 99%) sequence identity to the amino acid sequence of SEQ ID NO:320. In some embodiments, the activatable anti-CTLA4 antibody comprises a light chain having at least 90% (e.g., at least 92%, at least 93%, at least 94%, at least 95%, at least 96%, at least 97%, at least 98% or at least 99%) sequence identity to the amino acid sequence of SEQ ID NO:322. In some embodiments, the activatable antibody is TY22404.

In some embodiments, an activatable antibody of the present disclosure is generally considered to be an “activatable” binding polypeptide when binding affinity of the polypeptide to CTLA4 (e.g., human CTLA4) increases by at least about 2-fold (e.g., at least about 2-fold, at least about 2.5-fold, at least about 3, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, at least about 10-fold, at least about 25-fold, at least about 50-fold, at least about 75-fold, at least about 100-fold, at least about 250-fold, at least about 500-fold, at least about 750-fold, or at least about 1000-fold, or more) after activation of the activatable antibody as compared to prior to activation of the activatable antibody (e.g., after activation by treatment with one or more proteases that cleave within the cleavable moiety (CM), after activation by a change in pH (increased or decreased), after activation by a temperature shift (increased or decreased), after activation by being contacted with a second molecule (such as a small molecule), etc.). In some embodiments, an activatable antibody of the present disclosure is generally considered “activatable” if the EC₅₀ of the activatable antibody decreases by at least about 2-fold (e.g., at least about 2-fold, at least about 2.5-fold, at least about 3, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, at least about 10-fold, at least about 25-fold, at least about 50-fold, at least about 75-fold, at least about 100-fold, at least about 250-fold, at least about 500-fold, at least about 750-fold, or at least about 1000-fold, or more) after “activation” (e.g., as measured by an ELISA or FACS assay; see the examples below). In some embodiments, an activatable antibody of the present disclosure is generally considered “activatable” if the EC₅₀ of the polypeptide decreases by at least about 2-fold after treatment with a protease that cleaves within the cleavable moiety (CM) (e.g., as measured by an ELISA or FACS assay; see the examples below).

In some embodiments, when the masking moiety (MM) is bound to the target binding moiety (TBM) of the activatable antibody, the K_{D} of the activatable antibody for CTLA4 is about 2 (e.g., about 2, about 2.5, about 3, about 3.5 about 4, about 4.5, about 5, about 5.5, about 6, about 6.5, about 7, about 7.5, about 8, about 8.5, about 9, about 9.5, about 10, about 25, about 50, about 75, about 100, about 250, about 500, about 750, or about 1000 or more) times greater than when the masking moiety (MM) is not bound to the target binding moiety (TBM) (e.g., after “activation” of the activatable antibody (such as after protease treatment to cleave within the cleavable moiety (CM))) and/or than the K_{D} of the parental antibody for CTLA4. Methods of measuring affinity are known in the art, including, for example, by the methods described in the Examples below).

In some embodiments, when the masking moiety is bound to the target binding moiety of the activatable antibody, the K_{D} of the activatable antibody for CTLA4 is reduced by at least about 25% (e.g., at least about 25%, at least about 30%, at least about 40%, at least about 50%, at least about 60%, at least about 70%, at least about 80%, at least about 90%, at least about 95%, at least about 99%) relative to when the masking moiety is not bound to the target binding moiety (e.g., after “activation” of the activatable antibody (such as after protease treatment to cleave within the cleavable moiety (CM))) and/or relative to the K_{D} of the parental antibody for CTLA4. Methods of measuring affinity are known in the art, including, for example, by the methods described in the Examples below).

In some embodiments, the masking moiety sterically hinders binding of the activatable antibody to CTLA4 and/or allosterically hinders binding of the activatable antibody to CTLA4. In some embodiments, the masking moiety does not comprise an amino acid sequence of a natural binding partner of the activatable antibody and/or parental antibody.

In some embodiments, the dissociation constant of the masking moiety for the target binding moiety is greater than the dissociation constant for the activatable antibody for CTLA4 (when activated). In some embodiments, the dissociation constant of the masking moiety for the target binding moiety is about 2 (e.g., about 2, about 2.5, about 3, about 3.5 about 4, about 4.5, about 5, about 5.5, about 6, about 6.5, about 7, about 7.5, about 8, about 8.5, about 9, about 9.5, about 10, about 25, about 50, about 75, about 100, about 250, about 500, about 750, or about 1000 or more) times greater than the dissociation constant for the activatable antibody for CTLA4 (when activated). In some embodiments, the dissociation constant of the masking moiety for the target binding moiety is about equal to the dissociation constant for the activatable antibody for CTLA4 (when activated).

The activatable antibodies described herein may be further modified. In some embodiments, the activatable antibodies are linked to an additional molecular entity. Examples of additional molecular entities include pharmaceutical agents, peptides or proteins, detection agent or labels, and antibodies.

In some embodiments, an activatable antibody of the present disclosure is linked to a pharmaceutical agent. Examples of pharmaceutical agents include cytotoxic agents or other cancer therapeutic agents, and radioactive isotopes. Specific examples of cytotoxic agents include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents also include, for example, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). Examples of radioactive isotopes that can be conjugated to antibodies for use diagnostically or therapeutically include, but are not limited to, iodine¹³¹, indium¹¹¹, yttrium⁹⁰ and lutetium¹⁷⁷. Methods for linking a polypeptide to a pharmaceutical agent are known in the art, such as using various linker technologies. Examples of linker types include hydrazones, thioethers, esters, disulfides and peptide-containing linkers. For further discussion of linkers and methods for linking therapeutic agents to antibodies see e.g., Saito et al., Adv. Drug Deliv. Rev. 55:199-215 (2003); Trail, et al., Cancer Immunol. Immunother. 52:328-337 (2003); Payne, Cancer Cell 3:207-212 (2003); Allen, Nat. Rev. Cancer 2:750-763 (2002); Pastan and Kreitman, Curr. Opin. Investig. Drugs 3:1089-1091 (2002); Senter and Springer (2001) Adv. Drug Deliv. Rev. 53:247-264.

### IV. Combination Therapies with PD-1 Antagonists

In some embodiments, an activatable antibody of the disclosure is administered in combination with a PD-1 antagonist. In one embodiment, the PD-1 antagonist useful in the treatment, medicaments and uses of the present invention include a monoclonal antibody (mAb), or antigen binding fragment thereof, that specifically binds to PD-1 or PD-L1, and preferably specifically binds to human PD-1 or human PD-L1. The mAb may be a human antibody, a humanized antibody or a chimeric antibody, and may include a human constant region. In some embodiments the human constant region is selected from the group consisting of IgG1, IgG2, IgG3 and IgG4 constant regions, and in some embodiments, the human constant region is an IgG1 or IgG4 constant region. In some embodiments, the antigen binding fragment is selected from the group consisting of Fab, Fab′-SH, F(ab′)2, scFv and Fv fragments.

Examples of mAbs that bind to human PD-1, and useful in the treatment method, medicaments and uses of the present invention, are described in U.S. Pat. Nos. 7,488,802, 7,521,051, 8,008,449, 8,354,509, and 8,168,757, and International application publn. nos. WO2004/004771, WO2004/072286, WO2004/056875, US2011/0271358, and WO 2008/156712. Specific anti-human PD-1 mAbs useful as the PD-1 antagonist in the treatment method, medicaments and uses of the present invention include: a humanized IgG4 mAb with the structure described in WHO Drug Information, Vol. 27, No. 2, pages 161-162 (2013) and that comprises the heavy and light chain amino acid sequences shown in Table B; nivolumab (BMS-936558), a human IgG4 mAb with the structure described in WHO Drug Information, Vol. 27, No. 1, pages 68-69 (2013); the humanized antibodies h409A11, h409A16 and h409A17, which are described in WO2008/156712, and AMP-514, which is being developed by MedImmune; cemiplimab; camrelizumab; sintilimab; tislelizumab; and toripalimab. Additional anti-PD-1 antibodies contemplated for use herein include MED10680 (U.S. Pat. No. 8,609,089), BGB-A317 (U.S. Patent publ. no. 2015/0079109), INCSHR1210 (SHR-1210) (PCT International application publ. no. WO2015/085847), REGN-2810 (PCT International application publ. no. WO2015/112800), PDR001 (PCT International application publ. no. WO2015/112900), TSR-042 (ANBO11) (PCT International application publ. no. WO2014/179664) and STI-1110 (PCT International application publ. no. WO2014/194302).

In particular embodiments, an activatable antibody of the disclosure is administered in combination with toripalimab. In some embodiments, provided herein is a method of treating a cancer in a subject, comprising administering to the subject: (a) an effective amount of an activatable antibody, wherein the activatable antibody of the disclosure comprising: a polypeptide comprising, from N-terminus to C-terminus, a masking moiety (MM), a cleavable moiety (CM), and an anti-CTLA4 antibody as described herein, wherein the MM comprises an amino acid sequence EVGSYPNPSSDCVPYYYACAY (SEQ ID NO:192), and the cleavable moiety comprises an amino acid sequence SGRSAGGGGTPLGLAGSGGS (SEQ ID NO:221); and (b) an effective amount of toripalimab. The MM and CM, from N-terminus to C-terminus comprises an amino acid sequence EVGSYPNPSSDCVPYYYACAY SGRSAGGGGTPLGLAGSGGS (SEQ ID NO:200). In particular embodiments, the MM and the CM are covalently attached to the N-terminus of the light chain of the anti-CTLA4 antibody. In some embodiments, the MM and CM, from N-terminus to C-terminus comprises an amino acid sequence that has at least 90% or at least 95% sequence identity to SEQ ID NO:200. In some embodiments, TY22404 is administered in combination with toripalimab.

In some embodiments, the activatable antibody (e.g., TY22404) is administered at a dose of from about 3 mg/kg to about 20 mg/kg or from about 6 mg/kg to about 10 mg/kg once every three to six weeks (e.g., once every three weeks, once every four weeks, once every five weeks or once every six weeks). In some such embodiments, toripalimab is administered at a dose of from about 200 mg to about 400 mg once every three weeks. In another embodiment, toripalimab is administered at a dose of about 240 mg once every three weeks. In another embodiment, toripalimab is administered at a dose of from about 300 mg to about 600 mg once every six weeks. In another embodiment, toripalimab is administered at a dose of about 480 mg once every six weeks.

In one embodiment, the disclosure provides a method of treating a cancer in a subject, comprising administering to the subject an effective amount of an activatable anti-CTLA4 antibody described above (e.g., TY22404) in combination with toripalimab, wherein the anti-CTLA4 antibody is administered at a dose of from about 3 mg/kg to about 10 mg/kg. In some embodiments, the anti-CTLA4 antibody (e.g., TY22404) is administered at a dose of about 3 mg/kg. In some embodiments, the anti-CTLA4 antibody (e.g., TY22404) is administered at a dose of about 5 mg/kg. In some embodiments, the anti-CTLA4 antibody (e.g., TY22404) is administered at a dose of about 6 mg/kg. In some embodiments, the anti-CTLA4 antibody (e.g., TY22404) is administered at a dose of about 8 mg/kg. In some embodiments, the anti-CTLA4 antibody (e.g., TY22404) is administered at a dose of about 10 mg/kg. In any of the foregoing embodiments, toripalimab may be administered at a dose of from about 100 mg to about 400 mg, or from about 200 mg to about 300 mg, or from about 230 mg to about 250 mg. In some embodiments, toripalimab is administered at a dose of about 240 mg. In particular embodiments, the activatable CTLA4 antibody (e.g., TY22404) and 240 mg toripalimab are both administered once every three weeks or once every six weeks. On any of the foregoing embodiments, toripalimab can be administered in combination with the activatable anti-CTLA4 antibody on the same day of a particular dosing regimen or on different days of a particular regimen. In some embodiments, both the activatable anti-CTLA4 antibody and toripalimab are administered on the first day of a three week or six week dosing regimen.

In some embodiments, the activatable anti-CTLA4 antibody (e.g., TY22404) and toripalimab are administered to a patient in need thereof, wherein the activatable anti-CTLA4 antibody (e.g., TY22404) is administered at a dose of 6 mg/kg once every three weeks and the toripalimab is administered at a dose of 240 mg/kg once every three weeks. In some embodiments, the activatable anti-CTLA4 antibody and toripalimab are administered concurrently.

In some embodiments, the activatable anti-CTLA4 antibody (e.g., TY22404) and toripalimab are administered to a patient in need thereof, wherein the activatable anti-CTLA4 antibody (e.g., TY22404) is administered at a dose of 10 mg/kg once every three weeks and the toripalimab is administered at a dose of 200 mg/kg once every three weeks. In some embodiments, the activatable anti-CTLA4 antibody and toripalimab are administered concurrently.

In some embodiments, the activatable anti-CTLA4 antibody (e.g., TY22404) and toripalimab are administered to a patient in need thereof, wherein the activatable anti-CTLA4 antibody (e.g., TY22404) is administered at a dose of 20 mg/kg once every three weeks and the toripalimab is administered at a dose of 200 mg/kg once every three weeks. In some embodiments, the activatable anti-CTLA4 antibody and toripalimab are administered concurrently.

In some embodiments, the activatable anti-CTLA4 antibody (e.g., TY22404) and toripalimab are administered to a patient in need thereof, wherein the activatable anti-CTLA4 antibody (e.g., TY22404) is administered at a dose of 6 mg/kg once every six weeks and the toripalimab is administered at a dose of 240 mg/kg once every six weeks. In some embodiments, the activatable anti-CTLA4 antibody and toripalimab are administered concurrently.

In some embodiments, the activatable anti-CTLA4 antibody (e.g., TY22404) and toripalimab are administered to a patient in need thereof, wherein the activatable anti-CTLA4 antibody (e.g., TY22404) is administered at a dose of 10 mg/kg once every six weeks and the toripalimab is administered at a dose of 200 mg/kg once every six weeks. In some embodiments, the activatable anti-CTLA4 antibody and toripalimab are administered concurrently.

In some embodiments, the activatable anti-CTLA4 antibody (e.g., TY22404) and toripalimab are administered to a patient in need thereof, wherein the activatable anti-CTLA4 antibody (e.g., TY22404) is administered at a dose of 20 mg/kg once every six weeks and the toripalimab is administered at a dose of 200 mg/kg once every six weeks. In some embodiments, the activatable anti-CTLA4 antibody and toripalimab are administered concurrently.

In some embodiments, the activatable anti-CTLA4 antibody is administered intravenously. In some embodiments, the activatable anti-CTLA4 antibody and toripalimab are both administered intravenously. In some embodiments, the activatable anti-CTLA4 antibody is administered subcutaneously. In some embodiments, the activatable anti-CTLA4 antibody and toripalimab are administered intravenously or subcutaneously once every three weeks. In some embodiments, the activatable anti-CTLA4 antibody and toripalimab administered intravenously or subcutaneously once every six weeks. In some embodiments, the subject receives at least 4 cycles of treatment with the activatable anti-CTLA4 antibody and toripalimab. In some embodiments, the subject further receives a maintenance treatment comprising administering to the subject an effective amount of the activatable anti-CTLA4 antibody about once every four weeks to about once every twelve weeks (e.g., once every 4, 6, 8, 10, or 12 weeks). In some embodiments, the doses of the activatable anti-CTLA4 antibody and toripalimab may be administered at the same time. In other embodiments, the doses of the activatable anti-CTLA4 antibody and toripalimab may be administered. For instance, in some embodiments, the activatable anti-CTLA4 antibody and toripalimab are both administered intravenously. In some embodiments, the activatable anti-CTLA4 antibody is administered subcutaneously. In some embodiments, the activatable anti-CTLA4 antibody and toripalimab are administered intravenously or subcutaneously once every three weeks. In some embodiments, the activatable anti-CTLA4 antibody and toripalimab administered intravenously or subcutaneously once every six weeks. In some embodiments, the subject receives at least 4 cycles of treatment with the activatable anti-CTLA4 antibody and toripalimab. In some embodiments, the subject further receives a maintenance treatment comprising administering to the subject an effective amount of the activatable anti-CTLA4 antibody about once every four weeks to about once every twelve weeks (e.g., once every 4, 6, 8, 10, or 12 weeks). In some embodiments, the doses of the activatable anti-CTLA4 antibody and toripalimab may be administered at the same time. In other embodiments, the doses of the activatable anti-CTLA4 antibody and toripalimab may be administered. For instance, toripalimab may be administered from about 0.5 hours to about 5 hours prior to or following administration of the activatable anti-CTLA4 antibody on day 1 of the dosing schedule (e.g., three week dosing schedule). may be administered from about 0.5 hours to about 5 hours prior to or following administration of the activatable anti-CTLA4 antibody on day 1 of the dosing schedule (e.g., three week dosing schedule). are both administered intravenously. In some embodiments, the activatable anti-CTLA4 antibody is administered subcutaneously. In some embodiments, the activatable anti-CTLA4 antibody and In some embodiments, the activatable anti-CTLA4 antibody and toripalimab are both administered intravenously. In some embodiments, the activatable anti-CTLA4 antibody is administered subcutaneously. In some embodiments, the activatable anti-CTLA4 antibody and toripalimab are administered intravenously or subcutaneously once every three weeks. In some embodiments, the activatable anti-CTLA4 antibody and toripalimab administered intravenously or subcutaneously once every six weeks. In some embodiments, the subject receives at least 4 cycles of treatment with the activatable anti-CTLA4 antibody and toripalimab. In some embodiments, the subject further receives a maintenance treatment comprising administering to the subject an effective amount of the activatable anti-CTLA4 antibody about once every four weeks to about once every twelve weeks (e.g., once every 4, 6, 8, 10, or 12 weeks). In some embodiments, the doses of the activatable anti-CTLA4 antibody and toripalimab may be administered at the same time. In other embodiments, the doses of the activatable anti-CTLA4 antibody and toripalimab may be administered. For instance, toripalimab may be administered from about 0.5 hours to about 5 hours prior to or following administration of the activatable anti-CTLA4 antibody on day 1 of the dosing schedule (e.g., three week dosing schedule). are administered intravenously or subcutaneously once every three weeks. In some embodiments, the activatable anti-CTLA4 antibody and toripalimab administered intravenously or subcutaneously once every six weeks. In some embodiments, the subject receives at least 4 cycles of treatment with the activatable anti-CTLA4 antibody and toripalimab. In some embodiments, the subject further receives a maintenance treatment comprising administering to the subject an effective amount of the activatable anti-CTLA4 antibody about once every four weeks to about once every twelve weeks (e.g., once every 4, 6, 8, 10, or 12 weeks). In some embodiments, the doses of the activatable anti-CTLA4 antibody and toripalimab may be administered at the same time. In other embodiments, the doses of the activatable anti-CTLA4 antibody and toripalimab may be administered. For instance, toripalimab may be administered from about 0.5 hours to about 5 hours prior to or following administration of the activatable anti-CTLA4 antibody on day 1 of the dosing schedule (e.g., three week dosing schedule).

### V. Pharmaceutical Compositions, Kits, and Articles of Manufacture

In other aspects, the present application provides a composition comprising any one of the anti-CTLA4 antibodies (e.g., activatable antibodies) described herein. In some embodiments, the composition is a pharmaceutical composition comprising the anti-CTLA4 antibody (e.g., activatable antibodies) and a pharmaceutically acceptable carrier. In some embodiments, provided herein is a composition comprising one or more additional therapeutic agents (e.g., an anti-PD-1 antibody, an anti-PD-L1 antibody). The compositions can be prepared by conventional methods known in the art.

The term “pharmaceutically acceptable carrier” refers to any inactive substance that is suitable for use in a formulation for the delivery of an active agent (e.g., the anti-CTLA4 antibody). A carrier may be an anti-adherent, binder, coating, disintegrant, filler or diluent, preservative (such as antioxidant, antibacterial, or antifungal agent), sweetener, absorption delaying agent, wetting agent, emulsifying agent, buffer, and the like. Examples of suitable pharmaceutically acceptable carriers include water, ethanol, polyols (such as glycerol, propylene glycol, polyethylene glycol, and the like) dextrose, vegetable oils (such as olive oil), saline, buffer, buffered saline, and isotonic agents such as sugars, polyalcohols, sorbitol, and sodium chloride. The compositions may be in any suitable forms, such as liquid, semi-solid, and solid dosage forms. Examples of liquid dosage forms include solution (e.g., injectable and infusible solutions), microemulsion, liposome, dispersion, or suspension. Examples of solid dosage forms include tablet, pill, capsule, microcapsule, and powder. A particular form of the composition suitable for delivering an anti-CTLA4 antibody is a sterile liquid, such as a solution, suspension, or dispersion, for injection or infusion. Sterile solutions can be prepared by incorporating the antibody in the required amount in an appropriate carrier, followed by sterilization microfiltration. Generally, dispersions are prepared by incorporating the antibody into a sterile vehicle that contains a basic dispersion medium and other carriers. In the case of sterile powders for the preparation of sterile liquid, methods of preparation include vacuum drying and freeze-drying (lyophilization) to yield a powder of the active ingredient plus any additional desired ingredient from a previously sterile-filtered solution thereof. The various dosage forms of the compositions can be prepared by conventional techniques known in the art.

The relative amount of an anti-CTLA4 antibody included in the composition will vary depending upon a number of factors, such as the specific anti-CTLA4 antibody and carriers used, dosage form, and desired release and pharmacodynamic characteristics. The amount of an anti-CTLA4 antibody in a single dosage form will generally be that amount which produces a therapeutic effect, but may also be a lesser amount. Generally, this amount will range from about 0.01 percent to about 99 percent, from about 0.1 percent to about 70 percent, or from about 1 percent to about 30 percent relative to the total weight of the dosage form.

In addition to the anti-CTLA4 antibody, one or more additional therapeutic agents may be included in the composition. Examples of additional therapeutic agents are described herein in the “Methods of Treatment” section. The suitable amount of the additional therapeutic agent to be included in the composition can be readily selected by a person skilled in the art, and will vary depending on a number of factors, such as the particular agent and carriers used, dosage form, and desired release and pharmacodynamic characteristics. The amount of the additional therapeutic agent included in a single dosage form will generally be that amount of the agent, which produces a therapeutic effect, but may be a lesser amount as well.

In some embodiments, there is provided an article of manufacture comprising materials useful for the treatment of a cancer. The article of manufacture can comprise a container and a label or package insert on or associated with the container. Suitable containers include, for example, bottles, vials, syringes, etc. The containers may be formed from a variety of materials such as glass or plastic. Generally, the container holds a composition, which is effective for treating a cancer, described herein, and may have a sterile access port (for example, the container may be an intravenous solution bag or a vial having a stopper pierceable by a hypodermic injection needle). Package insert refers to instructions customarily included in commercial packages of therapeutic products that contain information about the indications, usage, dosage, administration, contraindications and/or warnings concerning the use of such therapeutic products. In some embodiments, the package insert indicates that the composition is used for treating a cancer. The label or package insert may further comprise instructions for administering the composition to a patient.

Additionally, the article of manufacture may further comprise a second container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Kits are also provided that are useful for various purposes, e.g., for treatment of a cancer described herein, optionally in combination with the articles of manufacture. Kits of the present application include one or more containers comprising any one of the compositions described herein (or unit dosage form and/or article of manufacture). In some embodiments, the kit further comprises other agents (e.g., one or more additional therapeutic agents) and/or instructions for use in accordance with any of the methods described herein. The kit may further comprise a description of selection of individuals suitable for treatment. Instructions supplied in the kits of the present application are typically written instructions on a label or package insert (e.g., a paper sheet included in the kit), but machine-readable instructions (e.g., instructions carried on a magnetic or optical storage disk) are also acceptable.

For example, in some embodiments, there is provided a kit comprising a pharmaceutical composition comprising any one of the anti-CTLA4 antibodies described herein and a pharmaceutically acceptable carrier; and instructions for administering the pharmaceutical composition to a subject having a cancer. In some embodiments, the kit further comprises a pharmaceutical composition comprising an additional therapeutic agent, such as a chemotherapeutic agent. In some embodiments, the kit further comprises a pharmaceutical composition comprising an anti-PD-1 antibody. In some embodiments, the kit comprises one or more assays or reagents thereof for determining a level of one or more biomarkers described herein (e.g., CD8+ T cells, CD4+ T cells, CD8+Tₑₘ cells, CD4+Tₑₘ cells, T_{reg} cells, a ratio of CD8+Tₑₘ cells to T_{reg} cells, a ratio of CD4+Tₑₘ cells to T_{reg} cells, NK cells, B cells).

The kits of the present application are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The containers may be unit doses, bulk packages (e.g., multi-dose packages) or sub-unit doses. Kits may also include multiple unit doses of the pharmaceutical compositions and instructions for use and packaged in quantities sufficient for storage and use in pharmacies, for example, hospital pharmacies and compounding pharmacies.

The foregoing written description is considered to be sufficient to enable one skilled in the art to practice the present disclosure. The following Examples are offered for illustrative purposes only, and are not intended to limit the scope of the present disclosure in any way. Indeed, various modifications of the present disclosure in addition to those shown and described herein will become apparent to those skilled in the art from the foregoing description and fall within the scope of the appended claims.

### EXAMPLES

The invention can be further understood by reference to the following examples, which are provided by way of illustration and are not meant to be limiting.

### Example 1. A First-In-Human (FIH), Open-Label, Phase I, Dose Escalation Study of TY22404, Ty22404 in Combination with Anti-PD-1 Antibody in Patients with Advanced/Metastatic Solid Tumors

### Method

This is a first-in-human, Phase I, open-label, multicenter, sequential dose escalation and dose-expansion study to evaluate the safety, tolerability, PK, and preliminary efficacy of TY22404 monotherapy, and TY22404-toripalimab combination regimens in patients with advanced/metastatic solid tumors. The study consists of Part 1: TY22404 monotherapy dose escalation followed by a biopsy cohort and expansion (concurrently); Part 2: TY22404-toripalimab combination dose escalation and expansion. The objective of the study is to assess the safety and tolerability of TY22404 monotherapy at escalating dose levels, and TY22404 in combination with toripalimab, in adult patients with advanced/metastatic solid tumors, who have exhausted their treatment options.

Dose Escalation. TY22404 monotherapy dose escalation will be traditional 3+3 cohort design. Three to six patients will be enrolled to each of up to 6 successive escalating dose levels of 0.1, ≤0.3, ≤1, ≤3, ≤10, and ≤20 mg/kg every 3 weeks (Q3W). The dose levels or the dosing interval may be adjusted during the study based on observed safety data but will not exceed 20 mg/kg as the predetermined MAD.

Combination Dose Escalation. Combination therapy will commence at a dose level lower than the cleared dose from the monotherapy dose escalation arms and approved by the SRC. The projected RP2D is 20 mg/kg every Q3W based on nonclinical data. To maximize patient safety, the starting dose in the combination dose escalation arms will be a dose lower (such as 10 mg/kg Q3W) than the RP2D. Enrollment to the combination therapy dose escalation arms (may enroll concurrently) may occur before defining the RP2D/MAD in the monotherapy dose escalation arms. A modified Toxicity Probability Interval (mTPI) design with a target DLT rate of approximately 30% will be applied for dose escalation and confirmation to determine a RP2D for TY22404 in combination with toripalimab. The dose escalation includes dose escalation of combination regimens and 3 dose escalation levels for each combination regimen as below:

| | Dose Level | TY22404 | Toripalimab |
|---|---|---|---|
| Design | (DL) | (mg/kg, Q3W) | (mg, Q3W) |
| mTPI | DL1 | SRC determined starting | 240 |
| design | | combination dose | |
| | DL2 | SRC determined starting | 240 |
| | | combination dose + 1 | |
| | DL3^{a} | ≤20 | 240 |

DL = dose level;
mTPI = modified Toxicity Probability Interval;
SRC = safety review committee;
Q3W = every 3 weeks
^{a}DL3 denotes dose level 3. The number of the dose level will be determinred based on data generated during combination therapy dose escalation

Key Inclusion Criteria. Patients with advanced/metastatic solid tumors with ECOG≤1 and at least one measurable lesion per RECIST1.1. Imaging was performed every 6 weeks for the first 4 cycles, then every 9 weeks afterwards. Tumor response was investigator-determined using RECIST1.1 and iRECIST. Prior treatment by anti-PD-1 or anti-CTLA-4 therapy are allowed.

Endpoints. The primary endpoints are safety and tolerability, determine maximum administered dose (MAD), maximum tolerated dose (MTD) and recommended Phase 2 doses (RP2Ds). The secondary endpoints is PK, anti-drug antibodies (ADA), ORR, DCR, DOR, PFS and OS per RECIST1.1. Evaluation. Imaging was performed every 6 weeks for the first 4 cycles, then every 9 weeks afterwards

### TY22404 Monotherapy Initial Interim Results

26 subjects had been treated by TY22404 monotherapy Subjects were heavily pre-treated. Tumor types included breast cancer, cholangiocarcinoma, colorectal cancer, epithelial ovarian cancer, glioblastoma, hepatocellular carcinoma, melanoma, non-small cell lung cancer, pancreatic adenocarcinoma, renal cell carcinoma, uveal melanoma, and others.

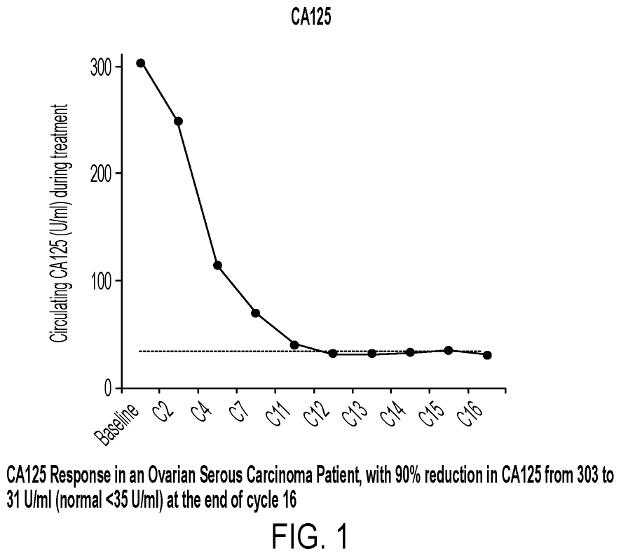

TY22404 monotherapy (N=26) is well tolerated with no dose-limiting toxicities up to 20 mg/kg. The most frequent treatment-related adverse event (TRAE) (>10%) was fatigue (12%), pruritus (12%), rash (12%) and diarrhea (12%) (Table 2). TY22404 demonstrates single-agent anti-tumor efficacy with disease control rate of 39% among 23 evaluable patients (FIG. 1A-B.).

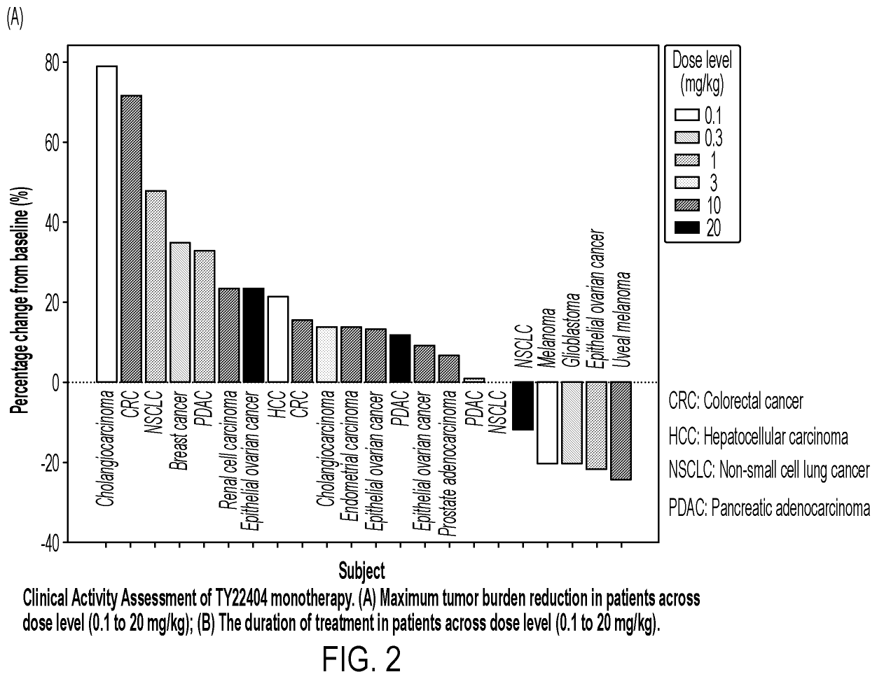

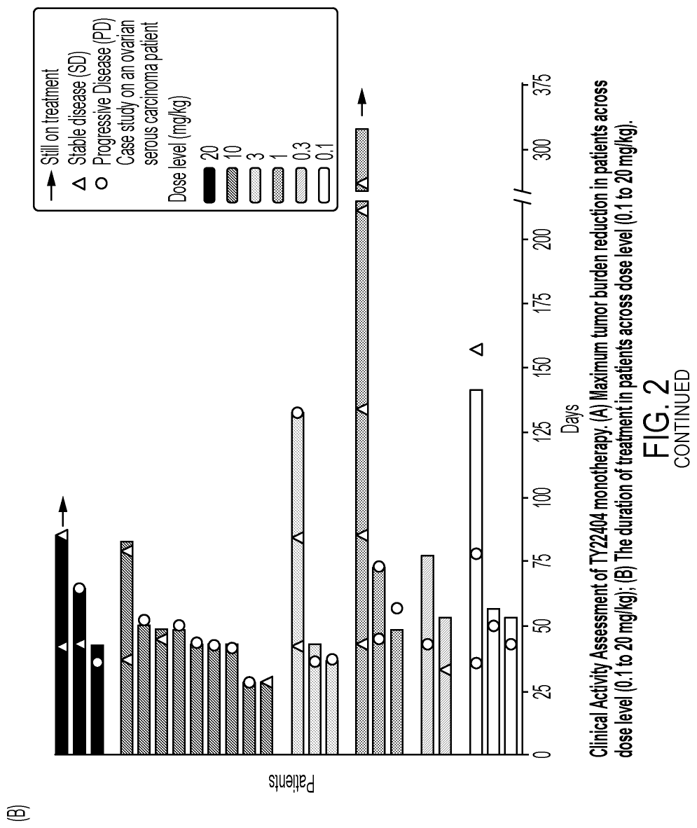

A patient previously received curative salpingo-oophorectomy and 5 lines of systemic therapies showed continuous response after treatment of TY22404 at 1 mg/kg, the patient showed 90% reduction in CA125 from 303 to 31 U/ml (normal <35 U/ml) (FIG. 2), and 22% decrease in target lesions at the end of cycle 16 (Table 3). The treatment is still ongoing in the 18^{th} cycles.

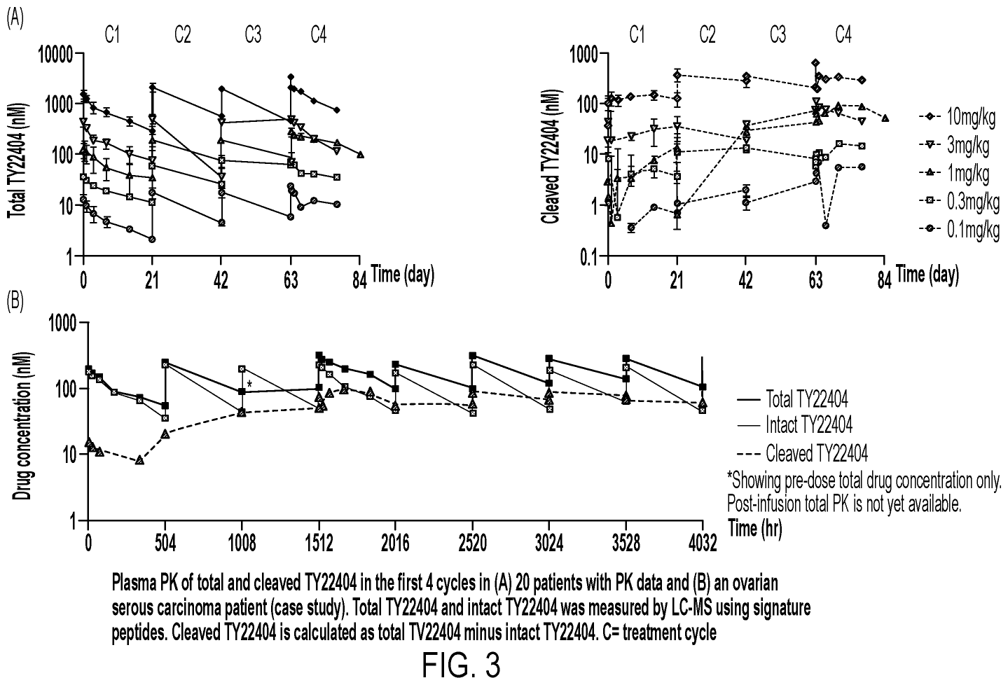

Plasma pharmacokinetics (PK) of Total and Intact drug determined by LC-MS using respective signature peptides were approximately linear with dose and the calculated Cleaved (i.e. activated) TY22404 accumulated on average ˜3-fold (e.g., Cycle 4 vs. Cycle 1) during repeat dosing (FIG. 3A-B). Non-compartmental analysis (NCA) of PK suggested a ˜1.5-fold mean increase in the terminal half-life of Total TY22404 in circulation, compared with its parental Ab TY21580. The circulating Cleaved drug PK is reflective of prolonged exposures of activated TY22404 in the tumor microenvironment. PK modeling further predicted target saturation at ˜6-10 mg/kg at steady state.

TABLE 1
Baseline Characteristics

| Characteristics | N = 26 |
|---|---|
| Age (Years), median (range) | 64 (41, 84) |
| Female, n (%) | 14 (54%) |

Race, n (%)

| | |
|---|---|
| Caucasian | 25 (96%) |
| Asian | 1 (4%) |

ECOG, n (%)

| | |
|---|---|
| 0 | 12 (46%) |
| 1 | 14 (54%) |

Prior lines of therapy, n (%)

| | |
|---|---|
| 3 lines or above | 15 (58%) |
| Prior immunotherapy, n (%) | 11 (42%) |

TY22404 dose administered

| | |
|---|---|
| Median (range) | 2 (1, 18) |

TABLE 2
Summary of TRAE in patients treated with TY22404 monotherapy

| | All Monotherapy | | | |
|---|---|---|---|---|
| Summary | (N = 26) | G1 | G2 | G3-5 |
| Subjects with any TEAE | 10 (38.4%) | 6 (23.1%) | 4 (15.4%) | 0 |
| Fatigue | 3 (11.5%) | 3 (11.5%) | 0 | 0 |
| Pruritus | 3 (11.5%) | 3 (11.5%) | 0 | 0 |
| Rash | 3 (11.5%) | 3 (11.5%) | 0 | 0 |
| Diarrhoea | 3 (11.5%) | 2 (7.7%) | 1 (3.8%) | 0 |
| Headache | 2 (7.7%) | 2 (7.7%) | 0 | 0 |
| Adrenal insufficiency | 1 (3.8%) | 0 | 1 (3.8%) | 0 |
| Arthralgia | 1 (3.8%) | 1 (3.8%) | 0 | 0 |
| Chills | 1 (3.8%) | 1 (3.8%) | 0 | 0 |
| Cough | 1 (3.8%) | 1 (3.8%) | 0 | 0 |
| Decreased appetite | 1 (3.8%) | 0 | 1 (3.8%) | 0 |
| Dry skin | 1 (3.8%) | 1 (3.8%) | 0 | 0 |
| Dyspepsia | 1 (3.8%) | 1 (3.8%) | 0 | 0 |
| Dyspnoea | 1 (3.8%) | 1 (3.8%) | 0 | 0 |
| Hyperhidrosis | 1 (3.8%) | 1 (3.8%) | 0 | 0 |
| Hypothermia | 1 (3.8%) | 1 (3.8%) | 0 | 0 |
| Nausea | 1 (3.8%) | 1 (3.8%) | 0 | 0 |
| Neuropathy peripheral | 1 (3.8%) | 0 | 1 (3.8%) | 0 |
| Pyrexia | 1 (3.8%) | 1 (3.8%) | 0 | 0 |

TABLE 3
Clinical Response in an Ovarian Serous Carcinoma Patient

| | | Baseline | End of C2 | | End of C7 | | End of C16 | |
|---|---|---|---|---|---|---|---|---|
| Target | TL1 - Lymph Node | 17 mm | 13 mm | | 12 mm | | 12 mm | |
| Lesion | TL2 - Lymph Node | 15 mm | 15 mm | | 13 mm | | 13 mm | |
| | Sum, % from baseline | 32 mm | 28 mm | (−13%) | 25 mm | (−22%) | 25 mm | (−22%) |

| Non-Target Lesion | Present | Present | | Present | | Present | |
|---|---|---|---|---|---|---|---|
| New Lesion | NA | No | | No | | No | |
| Overall Response | NA | SD | | SD | | SD | |
| CA-125 (U/ml, % from baseline) | 303 | 249 | (−18%) | 70 | (−77%) | 31 | (−90%) |

### Ty22404 Updated Interim Results

Across all dose levels, 30 patients (Table 4) received TY22404 monotherapy and 20 patients received TY22404 (6 mg/kg Q3W and 10 mg/kg Q3W or Q6W)+Toripalimab (240 mg Q3W). Patients were heavily pre-treated. Tumor types included breast cancer, cholangiocarcinoma, colorectal cancer, epithelial ovarian cancer, glioblastoma, hepatocellular carcinoma, melanoma, non-small cell lung cancer, pancreatic adenocarcinoma, renal cell carcinoma, uveal melanoma, prostate adenocarcinoma, endometrial carcinoma, gastrointestinal stroma tumor (GIST), squamous cell carcinoma (anal, anorectal, penile, or cutaneous), adenocarcinoma and others. These tumor types were included in the study, including “cold tumors” or immune-experienced “hot tumors”.

TABLE 4
Patient Demographics, Characteristics and Drug Exposure

| | TY22404 | TY22404 + Toripalimab |
|---|---|---|
| Characteristics | (N = 30) | (N = 20) |
| Age (years), median (range) | 63.5 (39, 84) | 59.5 (36, 85) |
| Female, n (%) | 16 (53) | 10 (50) |

Race, n (%)

| | | |
|---|---|---|
| Caucasian, n (%) | 27 (90) | 15 (75) |
| Asian, n (%) | 3 (10) | 4 (20) |
| Native American, n (%) | 0 | 0 |
| Aboriginal Australian, n (%) | 0 | 1 (5) |
| ECOG PS, n (%) | | |
| 0 | 16 (53) | 9 (45) |
| 1 | 14 (47) | 11 (55) |

Number of prior lines of treatment, n (%)

| | | |
|---|---|---|
| ≥3 | 16 (53) | 11 (55) |
| Prior immunotherapy, n (%) | 14 (47) | 7 (35) |
| Study treatment cycles | | |
| Median (range) | 2 (1, 25) | 4 (1, 10)* |
| ≥4 cycles, n (%) | 9 (30) | 13 (65)* |

*Study treatment cycles includes TY22404 and/or Toripalimab

### Safety—TY22404 Monotherapy

A shown it Table 5, TY22404 monotherapy is well tolerated with no dose limiting toxicities at doses up to 20 mg/kg with repeat dosing. No >Grade 2 treatment-related adverse events (TRAEs) and no treatment-related serious adverse events (SAEs) have been reported. No patient discontinued study treatment due to TRAE.

TABLE 5
TRAEs reported at any dose level of TY22404 monotherapy

| TY22404 | | All G | G1 | G2 | G3-G5 |
|---|---|---|---|---|---|
| (mg/kg) | N | n (%) | n (%) | n (%) | n (%) |
| All | 30 | 13 (43) | 9 (30) | 4 (13) | 0 |
| 0.1 | 3 | 0 | 0 | 0 | 0 |
| 0.3 | 3 | 2 (67) | 2 (67) | 0 | 0 |
| 1 | 4 | 1 (25) | 0 | 1 (25) | 0 |
| 3 | 3 | 3 (100) | 3 (100) | 0 | 0 |

### Safety—TY22404+Toripalimab Dose Escalation

Three dose escalation cohorts were explored, including 7 patients who received TY22404 6 mg/kg Q3W+Toripalimab 240 mg Q3W, 7 patients who received TY22404 10 mg/kg Q6W+Toripalimab 240 mg Q3W and 9 patients who received TY22404 10 mg/kg Q3W+Toripalimab 240 mg Q3W Table 6). No significant differences in safety were observed across these three dose escalation cohorts. No DLT or >Grade 3 TRAE has been reported. Five patients (21%) experienced G3 TRAEs: Most of G3 TRAEs occurred no earlier than Cycle 4. Three patients experienced treatment-related SAEs, including one G3 hepatitis§, one G3 sepsis‡ and one G3 myocarditis‡ (this led to discontinuation of study).

TABLE 6
TRAEs reported at any dose level of TY22404 + Toripalimab Dose Escalation

| TY22404 | | | G1 | G2 | G3 | G4 or G5 |
|---|---|---|---|---|---|---|
| (mg/kg) | Toripalimab | N | n (%) | n (%) | n (%) | n (%) |
| 6 mg/kg, Q3W | 240 mg Q3W | 7 | 1 (14%) | 1 (14%) | 2 (28%) | 0 |
| 10 mg/kg, Q3W | 240 mg Q3W | 7 | 2 (28%) | 0 | 1 (14%) | 0 |
| 10 mg/kg, Q6W | 240 mg Q3W | 9 | 1 (11%) | 1 (11%) | 2 (22%) | 0 |

### Efficacy—TY22404 Monotherapy

Across all dose levels, disease control rate (DCR)=37% among 27 evaluable patients who had at least one valid post-baseline tumor assessment. Prolonged stable disease was observed in 5 patients, including 14 cycles for a NSCLC patient and 22 cycles for an ovarian carcinoma patient. A 12% reduction in the sum of target lesions was observed in this NSCLC patient who previously progressed on pembrolizumab and docetaxel; this NSCLC patient completed 14 cycles of TY22404 20 mg/kg Q3W with no TRAEs. The ovarian serous carcinoma patient received TY22404 1 mg/kg Q3W and experienced stable disease with a 90% reduction in CA125 and a 22% reduction in the sum of target lesions.

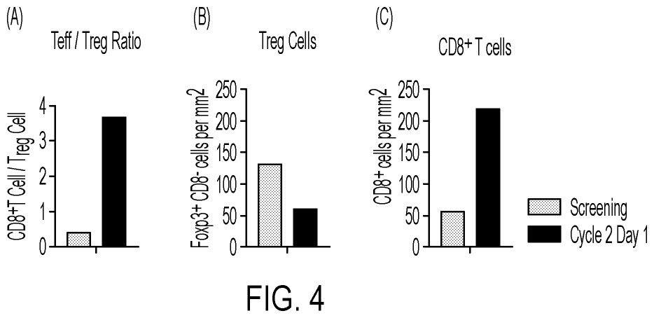

A 39 years-old male patient with stage IIIB hepatocellular carcinoma. The baseline ECOG PS was 0. This patient had previously progressed on anti-PD-L1 therapy atezolizumab in combination with bevacizumab (July 2021-February 2022, PD) as first-line treatment; this patient subsequently received lenvatinib as second-line treatment (March-September 2022, PD). This patient is receiving TY22404 10 mg/kg Q3W in C6 with control of tumor growth (stable disease). As shown in FIG. 4, increased ratio of Teff/Treg (FIG. 4A), with Treg depletion (FIG. 4B) and increased CD8+ T cells (FIG. 4C) was observed in paired tumor biopsies.

### Efficacy—TY22404+Toripalimab Dose Escalation

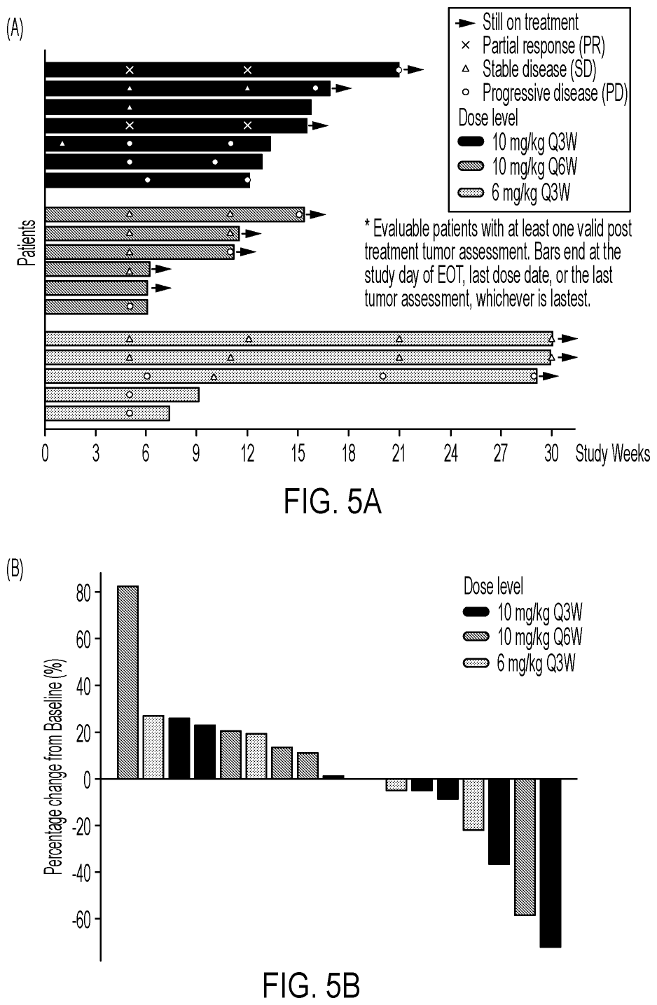

As shown in Table 7 and FIG. 5, the response of 18 evaluable patients in the three dose escalation cohorts who received TY22404 (6 mg/kg Q3W and 10 mg/kg Q3W or Q6W)+Toripalimab (240 mg Q3W) were calculated. Among the 18 evaluable patients, overall ORR=11% and DCR=56%. ORR=28% and DCR=57% was reported among the 7 evaluable patients who received TY22404 10 mg/kg Q3W+TORI 240 mg Q3W. Confirmed partial response (PR) was observed in two patients with penile squamous cell carcinoma (SCC) and anal SCC who were previously treated with chemotherapy. Prolonged stable disease was observed in a PDAC patient with a 5% reduction in the sum of target lesions. A 58% and 21% reduction in the sum of target lesions were observed in two microsatellite stable colorectal cancer (MSS CRC) patients with liver metastasis at baseline, respectively.

TABLE 7
Best Response of TY22404 + Toripalimab Dose Escalation

| | TY22404 | TY22404 |
|---|---|---|
| | (6 mg/kg Q3W) + | (10 mg/kg Q3W or Q6W) + |
| | Toripalimab 240 mg | Toripalimab 240 mg |
| Best Response | Q3W (N* = 5) | Q3W (N* = 13) |
| ORR (%) | 0 | 15% |
| DCR (%) | 40% | 61% |
| CR, n (%) | 0 | 0 |
| PR, n (%) | 0 | 2 (15%) |
| SD, n (%) | 2 (40%) | 6 (46%) |
| PD, n (%) | 3 (60%) | 5 (38%) |

Case Studies: Confirmed PRs in Two Patients with Anal SCC and Penile SCC

As shown in Table 8, confirmed PR with a 36% reduction in the sum of target lesions was observed in a 51 years-old female patient with metastatic anal squamous cell carcinoma (SCC) who received TY22404 10 mg/kg Q3W+toripalimab 240 mg Q3W (treatment is ongoing in Cycle 5 as of this datacut). Her baseline ECOG PS was 0. She previously received fluorouracil (5FU)/mitomycin C+radiation.

TABLE 8
Tumor assessments in a patient with metastatic anal squamous cell carcinoma

| | | Baseline | Week 6 | Week 12 | Week 17 |
|---|---|---|---|---|---|
| Target Lesion | TL1 - Lung | 11 mm | 7 mm | 7 mm | 17 mm |
| | Sum | 11 mm | 7 mm (−36%) | 7 mm (−36%) | 17 mm (54%) |
| Non-Target | Lung | Present | Present | Present | Present |
| Lesion | | | | | |

| | | | | |
|---|---|---|---|---|
| New Lesion | NA | None | None | None |
| Overall Response | NA | PR | PR | PD (iuPD) |

As shown in Table 9, confirmed PR with a 72% reduction in the sum of target lesions was observed in a 64 years-old male with metastatic penile SCC who received TY22404 10 mg/kg Q3W+toripalimab 240 mg Q3W (treatment is ongoing in Cycle 6 as of this datacut). His baseline ECOG PS was 1. This patient previously received partial penectomy, bilateral lymph node dissection, chemotherapy (paclitaxel, ifosfamide, cisplatin) and palliative RT.

TABLE 9
Tumor assessments in a patient with metastatic penile SCC

| | | Baseline | Week 7 | Week 14 |
|---|---|---|---|---|
| Target Lesion | TL1 - Lymph Node | 18 mm | 9 mm | 5 mm |
| | Sum | 18 mm | 9 mm (−50%) | 5 mm (−72%) |
| Non-Target | Thigh | Present | Present | Present |
| Lesion | | | | |

| | | | |
|---|---|---|---|
| New Lesion | NA | None | None |
| Overall Response | NA | PR | PR |

### Case Studies: Promising Potential of TY22404+Toripalimab in “Cold” Gastrointestinal Tumors

As shown in Table 10, a 53 year-old male who had MSS rectosigmoid adenocarcinoma with liver metastasis at baseline received TY22404 10 mg/kg Q6W+toripalimab 240 mg Q3W (treatment is ongoing as of this datacut). His baseline ECOG PS was 0. He previously received curative surgery and palliative surgery for liver metastasis and 3 lines of therapies: Leucovorin+5FU+irinotecan+oxaliplatin, bevacizumab+TAS102 and regorafenib. Mixed response with a 58% reduction in the sum of target lesions was observed at week 17.

TABLE 10
Tumor assessments in a patient with MSS CRC

| | | Baseline | Week 7 | Week 13 | Week 17 |
|---|---|---|---|---|---|
| Target Lesion | TL1 - Lung | 15 mm | 14 mm | 13 mm | 16 mm |
| | TL2 - Lymph | 22 mm | 12 mm | 12 mm | Disappeared |
| | Node | | | | |
| | TL3 - Lymph | 18 mm | 10 mm | 6 mm | Disappeared |
| | Node | | | | |
| | TL4 - Lymph | 19 mm | 14 mm | 15 mm | Disappeared |
| | Node | | | | |
| | TL5 - Liver | 17 mm | 20 mm | 22 mm | 22 mm |
| | Sum | 91 mm | 70 mm | 68 mm | 38 mm |
| | | | (−23%) | (−25%) | (−58%) |
| Non-Target | NTL1 - Lung | Present | Present | Present | Disappeared |
| Lesion | NTL2 - Bone | Present | Present | Present | Present |

| | | | | |
|---|---|---|---|---|
| New Lesion | NA | No | No | Yes |
| Overall Response | NA | SD | SD | PD (iuPD) |

As shown in Table 11, a 74 years-old male with MSS CRC with liver metastasis at baseline received TY22404 6 mg/kg Q3W+toripalimab 240 mg Q3W. His baseline ECOG PS was 1. He previously received 3 lines of therapies: XELOX+bevacizumab; irinotecan+cetuximab; and irinotecan+panitumumab. A 21% reduction in the sum of target lesions was observed (treatment is ongoing as of this datacut).

TABLE 11
Tumor assessments in a patient with MSS CRC

| | | Baseline | Week 8 | Week 14 | Week 24 | Week 33 |
|---|---|---|---|---|---|---|
| Target Lesion | TL1 - Liver | 55 mm | 52 mm | 44 mm | 40 mm | 38 mm |
| | TL2 - Liver | 48 mm | 48 mm | 44 mm | 44 mm | 43 mm |
| | Sum | 103 mm | 100 mm | 88 mm | 84 mm | 81 mm |
| | | | (−3%) | (−15%) | (−18%) | (−21%) |

| | | | | | |
|---|---|---|---|---|---|
| Non-Target Lesion | Present | Present | Present | Present | Present |
| New Lesion | NA | No | No | No | No |
| Overall Response | NA | SD | SD | SD | SD |

As shown in Table 12, a 56 years-old female with pancreatic ductal adenocarcinoma (PDAC) received TY22404 6 mg/kg Q3W+toripalimab 240 mg Q3W. Her baseline ECOG PS was 0. She previously received curative surgery and 4 lines of therapies: gemcitabine/nab-paclitaxel; FOLFIRINOX; oxaliplatin, raltitrexed, irinotecan and leucovorin; and gemcitabine/capecitabin. Prolonged control of tumor growth (stable disease) with a 5% reduction in the sum of target lesions was observed (treatment is ongoing).

TABLE 12
Tumor assessments in a patient with PDAC

| | | Baseline | Week 8 | Week 15 | Week 23 | Week 31 |
|---|---|---|---|---|---|---|
| Target | TL1 - Liver | 23 mm | 22 mm | 23 mm | 23 mm | 17 mm |
| Lesion | TL2 - Liver | 21 mm | 21 mm | 20 mm | 19 mm | 25 mm |
| | Sum | 44 mm | 43 mm | 43 mm | 42 mm | 42 mm |
| | | | (−2%) | (−2%) | (−5%) | (−5%) |

| | | | | | |
|---|---|---|---|---|---|
| Non-Target Lesion | Present | Present | Present | Present | Present |
| New Lesion | NA | No | No | No | No |
| Overall Response | NA | SD | SD | SD | SD |

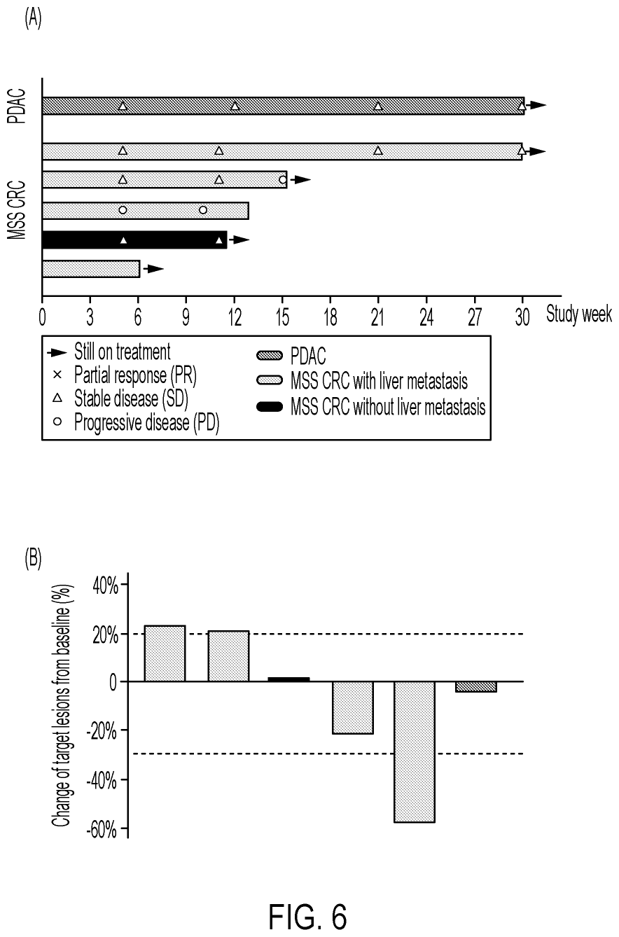

FIGS. 6A-6B show response to TY22404+toripalimab in selected gastrointestinal “cold tumors” as set forth above. FIG. 6A shows a Waterfall plot. FIG. 6B shows a Swimmer plot.

TY22404 monotherapy is well tolerated up to 20 mg/kg Q3W and demonstrated promising efficacy signals in heavily pre-treated patients. Prolonged stable disease was observed in 5 patients. Increased ratio of Teff/Treg, with Treg depletion and increased CD8+ T cells was observed in paired tumor biopsies. The safety profile of TY22404+toripalimab dose escalation demonstrates best-in-class potential in comparison with other anti-CTLA-4 molecules in combination with anti-PD-1 antibody at the similar doses/schedules. TY22404+toripalimab demonstrates encouraging efficacy, including two confirmed PRs, as well as prolonged stable disease and reduced target lesions in “cold” GI tumors. ORR=28% and DCR=57% among the 7 evaluable patients who received TY22404 10 mg/kg Q3W+toripalimab 240 mg Q3W. Prolonged stable disease as well as a 58% and 21% reduction in the sum of target lesion was observed in two MSS CRC patients with liver metastasis at baseline; similar findings were also observed in a PDAC patient with a 5% reduction in the sum of target lesions. Continuous dosing beyond 4 cycles enabled by the favorable safety profile may enable combination with agents beyond anti-PD-1 therapy: a study of TY22404+atezolizumab+bevacizumab (NCT04524871) is being planned. The unique mechanism of Treg depletion may unleash the potential of TY22404 in addressing significant unmet need in “cold tumors”. Dose expansion of TY22404 10 mg/kg+toripalimab is planned in indications including MSS CRC.

EXEMPLARY SEQUENCES
HVR-H1 Activatable anti-CTLA4
SEQ ID NO: 23
YSISSGYHWSWI
HVR-H2 Activatable anti-CTLA4
SEQ ID NO: 35
LARIDWDDDKYYSTSLKSRL
HVR-H3 Activatable anti-CTLA4
SEQ ID NO: 45
ARSYVYFDY
HVR-L1 Activatable anti-CTLA4
SEQ ID NO: 58
RASQSVRGRFLA
HVR-L2 Activatable anti-CTLA4
SEQ ID NO: 66
DASNRATGI
HVR-L3 Activatable anti-CTLA4
SEQ ID NO: 75
YCQQSSSWPPT
Masking Moiety (MM)
SEQ ID NO: 192
EVGSYPNPSSDCVPYYYACAY
Cleavable Moiety (CM)
SEQ ID NO: 221
SGRSAGGGGTPLGLAGSGGS
Masking Moiety (MM) plus Cleavable Moiety (CM)
SEQ ID NO: 200
EVGSYPNPSSDCVPYYYACAYSGRSAGGGGTPLGLAGSGGS
Toripalimab (anti-PD1) HVR-H1
SEQ ID NO: 312
DYEMH
Toripalimab HVR-H2
SEQ ID NO: 313
VIESETGGTAYNQKFKG
Toripalimab HVR-H3
SEQ ID NO: 314
EGITTVATTYYWYFDV
Toripalimab HVR-L1
SEQ ID NO: 315
RSSQSIVHSNGNTYLE
Toripalimab HVR-L2
SEQ ID NO: 316
KVSNRFS
Toripalimab HVR-L3
SEQ ID NO: 317
FQGSHVPLT
Toripalimab VH
SEQ ID NO: 318
QGQLVQSGAEVKKPGASVKVSCKASGYTFTDYEMHWVRQAPIHGLEWIGVIESETGGTAYNQK
FKGRVTITADKSTSTAYMELSSLRSEDTAVYYCAREGITTVATTYYWYFDVWGQGTTVTVSS
Toripalimab VL
SEQ ID NO: 319
DVVMTQSPLSLPVTLGQPASISCRSSQSIVHSNGNTYLEWYLQKPGQSPQLLIYKVSNRFSGVPDR
FSGSGSGTDFTLKISRVEAEDVGVYYCFQGSHVPLTFGQGTKLEIK
Activatable anti-CTLA4 2 full heavy chain (minus C-terminal lysine)
SEQ ID NO: 320
EVQLVESGGGLVQPGGSLRLSCAASGYSISSGYHWSWIRQAPGKGLEWLARIDWDDDKYYSTSL
KSRLTISRDNSKNTLYLQLNSLRAEDTAVYYCARSYVYFDYWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSNKALPAPIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSPG
Activatable anti-CTLA4 2 full heavy chain (with C-terminal lysine)
SEQ ID NO: 321
EVQLVESGGGLVQPGGSLRLSCAASGYSISSGYHWSWIRQAPGKGLEWLARIDWDDDKYYSTSL
KSRLTISRDNSKNTLYLQLNSLRAEDTAVYYCARSYVYFDYWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSNKALPAPIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSPGK
Activatable anti-CTLA4 2 full light chain with N-terminal masking
moiety and linker
SEQ ID NO: 322
EVGSYPNPSSDCVPYYYACAYSGRSAGGGGTPLGLAGSGGSDIQLTQSPSSLSASVGDRVTITCR
ASQSVRGRFLAWYQQKPGKAPKLLIYDASNRATGIPSRFSGSGSGTDFTLTISSLQPEDFATYYCQ
QSSSWPPTFGQGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
Activatable anti-CTLA4 1 full heavy chain (minus C-terminal lysine)
SEQ ID NO: 323
EVQLVESGGGLVQPGGSLRLSCAASGYSISSGYHWSWIRQAPGKGLEWLARIDWDDDKYYSTSL
KSRLTISRDNSKNTLYLQLNSLRAEDTAVYYCARSYVYFDYWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSNKALPAPIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSPG
Activatable anti-CTLA4 1 full heavy chain (with C-terminal lysine)
SEQ ID NO: 324
EVQLVESGGGLVQPGGSLRLSCAASGYSISSGYHWSWIRQAPGKGLEWLARIDWDDDKYYSTSL
KSRLTISRDNSKNTLYLQLNSLRAEDTAVYYCARSYVYFDYWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSNKALPAPIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSPGK
Activatable anti-CTLA4 1 full light chain with N-terminal masking
moiety and linker
SEQ ID NO: 325
EVGSYNFVADSCPDHPYPCSASGRSAGGGGSPLGLAGSGGSDIQLTQSPSSLSASVGDRVTITCRA
SQSVRGRFLAWYQQKPGKAPKLLIYDASNRATGIPSRFSGSGSGTDFTLTISSLQPEDFATYYCQQ
SSSWPPTFGQGTKVEIKRTVAAPSVFIFPPSDEQLKSGTASVVCLLNNFYPREAKVQWKVDNALQ
SGNSQESVTEQDSKDSTYSLSSTLTLSKADYEKHKVYACEVTHQGLSSPVTKSFNRGEC
Activatable anti-CTLA4 3 full heavy chain (minus C-terminal lysine)
SEQ ID NO: 326
EVQLVESGGGLVQPGGSLRLSCAASGYSISSGYHWSWIRQAPGKGLEWLARIDWDDDKYYSTSL
KSRLTISRDNSKNTLYLQLNSLRAEDTAVYYCARSYVYFDYWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSNKALPAPIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSPG
Activatable anti-CTLA4 3 full heavy chain (with C-terminal lysine)
SEQ ID NO: 327
EVQLVESGGGLVQPGGSLRLSCAASGYSISSGYHWSWIRQAPGKGLEWLARIDWDDDKYYSTSL
KSRLTISRDNSKNTLYLQLNSLRAEDTAVYYCARSYVYFDYWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCK
VSNKALPAPIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSPGK
Activatable anti-CTLA4 3 full light chain with N-terminal masking
moiety and linker
SEQ ID NO: 328
EVGSYIVHHSDCDAFYPYCDSSGRSAGGGGSPLGLAGSGGSDIQLTQSPSSLSASVGDRVTITCRA
SQSVRGRFLAWYQQKPGKAPKLLIYDASNRATGIPSRFSGSGSGTDFTLTISSLQPEDFATYYCQQ
SSSWPPTFGQGTKVEIKRTVAAPSVFIFPPSDEQLKSGTASVVCLLNNFYPREAKVQWKVDNALQ
SGNSQESVTEQDSKDSTYSLSSTLTLSKADYEKHKVYACEVTHQGLSSPVTKSFNRGEC

What is claimed is:

1. A method of treating a cancer in a subject, comprising administering to the subject:
(a) an effective amount of an activatable antibody, wherein the activatable antibody comprises an HVR-H1 comprising an amino acid sequence according to a formula YSISSGYHWSWI (SEQ ID NO: 23), an HVR-H2 comprising an amino acid sequence according to a formula LARIDWDDDKYYSTSLKSRL (SEQ ID NO: 35), an HVR-H3 comprising an amino acid sequence according to a formula ARSYVYFDY (SEQ ID NO: 45), an HVR-L1 comprising an amino acid sequence according to the formula RASQSVRGRFLA (SEQ ID NO: 58), an HVR-L2 comprises an amino acid sequence according to a formula DASNRATGI (SEQ ID NO: 66), and an HVR-L3 comprising an amino acid sequence according to a formula YCQQSSSWPPT (SEQ ID NO: 75), and wherein the activatable antibody further comprises: a polypeptide covalently attached to the N-terminus of the light chain of the anti-CTLA4 antibody, said polypeptide comprising, from N-terminus to C-terminus, a masking moiety (MM) and a cleavable moiety (CM), wherein the MM comprises an amino acid sequence EVGSYPNPSSDCVPYYYACAY (SEQ ID NO:192), and the cleavable moiety comprises an amino acid sequence SGRSAGGGGTPLGLAGSGGS (SEQ ID NO:221); and
(b) an effective amount of toripalimab,
wherein the activatable antibody is administered at a dose of from about 6 mg/kg to about 10 mg/kg once every three to six weeks, and wherein the toripalimab is administered at a dose of from about 200 mg to about 400 mg once every three weeks or about 300 mg to about 600 mg once every six weeks.

2. The method of claim 1, wherein the activatable antibody is administered at a dose of about 6 mg/kg once every 3 to 6 weeks.

3. The method of claim 1, wherein the activatable antibody is administered at a dose of about 10 mg/kg once every 3 to 6 weeks.

4. The method of claim 1, wherein the toripalimab is administered at a dose of about 240 mg once every three weeks.

5. The method of claim 1, wherein the toripalimab is administered at a dose of about 480 mg once every six weeks.

6. A method of treating a cancer in a subject, comprising administering to the subject with an effective amount of an activatable antibody, wherein the activatable antibody comprises an HVR-H1 comprising an amino acid sequence according to a formula YSISSGYHWSWI (SEQ ID NO: 23), an HVR-H2 comprising an amino acid sequence according to a formula LARIDWDDDKYYSTSLKSRL (SEQ ID NO: 35), an HVR-H3 comprising an amino acid sequence according to a formula ARSYVYFDY (SEQ ID NO: 45), an HVR-L1 comprising an amino acid sequence according to the formula RASQSVRGRFLA (SEQ ID NO: 58), an HVR-L2 comprises an amino acid sequence according to a formula DASNRATGI (SEQ ID NO: 66), and an HVR-L3 comprising an amino acid sequence according to a formula YCQQSSSWPPT (SEQ ID NO: 75), and wherein the activatable antibody further comprises: a polypeptide covalently attached to the N-terminus of the light chain of the anti-CTLA4 antibody, said polypeptide comprising, from N-terminus to C-terminus, a masking moiety (MM) and a cleavable moiety (CM), wherein the MM comprises an amino acid sequence EVGSYPNPSSDCVPYYYACAY (SEQ ID NO:192), and the cleavable moiety comprises an amino acid sequence SGRSAGGGGTPLGLAGSGGS (SEQ ID NO:221); and wherein the a activatable antibody is administered at a dose of from about 6 mg/kg to about 20 mg/kg once every three to six weeks.

7. The method of any one of claims 1-6, wherein the cancer is resistant or refractory to a prior therapy, wherein the prior therapy is an inhibitor of CTLA4, PD-1, or a PD-1 ligand.

8. The method of claim 7, wherein the prior therapy is ipilimumab.

9. The method of any one of claims 1-8, wherein the cancer is colorectal cancer (CRC).

10. The method of claim 9, wherein the CRC is microsatellite stable (MSS) CRC.

11. The method of any one of claims 1-8, wherein the cancer is squamous cell carcinoma.

12. The method of any one of claims 1-8, wherein the cancer is anal squamous cell carcinoma or penile squamous cell carcinoma.

13. The method of any one of claims 1-8, wherein the cancer is pancreatic cancer.

14. The method of any one of claim 13, wherein the cancer is pancreatic ductal adenocarcinoma (PDAC).

15. The method of any one of claims 1-8, wherein the cancer is ovarian cancer.

16. The method of any one of claims 1-8, wherein the cancer is NSCLC.

17. The method of any one of claims 1-8, wherein the cancer is hepatocellular carcinoma.

18. The method of any one of claims 1-17, wherein the cancer is an advanced stage metastatic cancer.

19. The method of claim 18, wherein the cancer has metastasized to the lung or the liver.

20. The method of any one of claims 1-18, wherein the activatable anti-CTLA4 antibody comprises a heavy chain variable region comprising the amino acid sequence of SEQ ID NO: 87 or a variant thereof having at least about 90% sequence identity to the amino acid sequence of SEQ ID NO: 87, and a light chain variable region comprising the amino acid sequence of SEQ ID NO: 100 or a variant thereof having at least about 90% sequence identity to the amino acid sequence of SEQ ID NO: 100.

21. The method of claim 20, wherein the heavy chain variable region comprises the amino acid sequence of SEQ ID NO: 87 and the light chain variable region comprises the amino acid sequence of SEQ ID NO: 100.

22. The method of claim 21, wherein the activatable antibody comprises a full heavy chain region of SEQ ID NO:320 or SEQ ID NO:321.

23. The method of claim 22, wherein the activatable antibody comprises a full light chain region of SEQ ID NO:322 or SEQ ID NO:323.

24. The method of any one of claims 1-23, wherein the subject is human.

25. The method of any one of claims 1-24, wherein the activatable anti-CTLA4 antibody and toripalimab are both administered on day 1 of the 3 to 6 week dosing schedule.

26. A method of treating a cancer in a subject, comprising administering to the subject an effective amount of an activatable antibody, wherein the activatable antibody comprises an HVR-H1 comprising an amino acid sequence according to a formula YSISSGYHWSWI (SEQ ID NO: 23), an HVR-H2 comprising an amino acid sequence according to a formula LARIDWDDDKYYSTSLKSRL (SEQ ID NO: 35), an HVR-H3 comprising an amino acid sequence according to a formula ARSYVYFDY (SEQ ID NO: 45), an HVR-L1 comprising an amino acid sequence according to the formula RASQSVRGRFLA (SEQ ID NO: 58), an HVR-L2 comprises an amino acid sequence according to a formula DASNRATGI (SEQ ID NO: 66), and an HVR-L3 comprising an amino acid sequence according to a formula YCQQSSSWPPT (SEQ ID NO: 75), and wherein the activatable antibody further comprises: a polypeptide covalently attached to the N-terminus of the light chain of the anti-CTLA4 antibody, said polypeptide comprising, from N-terminus to C-terminus, a masking moiety (MM) and a cleavable moiety (CM), wherein the MM comprises an amino acid sequence EVGSYPNPSSDCVPYYYACAY (SEQ ID NO:192), and the cleavable moiety comprises an amino acid sequence SGRSAGGGGTPLGLAGSGGS (SEQ ID NO:221), wherein the activatable antibody is administered at a single loading dose of about 20 mg/kg followed by maintenance doses of 10 mg/kg once every three weeks or once every six weeks.

27. The method of claim 26, wherein the first maintenance dose is administered three weeks after administration of the loading dose.

28. The method of claim 26 or claim 27, wherein the maintenance doses are administered once every three weeks.

29. The method of any one of claims 26-28, further comprising administering an effective amount of an anti-PD-1 antibody to the subject.

30. The method of claim 29, wherein the anti-PD-1 antibody is toripalimab.

31. The method of claim 30, wherein the toripalimab is administered at a dose of from about 200 mg to about 400 mg once every three weeks or about 300 mg to about 600 mg once every six weeks.

32. The method of any one of claims 26-31, wherein the cancer is resistant or refractory to a prior therapy, wherein the prior therapy is an inhibitor of CTLA4, PD-1, or a PD-1 ligand.

33. The method of claim 32, wherein the prior therapy is ipilimumab.

34. The method of any one of claims 26-33, wherein the cancer is colorectal cancer (CRC).

35. The method of claim 34, wherein the CRC is microsatellite stable (MSS) CRC.

36. The method of any one of claims 26-33, wherein the cancer is squamous cell carcinoma.

37. The method of any one of claims 26-33, wherein the cancer is anal squamous cell carcinoma or penile squamous cell carcinoma.

38. The method of any one of claims 26-33, wherein the cancer is pancreatic cancer.

39. The method of claim 38, wherein the cancer is pancreatic ductal adenocarcinoma (PDAC).

40. The method of any one of claims 26-33, wherein the cancer is ovarian cancer.

41. The method of any one of claims 26-33, wherein the cancer is NSCLC.

42. The method of any one of claims 26-33, wherein the cancer is hepatocellular carcinoma.

43. The method of any one of claims 26-42, wherein the cancer is an advanced stage metastatic cancer.

44. The method of claim 43, wherein the cancer has metastasized to the lung or the liver.

45. The method of any one of claims 26-44, wherein the activatable antibody comprises a heavy chain variable region comprising the amino acid sequence of SEQ ID NO: 87 or a variant thereof having at least about 90% sequence identity to the amino acid sequence of SEQ ID NO: 87, and a light chain variable region comprising the amino acid sequence of SEQ ID NO: 100 or a variant thereof having at least about 90% sequence identity to the amino acid sequence of SEQ ID NO: 100.

46. The method of claim 45, wherein the heavy chain variable region comprises the amino acid sequence of SEQ ID NO: 87 and the light chain variable region comprises the amino acid sequence of SEQ ID NO: 100.

47. The method of claim 46, wherein the activatable antibody comprises a full heavy chain region of SEQ ID NO:320 or SEQ ID NO:321.

48. The method of claim 47, wherein the activatable antibody comprises a full light chain region of SEQ ID NO:322 or SEQ ID NO:323.

49. The method of any one of claims 26-48, wherein the subject is human.

50. A method of treating a cancer in a subject, comprising administering to the subject an effective amount of an activatable antibody, wherein the activatable antibody comprises an HVR-H1 comprising an amino acid sequence according to a formula YSISSGYHWSWI (SEQ ID NO: 23), an HVR-H2 comprising an amino acid sequence according to a formula LARIDWDDDKYYSTSLKSRL (SEQ ID NO: 35), an HVR-H3 comprising an amino acid sequence according to a formula ARSYVYFDY (SEQ ID NO: 45), an HVR-L1 comprising an amino acid sequence according to the formula RASQSVRGRFLA (SEQ ID NO: 58), an HVR-L2 comprises an amino acid sequence according to a formula DASNRATGI (SEQ ID NO: 66), and an HVR-L3 comprising an amino acid sequence according to a formula YCQQSSSWPPT (SEQ ID NO: 75), and wherein the activatable antibody further comprises: a polypeptide covalently attached to the N-terminus of the light chain of the anti-CTLA4 antibody, said polypeptide comprising, from N-terminus to C-terminus, a masking moiety (MM) and a cleavable moiety (CM), wherein the MM comprises an amino acid sequence EVGSYPNPSSDCVPYYYACAY (SEQ ID NO:192), and the cleavable moiety comprises an amino acid sequence SGRSAGGGGTPLGLAGSGGS (SEQ ID NO:221), wherein the activatable antibody is administered at a dose that provides a steady state concentration of the cleaved antibody of above the EC50 of the cleaved antibody.

51. A method of treating a cancer in a subject, comprising administering to the subject an effective amount of an activatable antibody, wherein the activatable antibody comprises an HVR-H1 comprising an amino acid sequence according to a formula YSISSGYHWSWI (SEQ ID NO: 23), an HVR-H2 comprising an amino acid sequence according to a formula LARIDWDDDKYYSTSLKSRL (SEQ ID NO: 35), an HVR-H3 comprising an amino acid sequence according to a formula ARSYVYFDY (SEQ ID NO: 45), an HVR-L1 comprising an amino acid sequence according to the formula RASQSVRGRFLA (SEQ ID NO: 58), an HVR-L2 comprises an amino acid sequence according to a formula DASNRATGI (SEQ ID NO: 66), and an HVR-L3 comprising an amino acid sequence according to a formula YCQQSSSWPPT (SEQ ID NO: 75), and wherein the activatable antibody further comprises: a polypeptide covalently attached to the N-terminus of the light chain of the anti-CTLA4 antibody, said polypeptide comprising, from N-terminus to C-terminus, a masking moiety (MM) and a cleavable moiety (CM), wherein the MM comprises an amino acid sequence EVGSYPNPSSDCVPYYYACAY (SEQ ID NO:192), and the cleavable moiety comprises an amino acid sequence SGRSAGGGGTPLGLAGSGGS (SEQ ID NO:221), wherein the activatable antibody is administered at a dose that provides a steady state concentration of the cleaved antibody of above the EC90 of the cleaved antibody.

52. A method of treating a cancer in a subject, comprising administering to the subject an effective amount of an activatable antibody, wherein the activatable antibody comprises an HVR-H1 comprising an amino acid sequence according to a formula YSISSGYHWSWI (SEQ ID NO: 23), an HVR-H2 comprising an amino acid sequence according to a formula LARIDWDDDKYYSTSLKSRL (SEQ ID NO: 35), an HVR-H3 comprising an amino acid sequence according to a formula ARSYVYFDY (SEQ ID NO: 45), an HVR-L1 comprising an amino acid sequence according to the formula RASQSVRGRFLA (SEQ ID NO: 58), an HVR-L2 comprises an amino acid sequence according to a formula DASNRATGI (SEQ ID NO: 66), and an HVR-L3 comprising an amino acid sequence according to a formula YCQQSSSWPPT (SEQ ID NO: 75), and wherein the activatable antibody further comprises: a polypeptide covalently attached to the N-terminus of the light chain of the anti-CTLA4 antibody, said polypeptide comprising, from N-terminus to C-terminus, a masking moiety (MM) and a cleavable moiety (CM), wherein the MM comprises an amino acid sequence EVGSYPNPSSDCVPYYYACAY (SEQ ID NO:192), and the cleavable moiety comprises an amino acid sequence SGRSAGGGGTPLGLAGSGGS (SEQ ID NO:221), wherein the activatable antibody is administered at a dose that provides a steady state concentration of the cleaved antibody of from about 100 nM to about 200 nM.

53. The method of claim 52, wherein the activatable antibody is administered at a dose that provides a steady state concentration of the cleaved antibody of from about 100 nM to about 175 nM.

54. The method of claim 52, wherein the activatable antibody is administered at a dose that provides a steady state concentration of the cleaved antibody of from about 100 nM to about 150 nM.

55. The method of claim 52, wherein the activatable antibody is administered at a dose that provides a steady state concentration of the cleaved antibody of from about 150 nM to about 200 nM.

56. The method of any one of claims 50-55, wherein the steady state concentration of the cleaved antibody is measured at the trough level of the anti-CTLA4 antibody.

57. The method of any one of claims 50-56, wherein the activatable antibody is administered as a single loading dose followed by maintenance doses, wherein the amount of the loading dose is higher than the amount of the maintenance doses.

58. The method of claim 57, wherein the loading dose is about 20 mg/kg.

59. The method of claim 57 or claim 58, wherein the maintenance doses are about 10 mg/kg.

60. The method of any one of claims 50-59, further comprising administering an effective amount of an anti-PD-1 antibody to the subject.

61. The method of claim 60, wherein the anti-PD-1 antibody is toripalimab.

62. The method of any one of claims 50-61, wherein the activatable antibody comprises a heavy chain variable region comprising the amino acid sequence of SEQ ID NO: 87 or a variant thereof having at least about 90% sequence identity to the amino acid sequence of SEQ ID NO: 87, and a light chain variable region comprising the amino acid sequence of SEQ ID NO: 100 or a variant thereof having at least about 90% sequence identity to the amino acid sequence of SEQ ID NO: 100.

63. The method of claim 62, wherein the heavy chain variable region comprises the amino acid sequence of SEQ ID NO: 87 and the light chain variable region comprises the amino acid sequence of SEQ ID NO: 100.

64. The method of claim 62, wherein the activatable antibody comprises a full heavy chain region of SEQ ID NO:320 or SEQ ID NO:321.

65. The method of claim 64, wherein the activatable antibody comprises a full light chain region of SEQ ID NO:322 or SEQ ID NO:323.

66. The method of any one of claims 50-65, wherein the subject is human.


SEQUENCE LISTING (328 sequences)

SEQ ID NO: 1 - protein, 11 aa, synthetic construct
  REGION 1..11: note = Synthetic Construct
  VARIANT 1: note = Xaa = Phe or Tyr
  VARIANT 5: note = Xaa = Asp or Gly
  VARIANT 7: note = Xaa = Ala, Gly or Trp
XTFSXYXIHWV

SEQ ID NO: 2 - protein, 12 aa, synthetic construct
  REGION 1..12: note = Synthetic Construct
  VARIANT 4: note = Xaa = Ser or Thr
  VARIANT 7..8: note = Xaa = His or Tyr
  VARIANT 10: note = Xaa = Ala, Asp, or Ser
YSIXSGXXWXWI

SEQ ID NO: 3 - protein, 13 aa, synthetic construct
  REGION 1..13: note = Synthetic Construct
  VARIANT 11: note = Xaa = Gly or Ser
FSLSTGGVAVXWI

SEQ ID NO: 4 - protein, 20 aa, synthetic construct
  REGION 1..20: note = Synthetic Construct
  VARIANT 3: note = Xaa = Asp or Glu
  VARIANT 5: note = Xaa = Ser or Tyr
  VARIANT 14: note = Xaa = Pro or Gln
IGXIXHSGSTYYSXSLKSRV

SEQ ID NO: 5 - protein, 21 aa, synthetic construct
  REGION 1..21: note = Synthetic Construct
  VARIANT 3: note = Xaa = Ile or Trp
  VARIANT 8: note = Xaa = Gly or Ser
  VARIANT 10: note = Xaa = Gly or Ser
  VARIANT 12: note = Xaa = Lys or Asn
IGXISPSXGXTXYAQKFQGRV

SEQ ID NO: 6 - protein, 21 aa, synthetic construct
  REGION 1..21: note = Synthetic Construct
  VARIANT 3: note = Xaa = Ala, Gly, or Ser
  VARIANT 7: note = Xaa = Ser or Tyr
  VARIANT 9: note = Xaa = Gly or Ser
  VARIANT 10: note = Xaa = Ser or Thr
VSXISGXGXXTYYADSVKGRF

SEQ ID NO: 7 - protein, 9 aa, synthetic construct
  REGION 1..9: note = Synthetic Construct
  VARIANT 3: note = Xaa = Gly, Arg, or Ser
  VARIANT 4: note = Xaa = Ala, Ile, or Tyr
  VARIANT 5: note = Xaa = Asp, Val, or Tyr
  VARIANT 6: note = Xaa = Ala, Glu, or Tyr
  VARIANT 9: note = Xaa = Ile or Tyr
ARXXXXFDX

SEQ ID NO: 8 - protein, 10 aa, synthetic construct
  REGION 1..10: note = Synthetic Construct
  VARIANT 3: note = Xaa = Asp or Leu
  VARIANT 5: note = Xaa = Phe or Tyr
  VARIANT 10: note = Xaa = Val or Tyr
ARXGXGYFDX

SEQ ID NO: 9 - protein, 12 aa, synthetic construct
  REGION 1..12: note = Synthetic Construct
  VARIANT 3: note = Xaa = Leu or Arg
  VARIANT 4: note = Xaa = Ile or Pro
  VARIANT 5: note = Xaa = Ala or Tyr
  VARIANT 6: note = Xaa = Ser or Thr
  VARIANT 8: note = Xaa = Thr or Tyr
  VARIANT 9: note = Xaa = Ala or Tyr
ARXXXXAXXFDY

SEQ ID NO: 10 - protein, 17 aa, synthetic construct
  REGION 1..17: note = Synthetic Construct
  VARIANT 4: note = Xaa = Ile or Val
  VARIANT 5: note = Xaa = Ala or His
  VARIANT 6: note = Xaa = Pro or Ser
  VARIANT 13: note = Xaa = Asp or Tyr
  VARIANT 17: note = Xaa = Phe or Val
ARDXXXGSSGYYXGFDX

SEQ ID NO: 11 - protein, 11 aa, synthetic construct
  REGION 1..11: note = Synthetic Construct
  VARIANT 5: note = Xaa = Gly or Ser
  VARIANT 7: note = Xaa = Gly or Ser
  VARIANT 6: note = Xaa = Ile or Val
  VARIANT 9: note = Xaa = Ser or Tyr
  VARIANT 11: note = Xaa = Ala or Asn
RASQXXXSXLX

SEQ ID NO: 12 - protein, 12 aa, synthetic construct
  REGION 1..12: note = Synthetic Construct
  VARIANT 5: note = Xaa = Ser or Thr
  VARIANT 7: note = Xaa = Phe, Arg, or Ser
  VARIANT 8: note = Xaa = Gly or Ser
  VARIANT 10: note = Xaa = Phe or Tyr
RASQXVXXRXLA

SEQ ID NO: 13 - protein, 15 aa, synthetic construct
  REGION 1..15: note = Synthetic Construct
  VARIANT 4: note = Xaa = Glu or Gln
  VARIANT 9: note = Xaa = Asp, Phe, His, or Tyr
  VARIANT 11: note = Xaa = Phe, Ile, or Lys
  VARIANT 15: note = Xaa = Ala, Asp, or His
RASXSVDFXGXSFLX

SEQ ID NO: 14 - skipped

SEQ ID NO: 15 - protein, 11 aa, synthetic construct
  REGION 1..11: note = Synthetic Construct
  VARIANT 3: note = Xaa = Glu, Gln, or Val
  VARIANT 4: note = Xaa = His or Gln
  VARIANT 5: note = Xaa = Ala, Gly, His, Arg, or Ser
  VARIANT 6: note = Xaa = Asp, Leu, Ser, or Tyr
  VARIANT 7: note = Xaa = Glu, Gly, Pro, Gln, or Ser
  VARIANT 8: note = Xaa = Leu, Thr, Val, or Trp
  VARIANT 10: note = Xaa = Phe, Leu, Pro, Trp, or Tyr
YCXXXXXXPXT

SEQ ID NO: 16 - protein, 12 aa, synthetic construct
  REGION 1..12: note = Synthetic Construct
  VARIANT 5: note = Xaa = Ser or Tyr
  VARIANT 6: note = Xaa = Asp or Tyr
  VARIANT 7: note = Xaa = Gln or Tyr
YCQQXXXWPPWT

SEQ ID NO: 17 - protein, 13 aa, synthetic construct
  REGION 1..13: note = Synthetic Construct
  VARIANT 4: note = Xaa = His or Gln
  VARIANT 6: note = Xaa = Thr or Val
  VARIANT 11: note = Xaa = Glu or Val
YCQXYXSSPPXYT

SEQ ID NO: 18 - protein, 11 aa, synthetic construct
  REGION 1..11: note = Synthetic Construct
FTFSDYAIHWV

SEQ ID NO: 19 - protein, 12 aa, synthetic construct
  REGION 1..12: note = Synthetic Construct
YSITSGYYWAWI

SEQ ID NO: 20 - protein, 11 aa, synthetic construct
  REGION 1..11: note = Synthetic Construct
FTFSDYGIHWV

SEQ ID NO: 21 - protein, 12 aa, synthetic construct
  REGION 1..12: note = Synthetic Construct
YSISSGYHWDWI

SEQ ID NO: 22 - protein, 11 aa, synthetic construct
  REGION 1..11: note = Synthetic Construct
FTFSDYWIHWV

SEQ ID NO: 23 - protein, 12 aa, synthetic construct
  REGION 1..12: note = Synthetic Construct
YSISSGYHWSWI

SEQ ID NO: 24 - protein, 13 aa, synthetic construct
  REGION 1..13: note = Synthetic Construct
FSLSTGGVAVSWI

SEQ ID NO: 25 - protein, 13 aa, synthetic construct
  REGION 1..13: note = Synthetic Construct
FSLSTGGVAVGWI

SEQ ID NO: 26 - protein, 11 aa, synthetic construct
  REGION 1..11: note = Synthetic Construct
FTFSGYAIHWV

SEQ ID NO: 27 - protein, 11 aa, synthetic construct
  REGION 1..11: note = Synthetic Construct
YTFSGYGIHWV

SEQ ID NO: 28 - protein, 11 aa, synthetic construct
  REGION 1..11: note = Synthetic Construct
YTFSGYAIHWV

SEQ ID NO: 29 - protein, 12 aa, synthetic construct
  REGION 1..12: note = Synthetic Construct
YSITSGHYWSWI

SEQ ID NO: 30 - protein, 21 aa, synthetic construct
  REGION 1..21: note = Synthetic Construct
IGIISPSSGSTNYAQKFQGRV

SEQ ID NO: 31 - protein, 21 aa, synthetic construct
  REGION 1..21: note = Synthetic Construct
VSSISGSGSTTYYADSVKGRF

SEQ ID NO: 32 - protein, 20 aa, synthetic construct
  REGION 1..20: note = Synthetic Construct
IGEIYHSGSTYYSPSLKSRV

SEQ ID NO: 33 - protein, 21 aa, synthetic construct
  REGION 1..21: note = Synthetic Construct
VSGISGYGGSTYYADSVKGRF

SEQ ID NO: 34 - protein, 21 aa, synthetic construct
  REGION 1..21: note = Synthetic Construct
IGWISPSGGGTKYAQKFQGRV

SEQ ID NO: 35 - protein, 20 aa, synthetic construct
  REGION 1..20: note = Synthetic Construct
LARIDWDDDKYYSTSLKSRL

SEQ ID NO: 36 - protein, 21 aa, synthetic construct
  REGION 1..21: note = Synthetic Construct
VSAISGYGSTTYYADSVKGRF

SEQ ID NO: 37 - protein, 21 aa, synthetic construct
  REGION 1..21: note = Synthetic Construct
IGIISPSGGGTKYAQKFQGRV

SEQ ID NO: 38 - protein, 21 aa, synthetic construct
  REGION 1..21: note = Synthetic Construct
IGIISPSGGSTKYAQKFQGRV

SEQ ID NO: 39 - protein, 20 aa, synthetic construct
  REGION 1..20: note = Synthetic Construct
IGDISHSGSTYYSQSLKSRV

SEQ ID NO: 40 - protein, 17 aa, synthetic construct
  REGION 1..17: note = Synthetic Construct
ARDIHSGSSGYYYGFDV

SEQ ID NO: 41 - protein, 10 aa, synthetic construct
  REGION 1..10: note = Synthetic Construct
ARDGFGYFDY

SEQ ID NO: 42 - protein, 17 aa, synthetic construct
  REGION 1..17: note = Synthetic Construct
ARDVAPGSSGYYDGFDF

SEQ ID NO: 43 - protein, 13 aa, synthetic construct
  REGION 1..13: note = Synthetic Construct
ARHSYYGSGNFDY

SEQ ID NO: 44 - protein, 9 aa, synthetic construct
  REGION 1..9: note = Synthetic Construct
ARGAYEFDY

SEQ ID NO: 45 - protein, 9 aa, synthetic construct
  REGION 1..9: note = Synthetic Construct
ARSYVYFDY

SEQ ID NO: 46 - protein, 12 aa, synthetic construct
  REGION 1..12: note = Synthetic Construct
ARRIATATYFDY

SEQ ID NO: 47 - protein, 12 aa, synthetic construct
  REGION 1..12: note = Synthetic Construct
ARLPYSAYAFDY

SEQ ID NO: 48 - protein, 7 aa, synthetic construct
  REGION 1..7: note = Synthetic Construct
ARHPFAY

SEQ ID NO: 49 - protein, 9 aa, synthetic construct
  REGION 1..9: note = Synthetic Construct
ARRIDAFDI

SEQ ID NO: 50 - protein, 8 aa, synthetic construct
  REGION 1..8: note = Synthetic Construct
ARLYDVAY

SEQ ID NO: 51 - protein, 10 aa, synthetic construct
  REGION 1..10: note = Synthetic Construct
ARLGYGYFDV

SEQ ID NO: 52 - protein, 11 aa, synthetic construct
  REGION 1..11: note = Synthetic Construct
ARGSRTGYFDY

SEQ ID NO: 53 - protein, 15 aa, synthetic construct
  REGION 1..15: note = Synthetic Construct
RASESVDFFGISFLA

SEQ ID NO: 54 - protein, 10 aa, synthetic construct
  REGION 1..10: note = Synthetic Construct
SASSSVSYVY

SEQ ID NO: 55 - protein, 11 aa, synthetic construct
  REGION 1..11: note = Synthetic Construct
RASQGIGSSLA

SEQ ID NO: 56 - protein, 15 aa, synthetic construct
  REGION 1..15: note = Synthetic Construct
RASESVDFFGKSFLH

SEQ ID NO: 57 - protein, 12 aa, synthetic construct
  REGION 1..12: note = Synthetic Construct
RASQSVSSRFLA

SEQ ID NO: 58 - protein, 12 aa, synthetic construct
  REGION 1..12: note = Synthetic Construct
RASQSVRGRFLA

SEQ ID NO: 59 - protein, 12 aa, synthetic construct
  REGION 1..12: note = Synthetic Construct
RASQTVFSRYLA

SEQ ID NO: 60 - protein, 11 aa, synthetic construct
  REGION 1..11: note = Synthetic Construct
RASQGVSSYLA

SEQ ID NO: 61 - protein, 15 aa, synthetic construct
  REGION 1..15: note = Synthetic Construct
RASQSVDFYGISFLD

SEQ ID NO: 62 - protein, 15 aa, synthetic construct
  REGION 1..15: note = Synthetic Construct
RASQSVDFDGFSFLH

SEQ ID NO: 63 - protein, 15 aa, synthetic construct
  REGION 1..15: note = Synthetic Construct
RASQSVDFHGKSFLH

SEQ ID NO: 64 - protein, 15 aa, synthetic construct
  REGION 1..15: note = Synthetic Construct
RASQSVDFYGISFLH

SEQ ID NO: 65 - protein, 11 aa, synthetic construct
  REGION 1..11: note = Synthetic Construct
RASQSISSYLN

SEQ ID NO: 66 - protein, 9 aa, synthetic construct
  REGION 1..9: note = Synthetic Construct
DASNRATGI

SEQ ID NO: 67 - protein, 9 aa, synthetic construct
  REGION 1..9: note = Synthetic Construct
DASSLESGV

SEQ ID NO: 68 - protein, 9 aa, synthetic construct
  REGION 1..9: note = Synthetic Construct
DASNLETGV

SEQ ID NO: 69 - protein, 9 aa, synthetic construct
  REGION 1..9: note = Synthetic Construct
AASTLQSGV

SEQ ID NO: 70 - protein, 13 aa, synthetic construct
  REGION 1..13: note = Synthetic Construct
YCQHYTSSPPVYT

SEQ ID NO: 71 - protein, 11 aa, synthetic construct
  REGION 1..11: note = Synthetic Construct
YCVQGLQTPWT

SEQ ID NO: 72 - protein, 12 aa, synthetic construct
  REGION 1..12: note = Synthetic Construct
YCQQYDQWPPWT

SEQ ID NO: 73 - protein, 11 aa, synthetic construct
  REGION 1..11: note = Synthetic Construct
YCQQSYSWPPT

SEQ ID NO: 74 - protein, 11 aa, synthetic construct
  REGION 1..11: note = Synthetic Construct
YCQQSYPTPLT

SEQ ID NO: 75 - protein, 11 aa, synthetic construct
  REGION 1..11: note = Synthetic Construct
YCQQSSSWPPT

SEQ ID NO: 76 - protein, 12 aa, synthetic construct
  REGION 1..12: note = Synthetic Construct
YCQQSYYWPPWT

SEQ ID NO: 77 - protein, 11 aa, synthetic construct
  REGION 1..11: note = Synthetic Construct
YCQHHYGTPLT

SEQ ID NO: 78 - protein, 13 aa, synthetic construct
  REGION 1..13: note = Synthetic Construct
YCQQYVSSPPEYT

SEQ ID NO: 79 - protein, 11 aa, synthetic construct
  REGION 1..11: note = Synthetic Construct
YCQQRDSWPYT

SEQ ID NO: 80 - protein, 11 aa, synthetic construct
  REGION 1..11: note = Synthetic Construct
YCEQSLEVPFT

SEQ ID NO: 81 - protein, 11 aa, synthetic construct
  REGION 1..11: note = Synthetic Construct
YCVQALQLPLT

SEQ ID NO: 82 - protein, 124 aa, synthetic construct
  REGION 1..124: note = Synthetic Construct
EVQLVESGGGLVQPGGSLRLSCAASGFTFSDYAIHWVRQAPGKGLEWIGIISPSSGSTNYAQKFQGRVTISRDNSKNTLYLQLNSLRAEDTAVYYCARDIHSGSSGYYYGFDVWGQGTLVTVSS

SEQ ID NO: 83 - protein, 118 aa, synthetic construct
  REGION 1..118: note = Synthetic Construct
EVQLVESGGGLVQPGGSLRLSCAASGYSITSGYYWAWIRQAPGKGLEWVSSISGSGSTTYYADSVKGRFTISRDNSKNTLYLQLNSLRAEDTAVYYCARDGFGYFDYWGQGTLVTVSS

SEQ ID NO: 84 - protein, 123 aa, synthetic construct
  REGION 1..123: note = Synthetic Construct
EVQLVESGGGLVQPGGSLRLSCAASGFTFSDYGIHWVRQAPGKGLEWIGEIYHSGSTYYSPSLKSRVTISRDNSKNTLYLQLNSLRAEDTAVYYCARDVAPGSSGYYDGFDFWGQGTLVTVSS

SEQ ID NO: 85 - protein, 121 aa, synthetic construct
  REGION 1..121: note = Synthetic Construct
EVQLVESGGGLVQPGGSLRLSCAASGYSISSGYHWDWIRQAPGKGLEWVSGISGYGGSTYYADSVKGRFTISRDNSKNTLYLQLNSLRAEDTAVYYCARHSYYGSGNFDYWGQGTLVTVSS

SEQ ID NO: 86 - protein, 116 aa, synthetic construct
  REGION 1..116: note = Synthetic Construct
EVQLVESGGGLVQPGGSLRLSCAASGFTFSDYWIHWVRQAPGKGLEWIGWISPSGGGTKYAQKFQGRVTISRDNSKNTLYLQLNSLRAEDTAVYYCARGAYEFDYWGQGTLVTVSS

SEQ ID NO: 87 - protein, 116 aa, synthetic construct
  REGION 1..116: note = Synthetic Construct
EVQLVESGGGLVQPGGSLRLSCAASGYSISSGYHWSWIRQAPGKGLEWLARIDWDDDKYYSTSLKSRLTISRDNSKNTLYLQLNSLRAEDTAVYYCARSYVYFDYWGQGTLVTVSS

SEQ ID NO: 88 - protein, 120 aa, synthetic construct
  REGION 1..120: note = Synthetic Construct
EVQLVESGGGLVQPGGSLRLSCAASGFSLSTGGVAVSWIRQAPGKGLEWIGEIYHSGSTYYSPSLKSRVTISRDNSKNTLYLQLNSLRAEDTAVYYCARRIATATYFDYWGQGTLVTVSS

SEQ ID NO: 89 - protein, 121 aa, synthetic construct
  REGION 1..121: note = Synthetic Construct
EVQLVESGGGLVQPGGSLRLSCAASGFSLSTGGVAVGWIRQAPGKGLEWVSAISGYGSTTYYADSVKGRFTISRDNSKNTLYLQLNSLRAEDTAVYYCARLPYSAYAFDYWGQGTLVTVSS

SEQ ID NO: 90 - protein, 114 aa, synthetic construct
  REGION 1..114: note = Synthetic Construct
EVQLVESGGGLVQPGGSLRLSCAASGFTFSGYAIHWVRQAPGKGLEWIGIISPSGGGTKYAQKFQGRVTISRDNSKNTLYLQLNSLRAEDTAVYYCARHPFAYWGQGTLVTVSS

SEQ ID NO: 91 - protein, 115 aa, synthetic construct
  REGION 1..115: note = Synthetic Construct
EVQLVESGGGLVQPGGSLRLSCAASGYTFSGYGIHWVRQAPGKGLEWIGEIYHSGSTYYSPSLKSRVTISRDNSKNTLYLQLNSLRAEDTAVYYCARRIDAFDIWGQGTLVTVSS

SEQ ID NO: 92 - protein, 115 aa, synthetic construct
  REGION 1..115: note = Synthetic Construct
EVQLVESGGGLVQPGGSLRLSCAASGYTFSGYAIHWVRQAPGKGLEWIGIISPSGGGTKYAQKFQGRVTISRDNSKNTLYLQLNSLRAEDTAVYYCARLYDVAYWGQGTLVTVSS

SEQ ID NO: 93 - protein, 117 aa, synthetic construct
  REGION 1..117: note = Synthetic Construct
EVQLVESGGGLVQPGGSLRLSCAASGFTFSDYAIHWVRQAPGKGLEWIGIISPSGGSTKYAQKFQGRVTISRDNSKNTLYLQLNSLRAEDTAVYYCARLGYGYFDVWGQGTLVTVSS

SEQ ID NO: 94 - protein, 118 aa, synthetic construct
  REGION 1..118: note = Synthetic Construct
EVQLVESGGGLVQPGGSLRLSCAASGYSITSGHYWSWIRQAPGKGLEWIGDISHSGSTYYSQSLKSRVTISRDNSKNTLYLQLNSLRAEDTAVYYCARGSRTGYFDYWGQGTLVTVSS

SEQ ID NO: 95 - protein, 114 aa, synthetic construct
  REGION 1..114: note = Synthetic Construct
DIQLTQSPSSLSASVGDRVTITCRASESVDFFGISFLAWYQQKPGKAPKLLIYDASNRATGIPSRFSGSGSGTDFTLTISSLQPEDFATYYCQHYTSSPPVYTFGQGTKVEIKR

SEQ ID NO: 96 - protein, 107 aa, synthetic construct
  REGION 1..107: note = Synthetic Construct
DIQLTQSPSSLSASVGDRVTITCSASSSVSYVYWYQQKPGKAPKLLIYDASSLESGVPSRFSGSGSGTDFTLTISSLQPEDFATYYCVQGLQTPWTFGQGTKVEIKR

SEQ ID NO: 97 - protein, 109 aa, synthetic construct
  REGION 1..109: note = Synthetic Construct
DIQLTQSPSSLSASVGDRVTITCRASQGIGSSLAWYQQKPGKAPKLLIYDASNRATGIPSRFSGSGSGTDFTLTISSLQPEDFATYYCQQYDQWPPWTFGQGTKVEIKR

SEQ ID NO: 98 - protein, 112 aa, synthetic construct
  REGION 1..112: note = Synthetic Construct
DIQLTQSPSSLSASVGDRVTITCRASESVDFFGKSFLHWYQQKPGKAPKLLIYDASNLETGVPSRFSGSGSGTDFTLTISSLQPEDFATYYCQQSYSWPPTFGQGTKVEIKR

SEQ ID NO: 99 - protein, 109 aa, synthetic construct
  REGION 1..109: note = Synthetic Construct
DIQLTQSPSSLSASVGDRVTITCRASQSVSSRFLAWYQQKPGKAPKLLIYDASNRATGIPSRFSGSGSGTDFTLTISSLQPEDFATYYCQQSYPTPLTFGQGTKVEIKR

SEQ ID NO: 100 - protein, 109 aa, synthetic construct
  REGION 1..109: note = Synthetic Construct
DIQLTQSPSSLSASVGDRVTITCRASQSVRGRFLAWYQQKPGKAPKLLIYDASNRATGIPSRFSGSGSGTDFTLTISSLQPEDFATYYCQQSSSWPPTFGQGTKVEIKR

SEQ ID NO: 101 - protein, 110 aa, synthetic construct
  REGION 1..110: note = Synthetic Construct
DIQLTQSPSSLSASVGDRVTITCRASQTVFSRYLAWYQQKPGKAPKLLIYDASNRATGIPSRFSGSGSGTDFTLTISSLQPEDFATYYCQQSYYWPPWTFGQGTKVEIKR

SEQ ID NO: 102 - protein, 108 aa, synthetic construct
  REGION 1..108: note = Synthetic Construct
DIQLTQSPSSLSASVGDRVTITCRASQGVSSYLAWYQQKPGKAPKLLIYAASTLQSGVPSRFSGSGSGTDFTLTISSLQPEDFATYYCQHHYGTPLTFGQGTKVEIKR

SEQ ID NO: 103 - protein, 114 aa, synthetic construct
  REGION 1..114: note = Synthetic Construct
DIQLTQSPSSLSASVGDRVTITCRASQSVDFYGISFLDWYQQKPGKAPKLLIYDASNRATGIPSRFSGSGSGTDFTLTISSLQPEDFATYYCQQYVSSPPEYTFGQGTKVEIKR

SEQ ID NO: 104 - protein, 112 aa, synthetic construct
  REGION 1..112: note = Synthetic Construct
DIQLTQSPSSLSASVGDRVTITCRASQSVDFDGFSFLHWYQQKPGKAPKLLIYDASSLESGVPSRFSGSGSGTDFTLTISSLQPEDFATYYCQQRDSWPYTFGQGTKVEIKR

SEQ ID NO: 105 - protein, 112 aa, synthetic construct
  REGION 1..112: note = Synthetic Construct
DIQLTQSPSSLSASVGDRVTITCRASQSVDFHGKSFLHWYQQKPGKAPKLLIYDASSLESGVPSRFSGSGSGTDFTLTISSLQPEDFATYYCEQSLEVPFTFGQGTKVEIKR

SEQ ID NO: 106 - protein, 112 aa, synthetic construct
  REGION 1..112: note = Synthetic Construct
DIQLTQSPSSLSASVGDRVTITCRASQSVDFYGISFLHWYQQKPGKAPKLLIYDASSLESGVPSRFSGSGSGTDFTLTISSLQPEDFATYYCVQALQLPLTFGQGTKVEIKR

SEQ ID NO: 107 - protein, 108 aa, synthetic construct
  REGION 1..108: note = Synthetic Construct
DIQLTQSPSSLSASVGDRVTITCRASQSISSYLNWYQQKPGKAPKLLIYDASNLETGVPSRFSGSGSGTDFTLTISSLQPEDFATYYCQHHYGTPLTFGQGTKVEIKR

SEQ ID NO: 108 - DNA, 372 bp, synthetic construct
  misc_feature 1..372: note = Synthetic Construct
  source 1..372: mol_type = other DNA
gaggttcagctggtggagtctggcggtggcctggtgcagccagggggctcactccgtttgtcctgtgcagcttccggattcaccttctccgactacgctattcactgggtgcgtcaggccccgggtaagggactcgagtggatcggtatcatctccccatctagcggttctactaactacgcccagaagttccagggtcgtgtgactataagtcgcgacaattcgaaaaacacactgtacctacaactgaacagcttaagagctgaggacactgccgtctattattgcgccagagacattcactctggttcttctggttactactacggtttcgacgtctggggtcaaggaacactagtcaccgtctcctcg

SEQ ID NO: 109 - DNA, 354 bp, synthetic construct
  misc_feature 1..354: note = Synthetic Construct
  source 1..354: mol_type = other DNA
gaggttcagctggtggagtctggcggtggcctggtgcagccagggggctcactccgtttgtcctgtgcagcttccggatactctatcacctctggttactactgggcctggattcgtcaggccccgggtaagggcctcgagtgggtgtcttccatctctggttccggttctactacctactacgccgactctgtcaagggccgtttcactataagtcgcgacaattcgaaaaacacactgtacctacaactgaacagcttaagagctgaggacactgccgtctattattgcgccagagatggtttcggctacttcgactactggggtcaaggaacactagtcaccgtctcctcg

SEQ ID NO: 110 - DNA, 369 bp, synthetic construct
  misc_feature 1..369: note = Synthetic Construct
  source 1..369: mol_type = other DNA
gaggttcagctggtggagtctggcggtggcctggtgcagccagggggctcactccgtttgtcctgtgcagcttccggattcaccttctccgactacggtattcactgggtgcgtcaggccccgggtaagggcctcgagtggatcggtgaaatctaccactctggttctacctactactctccatctctgaagtctcgtgtgactataagtcgcgacaattcgaaaaacacactgtacctacaactgaacagcttaagagctgaggacactgccgtctattattgcgccagagacgttgcccctggttcttctggttactacgacggtttcgacttctggggtcaaggaacactagtcaccgtctcctcg

SEQ ID NO: 111 - DNA, 363 bp, synthetic construct
  misc_feature 1..363: note = Synthetic Construct
  source 1..363: mol_type = other DNA
gaggttcagctggtggagtctggcggtggcctggtgcagccagggggctcactccgtttgtcctgtgcagcttccggatactctatctcctctggttaccactgggactggattcgtcaggccccgggtaagggcctcgagtgggtgtctggtatctctggttacggtggttctacctactacgccgactctgtcaagggccgtttcactataagtcgcgacaattcgaaaaacacactgtacctacaactgaacagcttaagagctgaggacactgccgtctattattgcgccagacacagttattacggttccggtaatttcgactactggggtcaaggaacactagtcaccgtctcctcg

SEQ ID NO: 112 - DNA, 348 bp, synthetic construct
  misc_feature 1..348: note = Synthetic Construct
  source 1..348: mol_type = other DNA
gaggttcagctggtggagtctggcggtggcctggtgcagccagggggctcactccgtttgtcctgtgcagcttccggattcaccttctccgactactggattcactgggtgcgtcaggccccgggtaagggcctcgagtggatcggttggatctccccatctggcggtggtactaagtacgcccagaagttccagggtcgtgtgactataagtcgcgacaattcgaaaaacacactgtacctacaactgaacagcttaagagctgaggacactgccgtctattattgcgccagaggggcttacgaatttgactactggggtcaaggaacactagtcaccgtctcctcg

SEQ ID NO: 113 - DNA, 348 bp, synthetic construct
  misc_feature 1..348: note = Synthetic Construct
  source 1..348: mol_type = other DNA
gaggttcagctggtggagtctggcggtggcctggtgcagccagggggctcactccgtttgtcctgtgcagcttccggatactctatctcctctggttaccactggagctggattcgtcaggccccgggtaagggcctcgagtggctggcccggatcgactgggacgatgacaagtactactctacctctctgaagtctcgtctgactataagtcgcgacaattcgaaaaacacactgtacctacaactgaacagcttaagagctgaggacactgccgtctattattgcgccagatcgtacgtgtacttcgactactggggtcaaggaacactagtcaccgtctcctcg

SEQ ID NO: 114 - DNA, 360 bp, synthetic construct
  misc_feature 1..360: note = Synthetic Construct
  source 1..360: mol_type = other DNA
gaggttcagctggtggagtctggcggtggcctggtgcagccagggggctcactccgtttgtcctgtgcagcttccggattctctctgtctaccggcggtgtggctgtgagctggattcgtcaggccccgggtaagggcctcgagtggatcggtgaaatctaccactctggttctacctactactctccatctctgaagtctcgtgtgactataagtcgcgacaattcgaaaaacacactgtacctacaactgaacagcttaagagctgaggacactgccgtctattattgcgcccgtcgtatcgccaccgctacttacttcgactactggggtcaaggaacactagtcaccgtctcctcg

SEQ ID NO: 115 - DNA, 363 bp, synthetic construct
  misc_feature 1..363: note = Synthetic Construct
  source 1..363: mol_type = other DNA
gaggttcagctggtggagtctggcggtggcctggtgcagccagggggctcactccgtttgtcctgtgcagcttccggattctctctgtctaccggcggtgtggctgtgggctggattcgtcaggccccgggtaagggcctcgagtgggtgtctgctatctctggttacggttctactacctactacgccgactctgtcaagggccgtttcactataagtcgcgacaattcgaaaaacacactgtacctacaactgaacagcttaagagctgaggacactgccgtctattattgcgccagattgccatactccgcctacgctttcgactactggggtcaaggaacactagtcaccgtctcctcg

SEQ ID NO: 116 - DNA, 342 bp, synthetic construct
  misc_feature 1..342: note = Synthetic Construct
  source 1..342: mol_type = other DNA
gaggttcagctggtggagtctggcggtggcctggtgcagccagggggctcactccgtttgtcctgtgcagcttccggattcaccttctccggctacgctattcactgggtgcgtcaggccccgggtaagggcctcgagtggatcggtatcatctccccatctggcggtggtactaagtacgcccagaagttccagggtcgtgtgactataagtcgcgacaattcgaaaaacacactgtacctacaactgaacagcttaagagctgaggacactgccgtctattattgcgccagacacccattcgcctactggggtcaaggaacactagtcaccgtctcctcg

SEQ ID NO: 117 - DNA, 345 bp, synthetic construct
  misc_feature 1..345: note = Synthetic Construct
  source 1..345: mol_type = other DNA
gaggttcagctggtggagtctggcggtggcctggtgcagccagggggctcactccgtttgtcctgtgcagcttccggatacaccttctccggctacggtattcactgggtgcgtcaggccccgggtaagggcctcgagtggatcggtgaaatctaccactctggttctacctactactctccatctctgaagtctcgtgtgactataagtcgcgacaattcgaaaaacacactgtacctacaactgaacagcttaagagctgaggacactgccgtctattattgcgccagaagaattgacgccttcgacatctggggtcaaggaacactagtcaccgtctcctcg

SEQ ID NO: 118 - DNA, 345 bp, synthetic construct
  misc_feature 1..345: note = Synthetic Construct
  source 1..345: mol_type = other DNA
gaggttcagctggtggagtctggcggtggcctggtgcagccagggggctcactccgtttgtcctgtgcagcttccggatacaccttctccggctacgctattcactgggtgcgtcaggccccgggtaagggcctcgagtggatcggtatcatctccccatctggcggtggtactaagtacgcccagaagttccagggtcgtgtgactataagtcgcgacaattcgaaaaacacactgtacctacaactgaacagcttaagagctgaggacactgccgtctattattgcgccagactctatgacgttgcctactggggtcaaggaacactagtcaccgtctcctcg

SEQ ID NO: 119 - DNA, 351 bp, synthetic construct
  misc_feature 1..351: note = Synthetic Construct
  source 1..351: mol_type = other DNA
gaggttcagctggtggagtctggcggtggcctggtgcagccagggggctcactccgtttgtcctgtgcagcttccggattcaccttctccgactacgctattcactgggtgcgtcaggccccgggtaagggcctcgagtggatcggtatcatctccccatctggcggttctactaagtacgcccagaagttccagggtcgtgtgactataagtcgcgacaattcgaaaaacacactgtacctacaactgaacagcttaagagctgaggacactgccgtctattattgcgccagactcggttacgggtacttcgacgtctggggtcaaggaacactagtcaccgtctcctcg

SEQ ID NO: 120 - DNA, 354 bp, synthetic construct
  misc_feature 1..354: note = Synthetic Construct
  source 1..354: mol_type = other DNA
gaggttcagctggtggagtctggcggtggcctggtgcagccagggggctcactccgtttgtcctgtgcagcttccggatactctatcacctctggtcactactggagctggattcgtcaggccccgggtaagggcctcgagtggatcggtgacatctcccactctggttctacctactactctcaatctctgaagtctcgtgtgactataagtcgcgacaattcgaaaaacacactgtacctacaactgaacagcttaagagctgaggacactgccgtctattattgcgcgcgtggtagtaggaccggctacttcgactattggggtcaaggaacactagtcaccgtctcctcg

SEQ ID NO: 121 - DNA, 342 bp, synthetic construct
  misc_feature 1..342: note = Synthetic Construct
  source 1..342: mol_type = other DNA
gatatccagttgacccagtccccgagttccctgtccgcctctgtgggcgatcgggtcaccatcacctgccgtgcctctgagtctgtggacttcttcggtatctctttcctggcctggtatcaacagaaaccaggaaaagctccgaagcttctgatctacgacgcctctaaccgtgccaccggtatcccatctcgcttctctggatccggttccgggacggatttcactctgaccatcagcagtctgcagccggaagacttcgcaacttattactgccagcactacacctcttcgccaccagtgtacaccttcggacagggtaccaaggtggagatcaaacga

SEQ ID NO: 122 - DNA, 321 bp, synthetic construct
  misc_feature 1..321: note = Synthetic Construct
  source 1..321: mol_type = other DNA
gatatccagttgacccagtccccgagttccctgtccgcctctgtgggcgatcgggtcaccatcacctgctctgcctcttctagcgtgagctacgtgtactggtatcaacagaaaccaggaaaagctccgaagcttctgatctacgacgcctcttctctggaatctggtgtgccatctcgcttctctggatccggttccgggacggatttcactctgaccatcagcagtctgcagccggaagacttcgcaacttattactgcgtgcagggtcttcagaccccttggaccttcggacagggtaccaaggtggagatcaaacga

SEQ ID NO: 123 - DNA, 327 bp, synthetic construct
  misc_feature 1..327: note = Synthetic Construct
  source 1..327: mol_type = other DNA
gatatccagttgacccagtccccgagttccctgtccgcctctgtgggcgatcgggtcaccatcacctgccgtgcctctcagggtattggctcttccctggcttggtatcaacagaaaccaggaaaagctccgaagcttctgatctacgacgcctctaaccgtgccaccggtatcccatctcgcttctctggatccggttccgggacggatttcactctgaccatcagcagtctgcagccggaagacttcgcaacttattactgccagcagtacgaccaatggccaccttggaccttcggacagggtaccaaggtggagatcaaacga

SEQ ID NO: 124 - DNA, 336 bp, synthetic construct
  misc_feature 1..336: note = Synthetic Construct
  source 1..336: mol_type = other DNA
gatatccagttgacccagtccccgagttccctgtccgcctctgtgggcgatcgggtcaccatcacctgccgtgcctctgagtctgtggacttcttcggtaagtctttcctgcactggtatcaacagaaaccaggaaaagctccgaagcttctgatctacgacgcctctaacctggaaaccggtgtgccatctcgcttctctggatccggttccgggacggatttcactctgaccatcagcagtctgcagccggaagacttcgcaacttattactgccagcagtcctactcctggcctccgaccttcggacagggtaccaaggtggagatcaaacga

SEQ ID NO: 125 - DNA, 327 bp, synthetic construct
  misc_feature 1..327: note = Synthetic Construct
  source 1..327: mol_type = other DNA
gatatccagttgacccagtccccgagttccctgtccgcctctgtgggcgatcgggtcaccatcacctgccgtgcctctcagtctgtgagcagccgtttcctggcctggtatcaacagaaaccaggaaaagctccgaagcttctgatctacgacgcctctaaccgtgccaccggtatcccatctcgcttctctggatccggttccgggacggatttcactctgaccatcagcagtctgcagccggaagacttcgcaacttattactgccagcagtcctaccccacccctcttaccttcggacagggtaccaaggtggagatcaaacga

SEQ ID NO: 126 - DNA, 327 bp, synthetic construct
  misc_feature 1..327: note = Synthetic Construct
  source 1..327: mol_type = other DNA
gatatccagttgacccagtccccgagttccctgtccgcctctgtgggcgatcgggtcaccatcacctgccgtgcctctcagtctgtgcgcggccgtttcctggcctggtatcaacagaaaccaggaaaagctccgaagcttctgatctacgacgcctctaaccgtgccaccggtatcccatctcgcttctctggatccggttccgggacggatttcactctgaccatcagcagtctgcagccggaagacttcgcaacttattactgccagcagtcctcctcctggcctccgaccttcggacagggtaccaaggtggagatcaaacga

SEQ ID NO: 127 - DNA, 330 bp, synthetic construct
  misc_feature 1..330: note = Synthetic Construct
  source 1..330: mol_type = other DNA
gatatccagttgacccagtccccgagttccctgtccgcctctgtgggcgatcgggtcaccatcacctgccgtgcctctcagaccgtgttctctcgttacctggcttggtatcaacagaaaccaggaaaagctccgaagcttctgatctacgacgcctctaaccgtgccaccggtatcccatctcgcttctctggatccggttccgggacggatttcactctgaccatcagcagtctgcagccggaagacttcgcaacttattactgccagcagtcctactactggccaccttggaccttcggacagggtaccaaggtggagatcaaacga

SEQ ID NO: 128 - DNA, 324 bp, synthetic construct
  misc_feature 1..324: note = Synthetic Construct
  source 1..324: mol_type = other DNA
gatatccagttgacccagtccccgagttccctgtccgcctctgtgggcgatcgggtcaccatcacctgccgtgcctctcagggtgtgtcttcttacctggcctggtatcaacagaaaccaggaaaagctccgaagcttctgatctacgccgcctctaccttgcagtctggtgtgccatctcgcttctctggatccggttccgggacggatttcactctgaccatcagcagtctgcagccggaagacttcgcaacttactactgccagcaccactacggcaccccactgaccttcggtcagggtaccaaggtggagatcaaacga

SEQ ID NO: 129 - DNA, 342 bp, synthetic construct
  misc_feature 1..342: note = Synthetic Construct
  source 1..342: mol_type = other DNA
gatatccagttgacccagtccccgagttccctgtccgcctctgtgggcgatcgggtcaccatcacctgccgtgcctctcagtctgtggacttctacggtatctctttcctggactggtatcaacagaaaccaggaaaagctccgaagcttctgatctacgacgcctctaaccgtgccaccggtatcccatctcgcttctctggatccggttccgggacggatttcactctgaccatcagcagtctgcagccggaagacttcgcaacttattactgccagcagtacgtctcttcgccaccagagtacaccttcggacagggtaccaaggtggagatcaaacga

SEQ ID NO: 130 - DNA, 336 bp, synthetic construct
  misc_feature 1..336: note = Synthetic Construct
  source 1..336: mol_type = other DNA
gatatccagttgacccagtccccgagttccctgtccgcctctgtgggcgatcgggtcaccatcacctgccgtgcctctcagtctgtggacttcgacggtttctctttcctgcactggtatcaacagaaaccaggaaaagctccgaagcttctgatctacgacgcctcttctctggaatctggtgtgccatctcgcttctctggatccggttccgggacggatttcactctgaccatcagcagtctgcagccggaagacttcgcaacttattactgccagcagcgtgactcctggccttacaccttcggacagggtaccaaggtggagatcaaacga

SEQ ID NO: 131 - DNA, 336 bp, synthetic construct
  misc_feature 1..336: note = Synthetic Construct
  source 1..336: mol_type = other DNA
gatatccagttgacccagtccccgagttccctgtccgcctctgtgggcgatcgggtcaccatcacctgccgtgcctctcagtctgtggacttccacggtaagtctttcctgcactggtatcaacagaaaccaggaaaagctccgaagcttctgatctacgacgcctcttctctggaatctggtgtgccatctcgcttctctggatccggttccgggacggatttcactctgaccatcagcagtctgcagccggaagacttcgcaacttattactgcgagcaatccctggaagtcccattcaccttcggacagggtaccaaggtggagatcaaacga

SEQ ID NO: 132 - DNA, 336 bp, synthetic construct
  misc_feature 1..336: note = Synthetic Construct
  source 1..336: mol_type = other DNA
gatatccagttgacccagtccccgagttccctgtccgcctctgtgggcgatcgggtcaccatcacctgccgtgcctctcagtctgtggacttctacggtatctctttcctgcactggtatcaacagaaaccaggaaaagctccgaagcttctgatctacgacgcctcttctctggaatctggtgtgccatctcgcttctctggatccggttccgggacggatttcactctgaccatcagcagtctgcagccggaagacttcgcaacttattactgcgtgcaggctcttcagttgcctcttaccttcggacagggtaccaaggtggagatcaaacga

SEQ ID NO: 133 - DNA, 324 bp, synthetic construct
  misc_feature 1..324: note = Synthetic Construct
  source 1..324: mol_type = other DNA
gatatccagttgacccagtccccgagttccctgtccgcctctgtgggcgatcgggtcaccatcacctgccgtgcctctcagtctatctcttcttacctgaactggtatcaacagaaaccaggaaaagctccgaagcttctgatctacgacgcctctaacctggaaaccggtgtgccatctcgcttctctggatccggttccgggacggatttcactctgaccatcagcagtctgcagccggaagacttcgcaacttactactgccagcaccactacggcaccccactgaccttcggtcagggtaccaaggtggagatcaaacga

SEQ ID NO: 134-140 - skipped (7 entries)

SEQ ID NO: 141 - protein, 16 aa, synthetic construct
  REGION 1..16: note = Synthetic Construct
NFVADSCPDHPYPCSA

SEQ ID NO: 142 - protein, 16 aa, synthetic construct
  REGION 1..16: note = Synthetic Construct
IVHHSDCDAFYPYCDS

SEQ ID NO: 143 - protein, 16 aa, synthetic construct
  REGION 1..16: note = Synthetic Construct
YSAYPACDSHYPYCNS

SEQ ID NO: 144 - protein, 16 aa, synthetic construct
  REGION 1..16: note = Synthetic Construct
PNPSSDCVPYYYACAY

SEQ ID NO: 145 - protein, 16 aa, synthetic construct
  REGION 1..16: note = Synthetic Construct
YSAYPACDSHYPYCQS

SEQ ID NO: 146 - protein, 16 aa, synthetic construct
  REGION 1..16: note = Synthetic Construct
PQPSSDCVPYYYACAY

SEQ ID NO: 147 - protein, 16 aa, synthetic construct
  REGION 1..16: note = Synthetic Construct
PNPASDCVPYYYACAY

SEQ ID NO: 148 - protein, 5 aa, synthetic construct
  REGION 1..5: note = Synthetic Construct
EVGSY

SEQ ID NO: 149 - protein, 5 aa, synthetic construct
  REGION 1..5: note = Synthetic Construct
SGRSA

SEQ ID NO: 150 - protein, 6 aa, synthetic construct
  REGION 1..6: note = Synthetic Construct
PLGLAG

SEQ ID NO: 151 - protein, 7 aa, synthetic construct
  REGION 1..7: note = Synthetic Construct
ENLYFQG

SEQ ID NO: 152 - protein, 26 aa, synthetic construct
  REGION 1..26: note = Synthetic Construct
  VARIANT 6..11: note = Xaa = Asp, Ala, Tyr, Ser, Thr, Asn, Ile, Leu, Phe, Val, His, or Pro
  VARIANT 13..18: note = Xaa = Asp, Ala, Tyr, Ser, Thr, Asn, Ile, Leu, Phe, Val, His, or Pro
  VARIANT 20..21: note = Xaa = Asp, Ala, Tyr, Ser, Thr, Asn, Ile, Leu, Phe, Val, His, or Pro
EVGSYXXXXXXCXXXXXXCXXSGRSA

SEQ ID NO: 153 - protein, 26 aa, synthetic construct
  REGION 1..26: note = Synthetic Construct
  VARIANT 6..11: note = Xaa = Asp, Ala, Tyr, Ser, Thr, Asn, Ile, Leu, Phe, Val, His, or Pro
  VARIANT 20..21: note = Xaa = Asp, Ala, Tyr, Ser, Thr, Asn, Ile, Leu, Phe, Val, His, or Pro
  VARIANT 13..18: note = Xaa = Ala, Cys, Asp, Glu, Phe, Gly, His, Ile, Lys, Leu, Met, Asn, Pro, Gln, Arg, Ser, Thr, Val, Trp, or Tyr
EVGSYXXXXXXCXXXXXXCXXSGRSA

SEQ ID NO: 154 - protein, 28 aa, synthetic construct
  REGION 1..28: note = Synthetic Construct
  VARIANT 6..11: note = Xaa = Asp, Ala, Tyr, Ser, Thr, Asn, Ile, Leu, Phe, Val, His, or Pro
  VARIANT 13..20: note = Xaa = Asp, Ala, Tyr, Ser, Thr, Asn, Ile, Leu, Phe, Val, His, or Pro
  VARIANT 22..23: note = Xaa = Asp, Ala, Tyr, Ser, Thr, Asn, Ile, Leu, Phe, Val, His, or Pro
EVGSYXXXXXXCXXXXXXXXCXXSGRSA

SEQ ID NO: 155 - protein, 28 aa, synthetic construct
  REGION 1..28: note = Synthetic Construct
  VARIANT 6..11: note = Xaa = Asp, Ala, Tyr, Ser, Thr, Asn, Ile, Leu, Phe, Val, His, or Pro
  VARIANT 22..23: note = Xaa = Asp, Ala, Tyr, Ser, Thr, Asn, Ile, Leu, Phe, Val, His, or Pro
  VARIANT 13..20: note = Xaa = Ala, Cys, Asp, Glu, Phe, Gly, His, Ile, Lys, Leu, Met, Asn, Pro, Gln, Arg, Ser, Thr, Val, Trp, or Tyr
EVGSYXXXXXXCXXXXXXXXCXXSGRSA

SEQ ID NO: 156 - protein, 5 aa, synthetic construct
  REGION 1..5: note = Synthetic Construct
GGGGS

SEQ ID NO: 157 - protein, 4 aa, synthetic construct
  REGION 1..4: note = Synthetic Construct
SGGS

SEQ ID NO: 158 - protein, 4 aa, synthetic construct
  REGION 1..4: note = Synthetic Construct
GGSG

SEQ ID NO: 159 - protein, 5 aa, synthetic construct
  REGION 1..5: note = Synthetic Construct
GGSGG

SEQ ID NO: 160 - protein, 5 aa, synthetic construct
  REGION 1..5: note = Synthetic Construct
GSGSG

SEQ ID NO: 161 - protein, 5 aa, synthetic construct
  REGION 1..5: note = Synthetic Construct
GSGGG

SEQ ID NO: 162 - protein, 5 aa, synthetic construct
  REGION 1..5: note = Synthetic Construct
GGGSG

SEQ ID NO: 163 - protein, 5 aa, synthetic construct
  REGION 1..5: note = Synthetic Construct
GSSSG

SEQ ID NO: 164 - protein, 42 aa, synthetic construct
  REGION 1..42: note = Synthetic Construct
  VARIANT 6: note = Xaa = Ala, Asp, Ile, Asn, Pro, or Tyr
  VARIANT 7: note = Xaa = Ala, Phe, Asn, Ser, or Val
  VARIANT 8: note = Xaa = Ala, His, Leu, Pro, Ser, Val, or Tyr
  VARIANT 9: note = Xaa = Ala, His, Ser, or Tyr
  VARIANT 10: note = Xaa = Ala, Asp, Pro, Ser, Val, or Tyr
  VARIANT 11: note = Xaa = Ala, Asp, Leu, Ser, or Tyr
  VARIANT 13: note = Xaa = Asp, Pro, or Val
  VARIANT 14: note = Xaa = Ala, Asp, His, Pro, Ser, or Thr
  VARIANT 15: note = Xaa = Ala, Asp, Phe, His, Pro, or Tyr
  VARIANT 16: note = Xaa = Leu, Pro, or Tyr
  VARIANT 17: note = Xaa = Phe, Pro, or Tyr
  VARIANT 18: note = Xaa = Ala, Pro, Ser, or Tyr
  VARIANT 20: note = Xaa = Ala, Asp, Asn, Ser, Thr, or Tyr
  VARIANT 21: note = Xaa = Ala, Ser, or Tyr
EVGSYXXXXXXCXXXXXXCXXSGRSAGGGGTENLYFQGSGGS

SEQ ID NO: 165 - protein, 42 aa, synthetic construct
  REGION 1..42: note = Synthetic Construct
EVGSYDALHYACPPDYYACYYSGRSAGGGGTENLYFQGSGGS

SEQ ID NO: 166 - protein, 42 aa, synthetic construct
  REGION 1..42: note = Synthetic Construct
EVGSYNSYHAYCPHPLYPCTASGRSAGGGGTENLYFQGSGGS

SEQ ID NO: 167 - protein, 42 aa, synthetic construct
  REGION 1..42: note = Synthetic Construct
EVGSYASSAVLCVTAYFSCNSSGRSAGGGGTENLYFQGSGGS

SEQ ID NO: 168 - protein, 41 aa, synthetic construct
  REGION 1..41: note = Synthetic Construct
EVGSYNFVADSCPDHPYPCSASGRSAGGGGSPLGLAGSGGS

SEQ ID NO: 169 - protein, 42 aa, synthetic construct
  REGION 1..42: note = Synthetic Construct
EVGSYNFVADSCPDHPYPCSASGRSAGGGGTENLYFQGSGGS

SEQ ID NO: 170 - protein, 41 aa, synthetic construct
  REGION 1..41: note = Synthetic Construct
EVGSYIVHHSDCDAFYPYCDSSGRSAGGGGSPLGLAGSGGS

SEQ ID NO: 171 - protein, 42 aa, synthetic construct
  REGION 1..42: note = Synthetic Construct
EVGSYIVHHSDCDAFYPYCDSSGRSAGGGGTENLYFQGSGGS

SEQ ID NO: 172 - protein, 41 aa, synthetic construct
  REGION 1..41: note = Synthetic Construct
EVGSYYSAYPACDSHYPYCNSSGRSAGGGGSPLGLAGSGGS

SEQ ID NO: 173 - protein, 42 aa, synthetic construct
  REGION 1..42: note = Synthetic Construct
EVGSYYSAYPACDSHYPYCNSSGRSAGGGGTENLYFQGSGGS

SEQ ID NO: 174 - protein, 41 aa, synthetic construct
  REGION 1..41: note = Synthetic Construct
EVGSYPNPSSDCVPYYYACAYSGRSAGGGGSPLGLAGSGGS

SEQ ID NO: 175 - protein, 42 aa, synthetic construct
  REGION 1..42: note = Synthetic Construct
EVGSYPNPSSDCVPYYYACAYSGRSAGGGGTENLYFQGSGGS

SEQ ID NO: 176 - protein, 41 aa, synthetic construct
  REGION 1..41: note = Synthetic Construct
EVGSYYSAYPACDSHYPYCQSSGRSAGGGGSPLGLAGSGGS

SEQ ID NO: 177 - protein, 41 aa, synthetic construct
  REGION 1..41: note = Synthetic Construct
EVGSYYSAYPACDSHYPYCNSAGRSAGGGGSPLGLAGSGGS

SEQ ID NO: 178 - protein, 41 aa, synthetic construct
  REGION 1..41: note = Synthetic Construct
EVGSYPQPSSDCVPYYYACAYSGRSAGGGGSPLGLAGSGGS

SEQ ID NO: 179 - protein, 41 aa, synthetic construct
  REGION 1..41: note = Synthetic Construct
EVGSYPNPASDCVPYYYACAYSGRSAGGGGSPLGLAGSGGS

SEQ ID NO: 180 - protein, 20 aa, synthetic construct
  REGION 1..20: note = Synthetic Construct
SGRSAGGGGSPLGLAGSGGS

SEQ ID NO: 181 - protein, 11 aa, synthetic construct
  REGION 1..11: note = Synthetic Construct
  VARIANT 1: note = Present in repeats of at least two and up to ten
  VARIANT 1: note = Xaa = Ala, Cys, Asp, Glu, Phe, Gly, His, Ile, Lys, Leu, Met, Asn, Pro, Gln, Arg, Ser, Thr, Val, Trp, or Tyr
  VARIANT 10..11: note = Xaa = Ala, Cys, Asp, Glu, Phe, Gly, His, Ile, Lys, Leu, Met, Asn, Pro, Gln, Arg, Ser, Thr, Val, Trp, or Tyr
XCPDHPYPCXX

SEQ ID NO: 182 - protein, 11 aa, synthetic construct
  REGION 1..11: note = Synthetic Construct
  VARIANT 1: note = Present in repeats of at least two and up to ten
  VARIANT 1: note = Xaa = Ala, Cys, Asp, Glu, Phe, Gly, His, Ile, Lys, Leu, Met, Asn, Pro, Gln, Arg, Ser, Thr, Val, Trp, or Tyr
  VARIANT 10..11: note = Xaa = Ala, Cys, Asp, Glu, Phe, Gly, His, Ile, Lys, Leu, Met, Asn, Pro, Gln, Arg, Ser, Thr, Val, Trp, or Tyr
XCDAFYPYCXX

SEQ ID NO: 183 - protein, 11 aa, synthetic construct
  REGION 1..11: note = Synthetic Construct
  VARIANT 1: note = Present in repeats of at least two and up to ten
  VARIANT 1: note = Xaa = Ala, Cys, Asp, Glu, Phe, Gly, His, Ile, Lys, Leu, Met, Asn, Pro, Gln, Arg, Ser, Thr, Val, Trp, or Tyr
  VARIANT 10..11: note = Xaa = Ala, Cys, Asp, Glu, Phe, Gly, His, Ile, Lys, Leu, Met, Asn, Pro, Gln, Arg, Ser, Thr, Val, Trp, or Tyr
XCDSHYPYCXX

SEQ ID NO: 184 - protein, 11 aa, synthetic construct
  REGION 1..11: note = Synthetic Construct
  VARIANT 1: note = Present in repeats of at least two and up to ten
  VARIANT 1: note = Xaa = Ala, Cys, Asp, Glu, Phe, Gly, His, Ile, Lys, Leu, Met, Asn, Pro, Gln, Arg, Ser, Thr, Val, Trp, or Tyr
  VARIANT 10..11: note = Xaa = Ala, Cys, Asp, Glu, Phe, Gly, His, Ile, Lys, Leu, Met, Asn, Pro, Gln, Arg, Ser, Thr, Val, Trp, or Tyr
XCVPYYYACXX

SEQ ID NO: 185-188 - skipped (4 entries)

SEQ ID NO: 189 - protein, 21 aa, synthetic construct
  REGION 1..21: note = Synthetic Construct
EVGSYNFVADSCPDHPYPCSA

SEQ ID NO: 190 - protein, 21 aa, synthetic construct
  REGION 1..21: note = Synthetic Construct
EVGSYIVHHSDCDAFYPYCDS

SEQ ID NO: 191 - protein, 21 aa, synthetic construct
  REGION 1..21: note = Synthetic Construct
EVGSYYSAYPACDSHYPYCNS

SEQ ID NO: 192 - protein, 21 aa, synthetic construct
  REGION 1..21: note = Synthetic Construct
EVGSYPNPSSDCVPYYYACAY

SEQ ID NO: 193 - protein, 21 aa, synthetic construct
  REGION 1..21: note = Synthetic Construct
EVGSYYSAYPACDSHYPYCQS

SEQ ID NO: 194 - skipped

SEQ ID NO: 195 - protein, 21 aa, synthetic construct
  REGION 1..21: note = Synthetic Construct
EVGSYPQPSSDCVPYYYACAY

SEQ ID NO: 196 - protein, 21 aa, synthetic construct
  REGION 1..21: note = Synthetic Construct
EVGSYPNPASDCVPYYYACAY

SEQ ID NO: 197 - protein, 41 aa, synthetic construct
  REGION 1..41: note = Synthetic Construct
EVGSYIVHHSDCDAFYPYCDSSGRSAGGGGTPLGLAGSGGS

SEQ ID NO: 198 - protein, 36 aa, synthetic construct
  REGION 1..36: note = Synthetic Construct
EVGHSDCDAFYPYCDSSGRSAGGGGTPLGLAGSGGS

SEQ ID NO: 199 - protein, 32 aa, synthetic construct
  REGION 1..32: note = Synthetic Construct
EDCDAFYPYCDSSGRSAGGGGTPLGLAGSGGS

SEQ ID NO: 200 - protein, 41 aa, synthetic construct
  REGION 1..41: note = Synthetic Construct
EVGSYPNPSSDCVPYYYACAYSGRSAGGGGTPLGLAGSGGS

SEQ ID NO: 201 - protein, 36 aa, synthetic construct
  REGION 1..36: note = Synthetic Construct
EVGSSDCVPYYYACAYSGRSAGGGGTPLGLAGSGGS

SEQ ID NO: 202 - protein, 32 aa, synthetic construct
  REGION 1..32: note = Synthetic Construct
EDCVPYYYACAYSGRSAGGGGTPLGLAGSGGS

SEQ ID NO: 203 - protein, 118 aa, synthetic construct
  REGION 1..118: note = Synthetic Construct
MHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGAQIYVIDPE

SEQ ID NO: 204 - protein, 118 aa, synthetic construct
  REGION 1..118: note = Synthetic Construct
MHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPE

SEQ ID NO: 205 - protein, 115 aa, synthetic construct
  REGION 1..115: note = Synthetic Construct
KAMHVAQPAVVLASSRGIASFVCEYASPGKATEVRVTVLRQADSQVTEVCAATYMMGNELTFICTGTSSGNQVNLTIQGLRAMDTGLYICKVELMYPPPYYLGIGNGTQIYVIDP

SEQ ID NO: 206 - protein, 114 aa, synthetic construct
  REGION 1..114: note = Synthetic Construct
AMHVAQPAVVLASSRGIASFVCEYASPGKATEVRVTVLRQADSQVTEVCAATYMMGNELTFDSICTGTSSGNQVNLTIQGLRAMDTGLYICKVELMYPPPYYLGIGNGTQIYVI

SEQ ID NO: 207 - protein, 120 aa, Homo sapiens
KAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPE

SEQ ID NO: 208 - protein, 120 aa, Mus musculus
EAIQVTQPSVVLASSHGVASFPCEYSPSHNTDEVRVTVLRQTNDQMTEVCATTFTEKNTVGFLDYPFCSGTFNESRVNLTIQGLRAVDTGLYLCKVELMYPPPYFVGMGNGTQIYVIDPE

SEQ ID NO: 209 - protein, 31 aa, synthetic construct
  REGION 1..31: note = Synthetic Construct
EVGSYPNPSSDCVPYYYACAYSGRSAPLGLA

SEQ ID NO: 210 - protein, 22 aa, synthetic construct
  REGION 1..22: note = Synthetic Construct
EDCVPYYYACAYSGRSAPLGLA

SEQ ID NO: 211 - protein, 17 aa, synthetic construct
  REGION 1..17: note = Synthetic Construct
EDCVPYYYACAYSGRSA

SEQ ID NO: 212 - protein, 17 aa, synthetic construct
  REGION 1..17: note = Synthetic Construct
EDCVPYYYACAYPLGLA

SEQ ID NO: 213 - protein, 12 aa, synthetic construct
  REGION 1..12: note = Synthetic Construct
EDCVPYYYACAY

SEQ ID NO: 214 - protein, 16 aa, synthetic construct
  REGION 1..16: note = Synthetic Construct
EVGSSDCVPYYYACAY

SEQ ID NO: 215 - protein, 12 aa, synthetic construct
  REGION 1..12: note = Synthetic Construct
EDCDAFYPYCDS

SEQ ID NO: 216 - protein, 16 aa, synthetic construct
  REGION 1..16: note = Synthetic Construct
EVGHSDCDAFYPYCDS

SEQ ID NO: 217 - protein, 20 aa, synthetic construct
  REGION 1..20: note = Synthetic Construct
  VARIANT 3: note = Present in repeats of at least one and up to 11
  VARIANT 5..12: note = Xaa = Ala, Cys, Asp, Glu, Phe, Gly, His, Ile, Lys, Leu, Met, Asn, Pro, Gln, Arg, Ser, Thr, Val, Trp, or Tyr
  VARIANT 3: note = Xaa = Asp, Ala, Tyr, Ser, Thr, Asn, Ile, Leu, Phe, Val, His, or Pro
  VARIANT 14..15: note = Xaa = Asp, Ala, Tyr, Ser, Thr, Asn, Ile, Leu, Phe, Val, His, or Pro
EVXCXXXXXXXXCXXSGRSA

SEQ ID NO: 218 - protein, 17 aa, synthetic construct
  REGION 1..17: note = Synthetic Construct
  VARIANT 4..9: note = Xaa = Asp, Ala, Tyr, Ser, Thr, Asn, Ile, Leu, Phe, Val, His, or Pro
  VARIANT 11..12: note = Xaa = Asp, Ala, Tyr, Ser, Thr, Asn, Ile, Leu, Phe, Val, His, or Pro
EDCXXXXXXCXXSGRSA

SEQ ID NO: 219 - protein, 17 aa, synthetic construct
  REGION 1..17: note = Synthetic Construct
  VARIANT 4..9: note = Xaa = Asp, Ala, Tyr, Ser, Thr, Asn, Ile, Leu, Phe, Val, His, or Pro
  VARIANT 11..12: note = Xaa = Asp, Ala, Tyr, Ser, Thr, Asn, Ile, Leu, Phe, Val, His, or Pro
EDCXXXXXXCXXPLGLA

SEQ ID NO: 220 - protein, 21 aa, synthetic construct
  REGION 1..21: note = Synthetic Construct
SGRSAGGGGTENLYFQGSGGS

SEQ ID NO: 221 - protein, 20 aa, synthetic construct
  REGION 1..20: note = Synthetic Construct
SGRSAGGGGTPLGLAGSGGS

SEQ ID NO: 222 - protein, 10 aa, synthetic construct
  REGION 1..10: note = Synthetic Construct
SGRSAPLGLA

SEQ ID NO: 223 - protein, 13 aa, synthetic construct
  REGION 1..13: note = Synthetic Construct
FSLSTGGVGVGWI

SEQ ID NO: 224 - protein, 20 aa, synthetic construct
  REGION 1..20: note = Synthetic Construct
LALIDWADDKYYSPSLKSRL

SEQ ID NO: 225 - protein, 15 aa, synthetic construct
  REGION 1..15: note = Synthetic Construct
ARGGSDTVIGDWFAY

SEQ ID NO: 226 - protein, 11 aa, synthetic construct
  REGION 1..11: note = Synthetic Construct
RASQSIGSYLA

SEQ ID NO: 227 - protein, 9 aa, synthetic construct
  REGION 1..9: note = Synthetic Construct
DASNLETGV

SEQ ID NO: 228 - protein, 10 aa, synthetic construct
  REGION 1..10: note = Synthetic Construct
YCQQGYYLWT

SEQ ID NO: 229 - protein, 123 aa, synthetic construct
  REGION 1..123: note = Synthetic Construct
EVQLVESGGGLVQPGGSLRLSCAASGFSLSTGGVGVGWIRQAPGKGLEWLALIDWADDKYYSPSLKSRLTISRDNSKNTLYLQLNSLRAEDTAVYYCARGGSDTVIGDWFAYWGQGTLVTVSS

SEQ ID NO: 230 - protein, 106 aa, synthetic construct
  REGION 1..106: note = Synthetic Construct
DIQLTQSPSSLSASVGDRVTITCRASQSIGSYLAWYQQKPGKAPKLLIYDASNLETGVPSRFSGSGSGTDFTLTISSLQPEDFATYYCQQGYYLWTFGQGTKVEIK

SEQ ID NO: 231-311 - skipped (81 entries)

SEQ ID NO: 312 - protein, 5 aa, synthetic construct
DYEMH

SEQ ID NO: 313 - protein, 17 aa, synthetic construct
VIESETGGTAYNQKFKG

SEQ ID NO: 314 - protein, 16 aa, synthetic construct
EGITTVATTYYWYFDV

SEQ ID NO: 315 - protein, 16 aa, synthetic construct
RSSQSIVHSNGNTYLE

SEQ ID NO: 316 - protein, 7 aa, synthetic construct
KVSNRFS

SEQ ID NO: 317 - protein, 9 aa, synthetic construct
FQGSHVPLT

SEQ ID NO: 318 - protein, 125 aa, synthetic construct [sequence omitted]

SEQ ID NO: 319 - protein, 112 aa, synthetic construct [sequence omitted]

SEQ ID NO: 320 - protein, 445 aa, synthetic construct
EVQLVESGGGLVQPGGSLRLSCAASGYSISSGYHWSWIRQAPGKGLEWLARIDWDDDKYYSTSLKSRLTISRDNSKNTLYLQLNSLRAEDTAVYYCARSYVYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 321 - protein, 446 aa, synthetic construct
EVQLVESGGGLVQPGGSLRLSCAASGYSISSGYHWSWIRQAPGKGLEWLARIDWDDDKYYSTSLKSRLTISRDNSKNTLYLQLNSLRAEDTAVYYCARSYVYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 322 - protein, 256 aa, synthetic construct
EVGSYPNPSSDCVPYYYACAYSGRSAGGGGTPLGLAGSGGSDIQLTQSPSSLSASVGDRVTITCRASQSVRGRFLAWYQQKPGKAPKLLIYDASNRATGIPSRFSGSGSGTDFTLTISSLQPEDFATYYCQQSSSWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 323 - protein, 445 aa, synthetic construct; identical to SEQ ID NO: 320

SEQ ID NO: 324 - protein, 446 aa, synthetic construct; identical to SEQ ID NO: 321

SEQ ID NO: 325 - protein, 256 aa, synthetic construct
EVGSYNFVADSCPDHPYPCSASGRSAGGGGSPLGLAGSGGSDIQLTQSPSSLSASVGDRVTITCRASQSVRGRFLAWYQQKPGKAPKLLIYDASNRATGIPSRFSGSGSGTDFTLTISSLQPEDFATYYCQQSSSWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 326 - protein, 445 aa, synthetic construct; identical to SEQ ID NO: 320

SEQ ID NO: 327 - protein, 446 aa, synthetic construct; identical to SEQ ID NO: 321

SEQ ID NO: 328 - protein, 256 aa, synthetic construct
EVGSYIVHHSDCDAFYPYCDSSGRSAGGGGSPLGLAGSGGSDIQLTQSPSSLSASVGDRVTITCRASQSVRGRFLAWYQQKPGKAPKLLIYDASNRATGIPSRFSGSGSGTDFTLTISSLQPEDFATYYCQQSSSWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC